(19) United States
(12) Patent Application Publication
(10) Pub. No.: US 2024/0217916 A1
(43) Pub. Date: Jul. 4, 2024

(54) ORGANIC COMPOUND AND USE THEREOF

(71) Applicants: Wuhan Tianma Microelectronics Co., Ltd., Wuhan (CN); Wuhan Tianma Microelectronics Co., Ltd. Shanghai Branch, Shanghai (CN)

(72) Inventor: Wenpeng DAI, Wuhan (CN)

(73) Assignees: Wuhan Tianma Microelectronics Co., Ltd., Wuhan (CN); Wuhan Tianma Microelectronics Co., Ltd. Shanghai Branch, Shanghai (CN)

(21) Appl. No.: 18/588,068

(22) Filed: Feb. 27, 2024

(30) Foreign Application Priority Data
Sep. 26, 2023 (CN) 202311255169.7

(51) Int. Cl. C07C 211/61; C07D 215/38; C07D 221/20; C07D 239/74; C07D 241/44; C07D 307/91; C07D 471/04; C07F 7/08; H10K 85/60

(52) U.S. Cl. CPC C07C 211/61; C07D 215/38; C07D 221/20; C07D 239/74; C07D 241/44; C07D 307/91; C07D 471/04; C07F 7/0816; H10K 85/633; H10K 85/636; H10K 85/656; H10K 85/6572; H10K 85/6574

(57) ABSTRACT

Provided are an organic compound and use thereof, where the organic compound has a structure represented by Formula I. The use of organic compound as a material of the capping layer in combination with a capping layer material containing high-refractive-index small organic molecules is conducive to improving the efficiency of a device, in particular improving the color cast.

This application claims priority to Chinese Patent Application No. 202311255169.7, filed on Sep. 26, 2023, the contents of which are incorporated herein in its entirety by reference.

### TECHNICAL FIELD

The present disclosure belongs to the field of organic electroluminescent materials and relates to an organic compound and use thereof.

### BACKGROUND

After decades of development, organic light-emitting diodes (OLEDs) have gained considerable progress. Although the internal quantum efficiency of the OLED reaches approximately 100%, the external quantum efficiency is only about 20%. Most light is confined inside a light-emitting device due to factors such as a substrate mode loss, a surface plasmon loss and a waveguide effect, resulting in the loss of a large amount of energy.

In a top-emitting device, an organic capping layer (CPL) is deposited through evaporation on a translucent metal electrode Al so that an optical interference distance is adjusted, the reflection of external light is suppressed, and the extinction caused by the movement of surface plasmon is suppressed, thereby improving light extraction efficiency and light-emitting efficiency.

By designing the bi-layer CPL device, the photochromic efficiency of the device can be improved, and the power consumption can be effectively reduced. However, the refractive index of the existing organic capping layer material is generally about 1.8, and the molecular volume and polarity of most mass-produced materials are such that the molecular refractive index is mostly concentrated in the range of 1.75-1.95, thereby failing to meet the requirements of the high refractive index. Therefore, it is important in the art for further development of organic capping layer materials.

### SUMMARY

In view of the defects in the related art, the object of the present disclosure is to provide an organic compound and the use thereof.

To achieve the preceding object, the present disclosure adopts the following technical solutions.

A first object of the present disclosure is to provide an organic compound. The organic compound has a structure represented by Formula I:

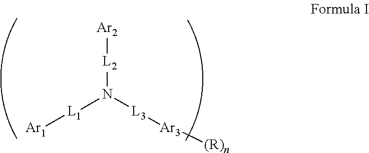

where L₁ to L₃ are independently selected from a single bond, C6-C18 arylene or C5-C18 heteroarylene;
Ar₁ to Ar₃ are each independently selected from one of substituted or unsubstituted C₆-C30 aryl or substituted or unsubstituted C3-C30 heteroaryl, and at least one of ArL₁ or ArL₁ is selected from any one of the following groups

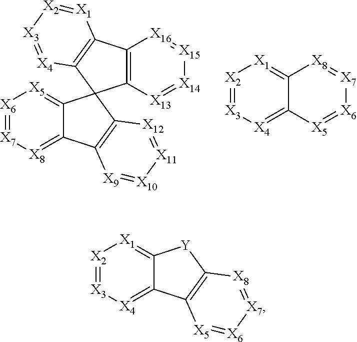

where the site of attachment of the group is any attachable position;
X₁ to X₁₆ are independently C—R₁ or N, and R₁ is hydrogen, C6-C18 aryl or C5-C18 heteroaryl; Y is O, S, N or Si;
R is F, —CHF₂, CH₂F, —CF₃ or —CN, where n is the number of R and is an integer greater than or equal to 1.

In the present disclosure, the use of the organic compound having the structure described above in organic electroluminescent devices enables the devices to have a low drive voltage, a high current efficiency and a long lifetime and can improve the color cast.

In the present disclosure, C6-C18 may each independently be C7, C8, C9, C10, C12, C13, C14, C15, C16, or C18.

C5-C18 may each independently be C6, C8, C9, C10, C12, C13, C14, C15, C16, or C18.

C6-C30 may each independently be C7, C8, C9, C10, C12, C13, C14, C15, C16, C18, C20, C22, C24, C26, C28, C29, etc.

C3-C30 may each independently be C3, C5, C6, C7, C8, C9, C10, C12, C13, C14, C15, C16, C18, C20, C22, C24, C26, C28, C29, etc.

A second object of the present disclosure is to provide an organic

electroluminescent material including the organic compound as described in the first object.

A third object of the present disclosure is to provide an organic capping layer material including the organic compound as described in the first object.

A fourth object of the present disclosure is to provide an OLED device. The OLED device includes an anode, a cathode, and an organic thin-film layer disposed between the anode and the cathode, where the material of the organic thin-film layer includes the organic compound as described in the first object.

A fifth object of the present disclosure is to provide an OLED device. The OLED includes an anode, a cathode, an organic thin-film layer disposed between the anode and the cathode, and an organic capping layer disposed above the cathode, where the material of the organic capping layer includes the organic compound as described in the first object.

A sixth object of the present disclosure is to provide a display panel including the OLED device as described in the fourth or fifth object.

A seventh object of the present disclosure is to provide an organic light-emitting display device including the display panel as described in the sixth object.

An eighth object of the present disclosure is to provide an electronic device including the display panel as described in the sixth object.

Compared with the related art, the present disclosure has the beneficial effects described below.

The use of the organic compound provided in the present disclosure as an organic capping layer material enables the organic electroluminescent devices to have a low drive voltage, a high current efficiency and a long lifetime and can improve the color cast.

### DETAILED DESCRIPTION

Technical solutions of the present disclosure are further described below through embodiments. It is to be understood by those skilled in the art that the embodiments described below are used for a better understanding of the present disclosure and are not to be construed as specific limitations to the present disclosure.

A first object of the present disclosure is to provide an organic compound. The organic compound has a structure represented by Formula I:

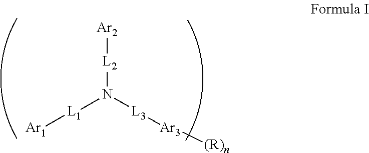

where L₁ to L₃ are independently selected from a single bond, C6-C18 arylene or C5-C18 heteroarylene;
Ar₁ to Ar₃ are each independently selected from one of substituted or unsubstituted C6-C30 aryl or substituted or unsubstituted C3-C30 heteroaryl, and at least one of Ar₁ or Ar₃ is selected from any one of the following groups

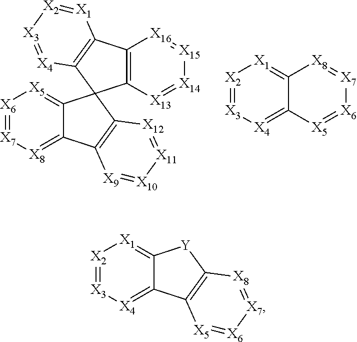

where the site of attachment of the group is any attachable position;
X₁ to X₁₆ are independently C—R₁ or N, and R₁ is hydrogen, C6-C18 aryl or C5-C18 heteroaryl; Y is O, S, N or Si;
R is F, —CHF₂, CH₂F, —CF₃ or —CN, where n is the number of R and is an integer greater than or equal to 1 (for example, 1, 2, 3, 4, 5, 6, 7, 8, 9, 10, etc.).

In the present disclosure, C6-C18 may each independently be C7, C8, C9, C10, C12, C13, C14, C15, C16, or C18.

C5-C18 may each independently be C6, C8, C9, C10, C12, C13, C14, C15, C16, or C18.

C6-C30 may each independently be C7, C8, C9, C10, C12, C13, C14, C15, C16, C18, C20, C22, C24, C26, C28, C29, etc.

C3-C30 may each independently be C3, C5, C6, C7, C8, C9, C10, C12, C13, C14, C15, C16, C18, C20, C22, C24, C26, C28, C29, etc.

In one embodiment, L₁ to L₃ are independently selected from any one of a single bond, substituted or unsubstituted phenyl, substituted or unsubstituted biphenyl, substituted or unsubstituted naphthyl, substituted or unsubstituted phenanthryl, substituted or unsubstituted triphenylene, substituted or unsubstituted fluorenyl, substituted or unsubstituted carbazolyl, substituted or unsubstituted dibenzofuryl, substituted or unsubstituted dibenzothienyl, substituted or unsubstituted pyridyl, substituted or unsubstituted pyrimidinyl, or substituted or unsubstituted triazinyl; the substituent in the substituted group is selected from fluorine, trifluoromethyl, cyano, methyl, ethyl, t-butyl, isopropyl or methoxy.

In one embodiment, Ar₁ to Ar₃ are independently selected from any one of phenyl, biphenyl, naphthyl,

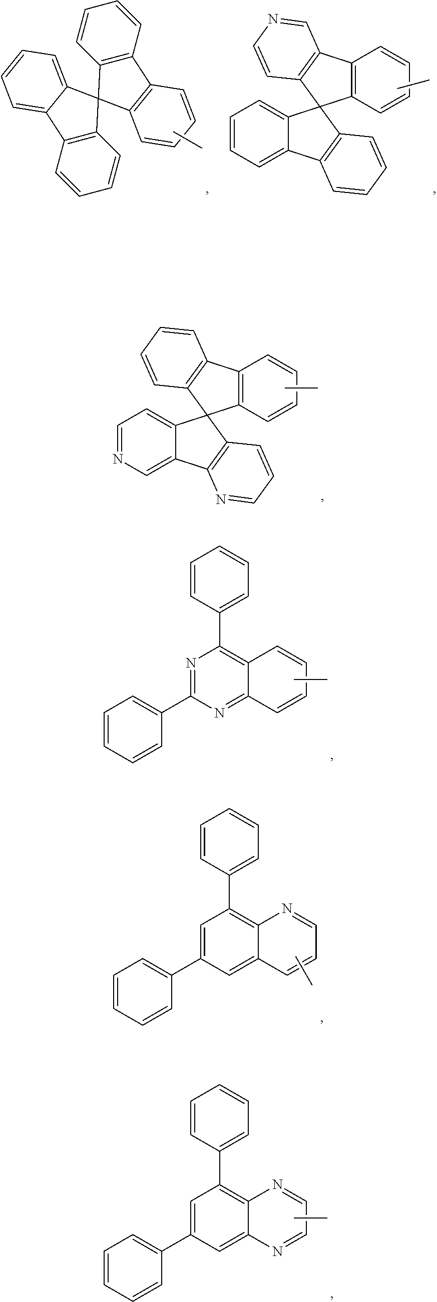

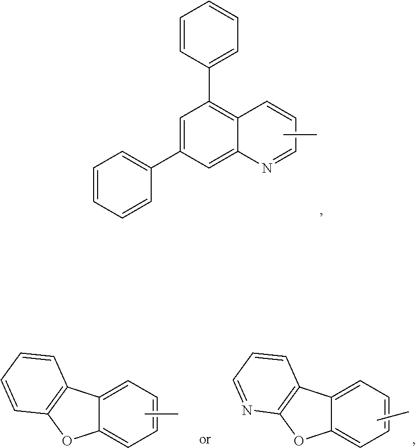

and at least one of Ar₁ to Ar₃ is selected from any one of naphthyl,

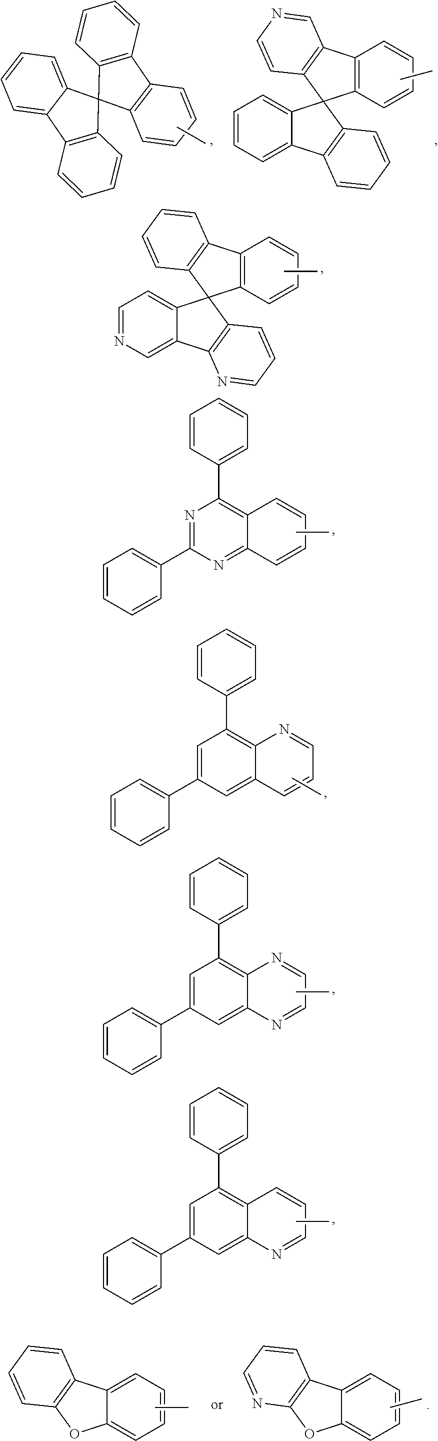

In one embodiment, n is an integer of 1 to 3, for example, 1, 2 or 3.

In one embodiment, the organic compound is any one of the following compounds:

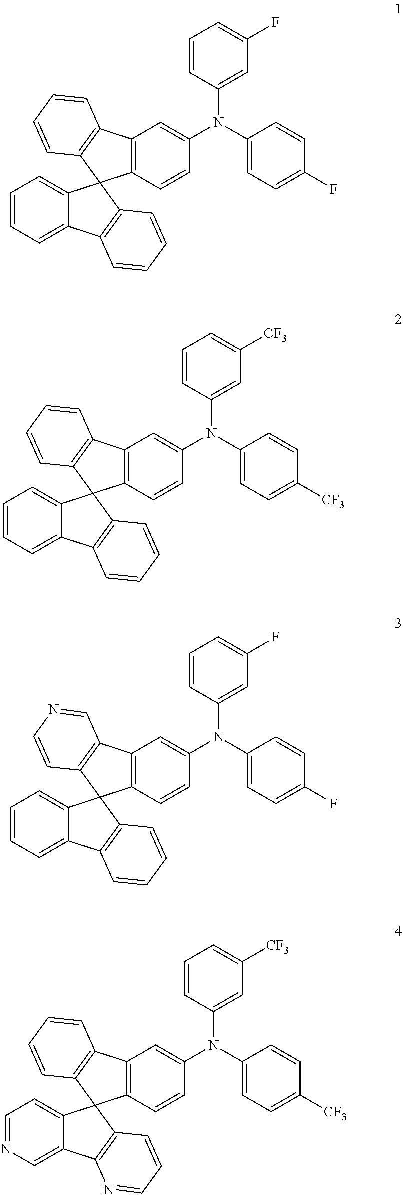 [SMILES: Fc1ccc(N(c2cccc(F)c2)c2ccc3c(c2)-c2ccccc2C32c3ccccc3-c3ccccc32)cc1 ; FC(F)(F)c1ccc(N(c2cccc(C(F)(F)F)c2)c2ccc3c(c2)-c2ccccc2C32c3ccccc3-c3ccccc32)cc1 ; Fc1ccc(N(c2cccc(F)c2)c2ccc3c(c2)-c2cnccc2C32c3ccccc3-c3ccccc32)cc1 ; FC(F)(F)c1ccc(N(c2cccc(C(F)(F)F)c2)c2ccc3c(c2)-c2ccccc2C32c3ccncc3-c3ncccc32)cc1]

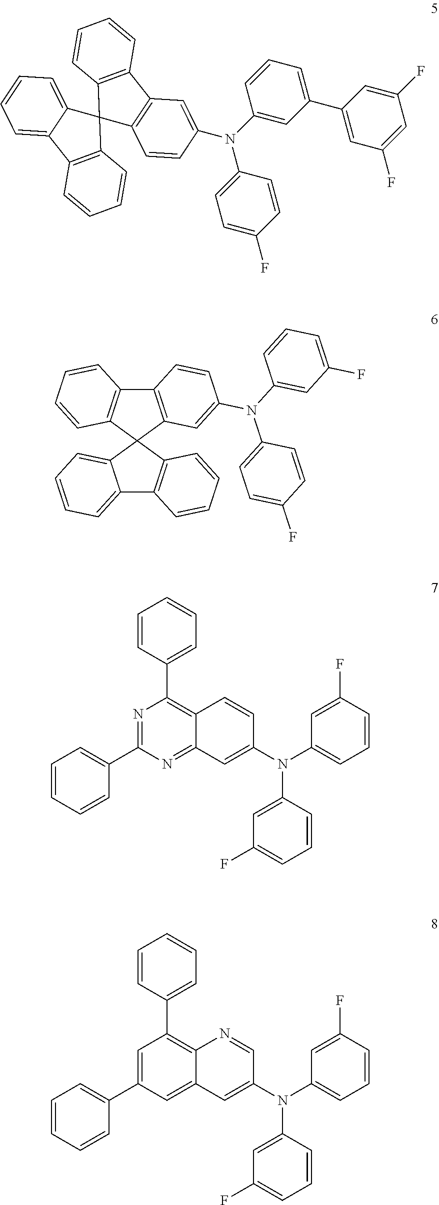 [SMILES: Fc1ccc(N(c2cccc(-c3cc(F)cc(F)c3)c2)c2ccc3c(c2)-c2ccccc2C32c3ccccc3-c3ccccc32)cc1 ; Fc1ccc(N(c2cccc(F)c2)c2ccc3c(c2)C2(c4ccccc4-c4ccccc42)c2ccccc2-3)cc1 ; Fc1cccc(N(c2cccc(F)c2)c2ccc3c(-c4ccccc4)nc(-c4ccccc4)nc3c2)c1 ; Fc1cccc(N(c2cccc(F)c2)c2cnc3c(-c4ccccc4)cc(-c4ccccc4)cc3c2)c1]

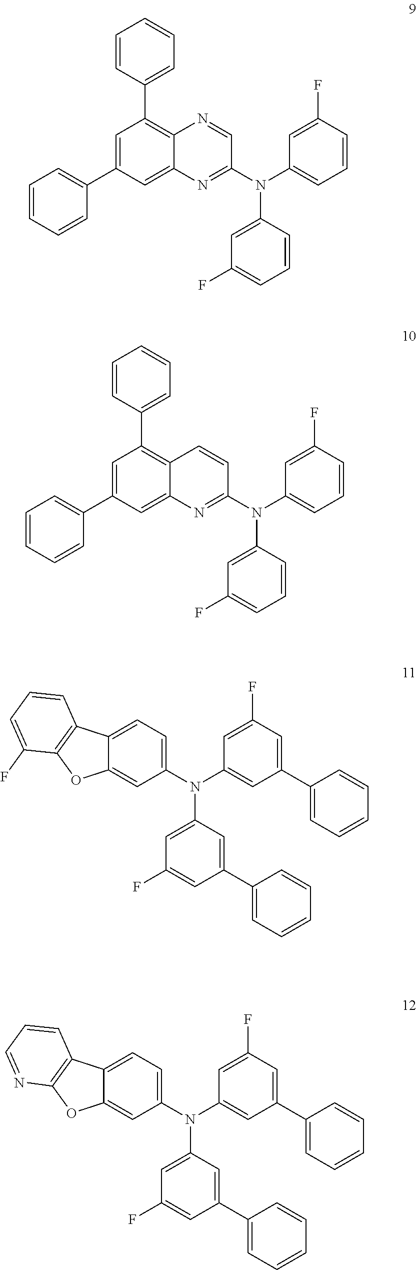 [SMILES: Fc1cccc(N(c2cccc(F)c2)c2cnc3c(-c4ccccc4)cc(-c4ccccc4)cc3n2)c1 ; Fc1cccc(N(c2cccc(F)c2)c2ccc3c(-c4ccccc4)cc(-c4ccccc4)cc3n2)c1 ; Fc1cc(-c2ccccc2)cc(N(c2cc(F)cc(-c3ccccc3)c2)c2ccc3c(c2)oc2c(F)cccc23)c1 ; Fc1cc(-c2ccccc2)cc(N(c2cc(F)cc(-c3ccccc3)c2)c2ccc3c(c2)oc2ncccc23)c1]

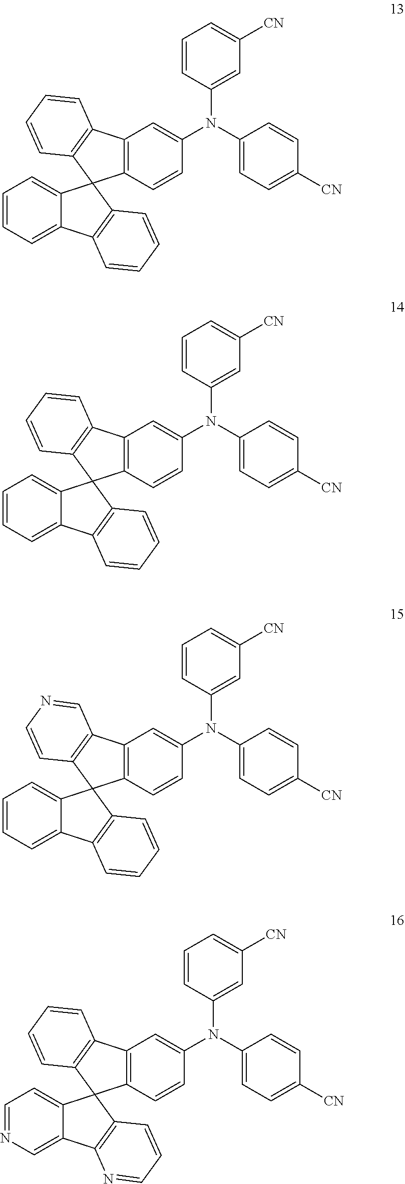 [SMILES: N#Cc1ccc(N(c2cccc(C#N)c2)c2ccc3c(c2)-c2ccccc2C32c3ccccc3-c3ccccc32)cc1 ; N#Cc1ccc(N(c2cccc(C#N)c2)c2ccc3c(c2)-c2ccccc2C32c3ccccc3-c3ccccc32)cc1 ; N#Cc1ccc(N(c2cccc(C#N)c2)c2ccc3c(c2)-c2cnccc2C32c3ccccc3-c3ccccc32)cc1 ; N#Cc1ccc(N(c2cccc(C#N)c2)c2ccc3c(c2)-c2ccccc2C32c3ccncc3-c3ncccc32)cc1]

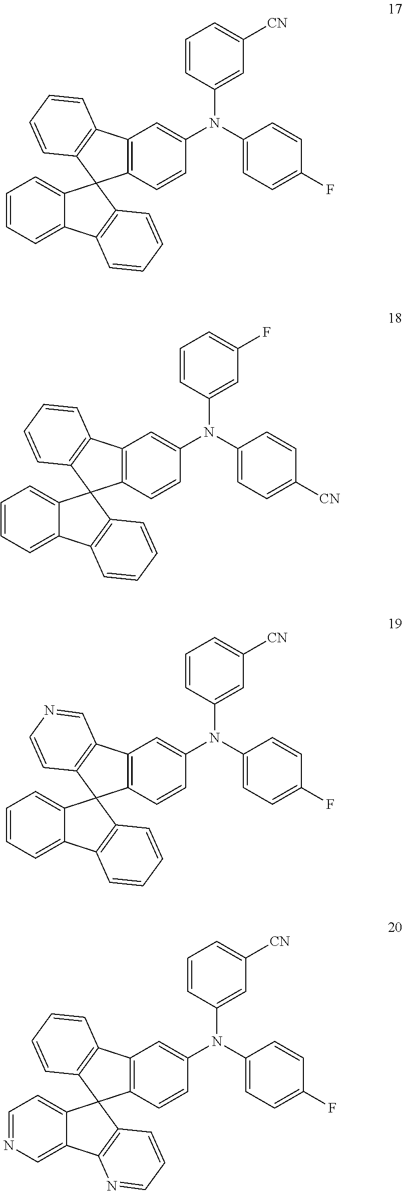 [SMILES: N#Cc1cccc(N(c2ccc(F)cc2)c2ccc3c(c2)-c2ccccc2C32c3ccccc3-c3ccccc32)c1 ; N#Cc1ccc(N(c2cccc(F)c2)c2ccc3c(c2)-c2ccccc2C32c3ccccc3-c3ccccc32)cc1 ; N#Cc1cccc(N(c2ccc(F)cc2)c2ccc3c(c2)-c2cnccc2C32c3ccccc3-c3ccccc32)c1 ; N#Cc1cccc(N(c2ccc(F)cc2)c2ccc3c(c2)-c2ccccc2C32c3ccncc3-c3ncccc32)c1]

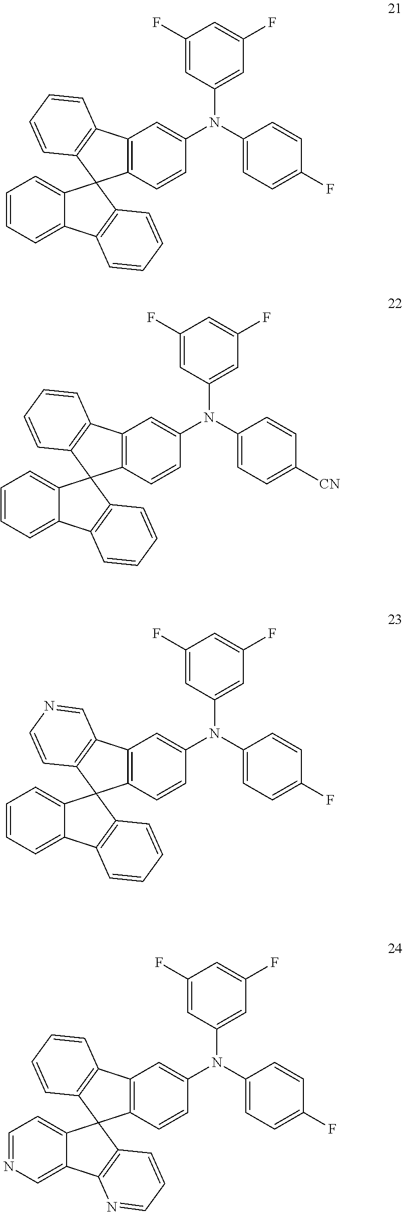 [SMILES: Fc1ccc(N(c2cc(F)cc(F)c2)c2ccc3c(c2)-c2ccccc2C32c3ccccc3-c3ccccc32)cc1 ; N#Cc1ccc(N(c2cc(F)cc(F)c2)c2ccc3c(c2)-c2ccccc2C32c3ccccc3-c3ccccc32)cc1 ; Fc1ccc(N(c2cc(F)cc(F)c2)c2ccc3c(c2)-c2cnccc2C32c3ccccc3-c3ccccc32)cc1 ; Fc1ccc(N(c2cc(F)cc(F)c2)c2ccc3c(c2)-c2ccccc2C32c3ccncc3-c3ncccc32)cc1]

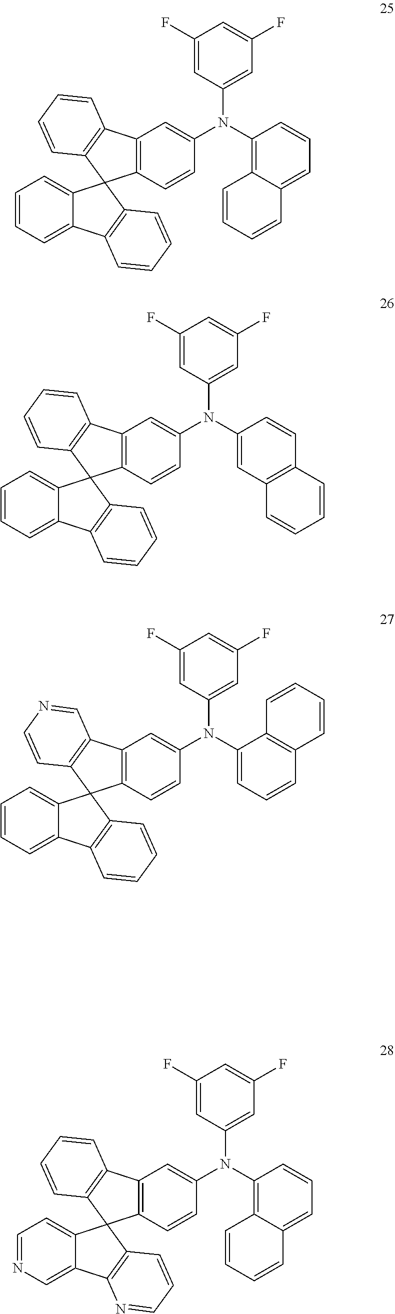 [SMILES: Fc1cc(F)cc(N(c2ccc3c(c2)-c2ccccc2C32c3ccccc3-c3ccccc32)c2cccc3ccccc23)c1 ; Fc1cc(F)cc(N(c2ccc3c(c2)-c2ccccc2C32c3ccccc3-c3ccccc32)c2ccc3ccccc3c2)c1 ; Fc1cc(F)cc(N(c2ccc3c(c2)-c2cnccc2C32c3ccccc3-c3ccccc32)c2cccc3ccccc23)c1 ; Fc1cc(F)cc(N(c2ccc3c(c2)-c2ccccc2C32c3ccncc3-c3ncccc32)c2cccc3ccccc23)c1]

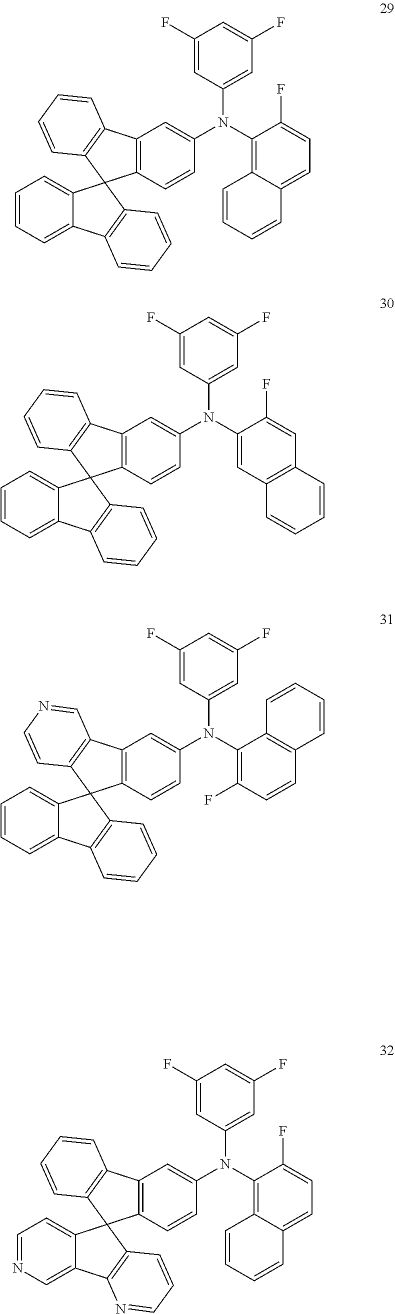 [SMILES: Fc1cc(F)cc(N(c2ccc3c(c2)-c2ccccc2C32c3ccccc3-c3ccccc32)c2c(F)ccc3ccccc23)c1 ; Fc1cc(F)cc(N(c2ccc3c(c2)-c2ccccc2C32c3ccccc3-c3ccccc32)c2cc3ccccc3cc2F)c1 ; Fc1cc(F)cc(N(c2ccc3c(c2)-c2cnccc2C32c3ccccc3-c3ccccc32)c2c(F)ccc3ccccc23)c1 ; Fc1cc(F)cc(N(c2ccc3c(c2)-c2ccccc2C32c3ccncc3-c3ncccc32)c2c(F)ccc3ccccc23)c1]

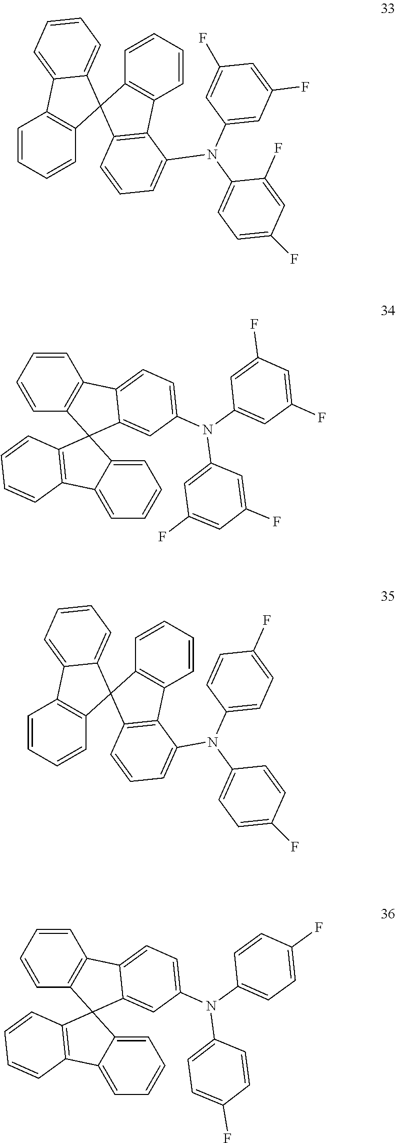 [SMILES: Fc1cc(F)cc(N(c2ccc(F)cc2F)c2cccc3c2-c2ccccc2C32c3ccccc3-c3ccccc32)c1 ; Fc1cc(F)cc(N(c2cc(F)cc(F)c2)c2ccc3c(c2)C2(c4ccccc4-c4ccccc42)c2ccccc2-3)c1 ; Fc1ccc(N(c2ccc(F)cc2)c2cccc3c2-c2ccccc2C32c3ccccc3-c3ccccc32)cc1 ; Fc1ccc(N(c2ccc(F)cc2)c2ccc3c(c2)C2(c4ccccc4-c4ccccc42)c2ccccc2-3)cc1]

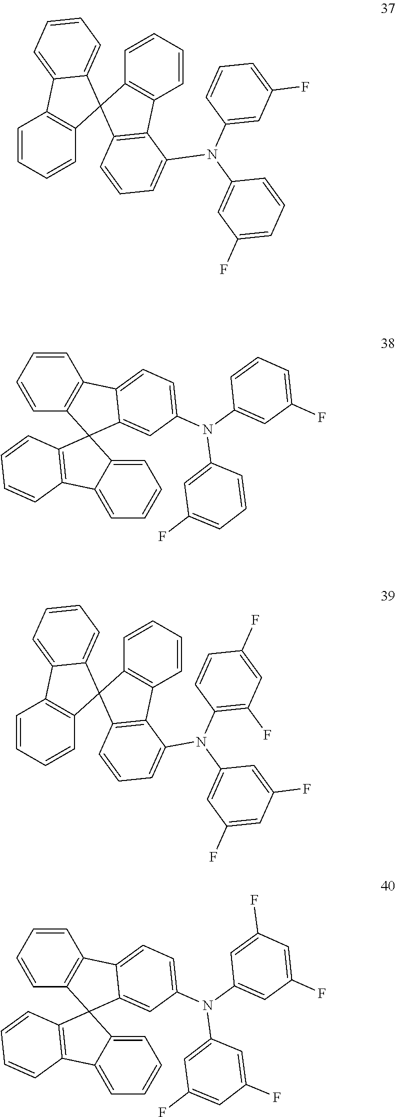 [SMILES: Fc1cccc(N(c2cccc(F)c2)c2cccc3c2-c2ccccc2C32c3ccccc3-c3ccccc32)c1 ; Fc1cccc(N(c2cccc(F)c2)c2ccc3c(c2)C2(c4ccccc4-c4ccccc42)c2ccccc2-3)c1 ; Fc1cc(F)cc(N(c2ccc(F)cc2F)c2cccc3c2-c2ccccc2C32c3ccccc3-c3ccccc32)c1 ; Fc1cc(F)cc(N(c2cc(F)cc(F)c2)c2ccc3c(c2)C2(c4ccccc4-c4ccccc42)c2ccccc2-3)c1]

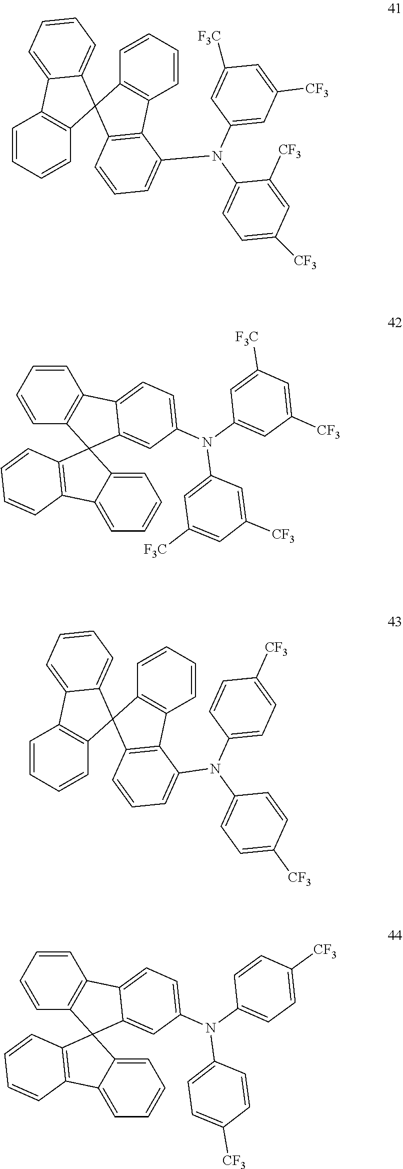 [SMILES: FC(F)(F)c1cc(N(c2ccc(C(F)(F)F)cc2C(F)(F)F)c2cccc3c2-c2ccccc2C32c3ccccc3-c3ccccc32)cc(C(F)(F)F)c1 ; FC(F)(F)c1cc(N(c2cc(C(F)(F)F)cc(C(F)(F)F)c2)c2ccc3c(c2)C2(c4ccccc4-c4ccccc42)c2ccccc2-3)cc(C(F)(F)F)c1 ; FC(F)(F)c1ccc(N(c2ccc(C(F)(F)F)cc2)c2cccc3c2-c2ccccc2C32c3ccccc3-c3ccccc32)cc1 ; FC(F)(F)c1ccc(N(c2ccc(C(F)(F)F)cc2)c2ccc3c(c2)C2(c4ccccc4-c4ccccc42)c2ccccc2-3)cc1]

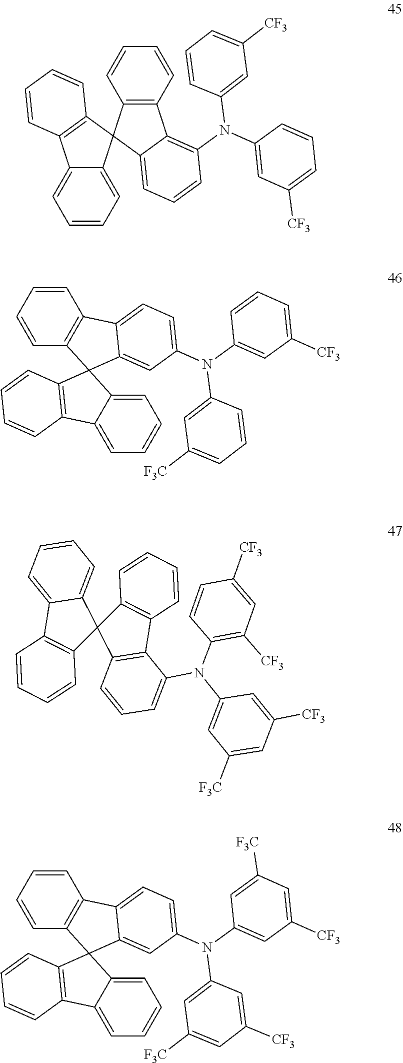 [SMILES: FC(F)(F)c1cccc(N(c2cccc(C(F)(F)F)c2)c2cccc3c2-c2ccccc2C32c3ccccc3-c3ccccc32)c1 ; FC(F)(F)c1cccc(N(c2cccc(C(F)(F)F)c2)c2ccc3c(c2)C2(c4ccccc4-c4ccccc42)c2ccccc2-3)c1 ; FC(F)(F)c1cc(N(c2ccc(C(F)(F)F)cc2C(F)(F)F)c2cccc3c2-c2ccccc2C32c3ccccc3-c3ccccc32)cc(C(F)(F)F)c1 ; FC(F)(F)c1cc(N(c2cc(C(F)(F)F)cc(C(F)(F)F)c2)c2ccc3c(c2)C2(c4ccccc4-c4ccccc42)c2ccccc2-3)cc(C(F)(F)F)c1]

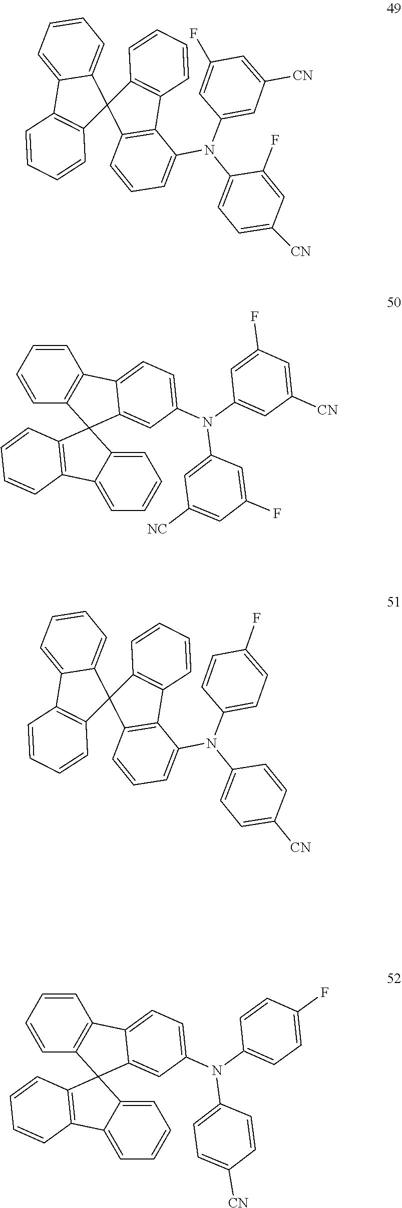 [SMILES: N#Cc1cc(F)cc(N(c2ccc(C#N)cc2F)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1 ; N#Cc1ccc(N(c2ccc(F)cc2)c2cccc3c2-c2ccccc2C32c3ccccc3-c3ccccc32)cc1 ; N#Cc1ccc(N(c2ccc(F)cc2)c2ccc3c(c2)C2(c4ccccc4-c4ccccc42)c2ccccc2-3)cc1]

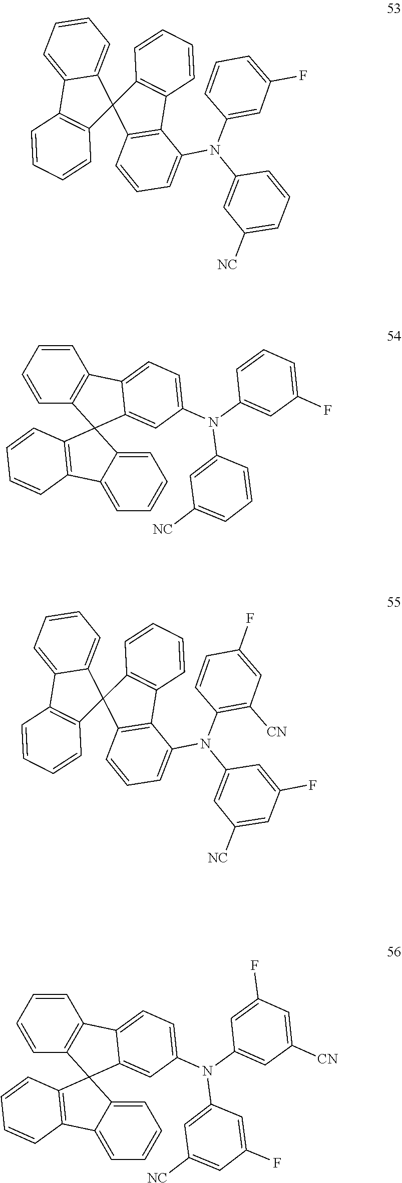 [SMILES: N#Cc1cccc(N(c2cccc(F)c2)c2cccc3c2-c2ccccc2C32c3ccccc3-c3ccccc32)c1 ; N#Cc1cccc(N(c2cccc(F)c2)c2ccc3c(c2)C2(c4ccccc4-c4ccccc42)c2ccccc2-3)c1 ; N#Cc1cc(F)cc(N(c2ccc(F)cc2C#N)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1]

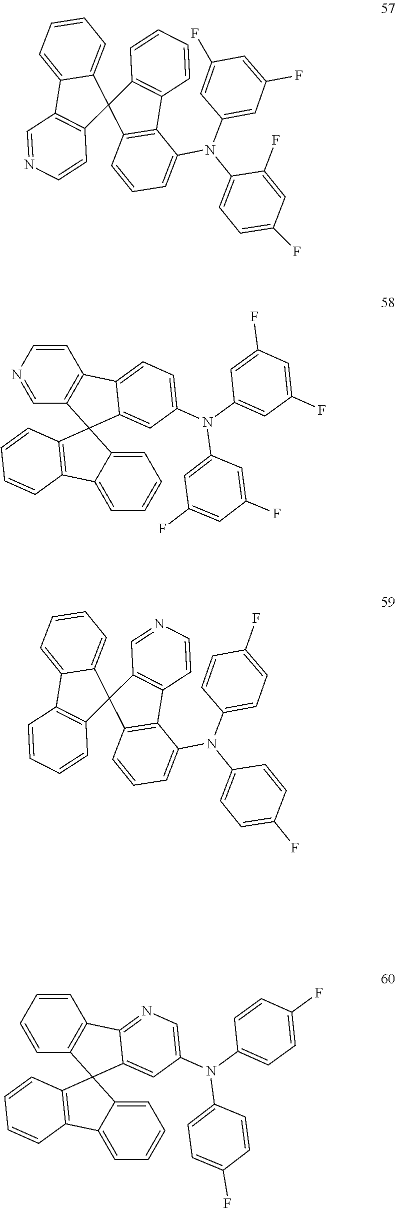 [SMILES: Fc1cc(F)cc(N(c2ccc(F)cc2F)c2cccc3c2-c2ccccc2C32c3ccccc3-c3cnccc32)c1 ; Fc1cc(F)cc(N(c2cc(F)cc(F)c2)c2ccc3c(c2)C2(c4ccccc4-c4ccccc42)c2cnccc2-3)c1 ; Fc1ccc(N(c2ccc(F)cc2)c2cccc3c2-c2ccncc2C32c3ccccc3-c3ccccc32)cc1 ; Fc1ccc(N(c2ccc(F)cc2)c2cnc3c(c2)C2(c4ccccc4-c4ccccc42)c2ccccc2-3)cc1]

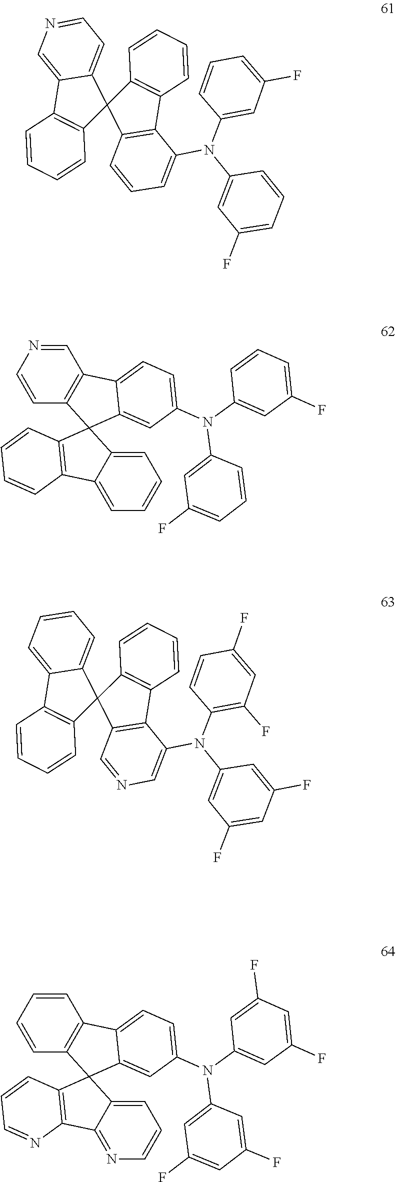 [SMILES: Fc1cccc(N(c2cccc(F)c2)c2cccc3c2-c2ccccc2C32c3ccccc3-c3cnccc32)c1 ; Fc1cccc(N(c2cccc(F)c2)c2ccc3c(c2)C2(c4ccccc4-c4ccccc42)c2ccncc2-3)c1 ; Fc1cc(F)cc(N(c2ccc(F)cc2F)c2cncc3c2-c2ccccc2C32c3ccccc3-c3ccccc32)c1 ; Fc1cc(F)cc(N(c2cc(F)cc(F)c2)c2ccc3c(c2)C2(c4ccccc4-3)c3cccnc3-c3ncccc32)c1]

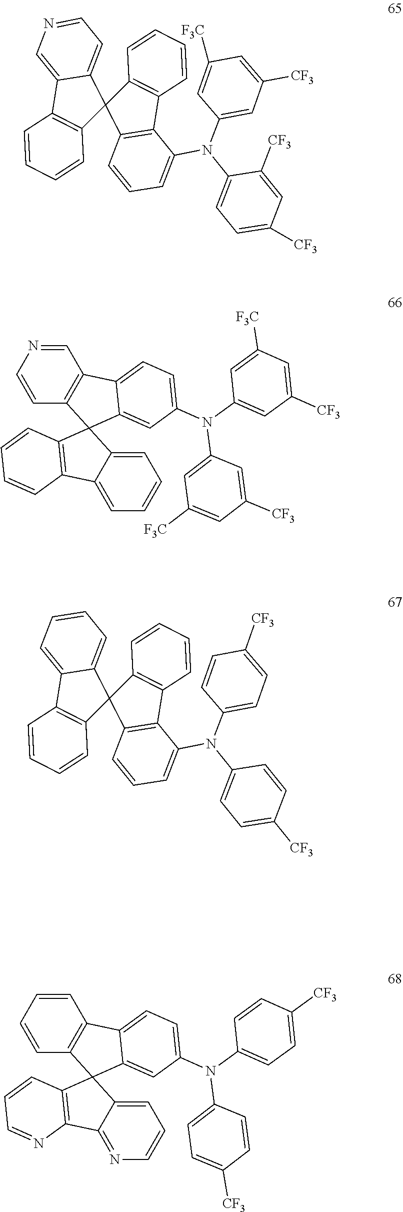 [SMILES: FC(F)(F)c1cc(N(c2ccc(C(F)(F)F)cc2C(F)(F)F)c2cccc3c2-c2ccccc2C32c3ccccc3-c3cnccc32)cc(C(F)(F)F)c1 ; FC(F)(F)c1cc(N(c2cc(C(F)(F)F)cc(C(F)(F)F)c2)c2ccc3c(c2)C2(c4ccccc4-c4ccccc42)c2ccncc2-3)cc(C(F)(F)F)c1 ; FC(F)(F)c1ccc(N(c2ccc(C(F)(F)F)cc2)c2cccc3c2-c2ccccc2C32c3ccccc3-c3ccccc32)cc1 ; FC(F)(F)c1ccc(N(c2ccc(C(F)(F)F)cc2)c2ccc3c(c2)C2(c4ccccc4-3)c3cccnc3-c3ncccc32)cc1]

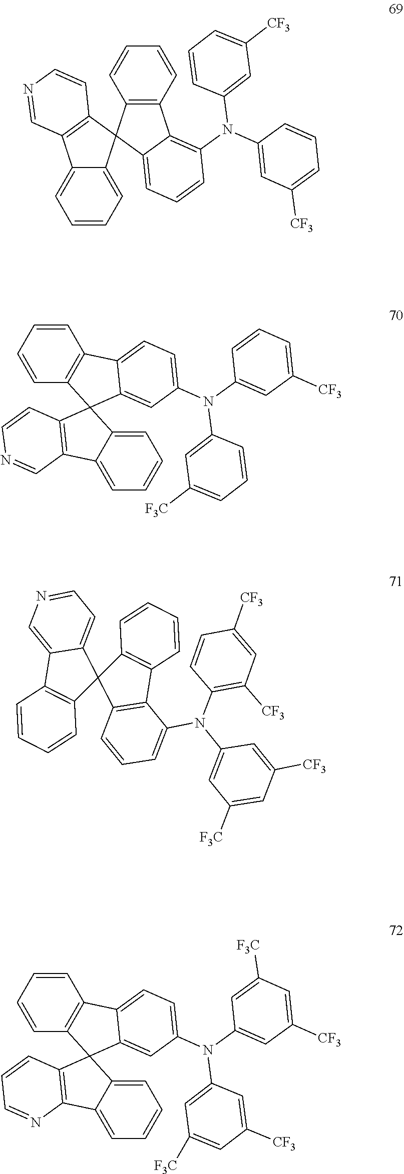 [SMILES: FC(F)(F)c1cccc(N(c2cccc(C(F)(F)F)c2)c2cccc3c2-c2ccccc2C32c3ccccc3-c3cnccc32)c1 ; FC(F)(F)c1cccc(N(c2cccc(C(F)(F)F)c2)c2ccc3c(c2)C2(c4ccccc4-c4cnccc42)c2ccccc2-3)c1 ; FC(F)(F)c1cc(N(c2ccc(C(F)(F)F)cc2C(F)(F)F)c2cccc3c2-c2ccccc2C32c3ccccc3-c3cnccc32)cc(C(F)(F)F)c1 ; FC(F)(F)c1cc(N(c2cc(C(F)(F)F)cc(C(F)(F)F)c2)c2ccc3c(c2)C2(c4ccccc4-3)c3ccccc3-c3ncccc32)cc(C(F)(F)F)c1]

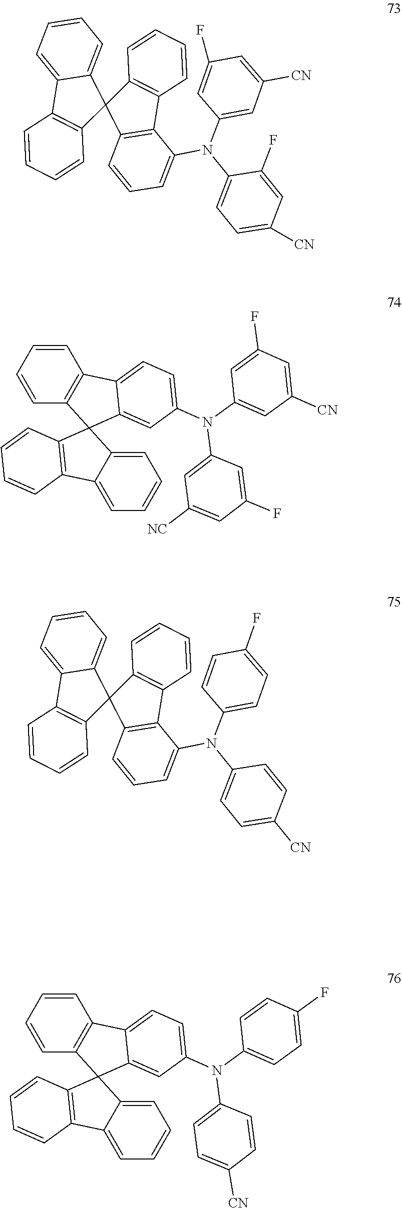 [SMILES: N#Cc1cc(F)cc(N(c2ccc(C#N)cc2F)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1 ; N#Cc1ccc(N(c2ccc(F)cc2)c2cccc3c2-c2ccccc2C32c3ccccc3-c3ccccc32)cc1 ; N#Cc1ccc(N(c2ccc(F)cc2)c2ccc3c(c2)C2(c4ccccc4-c4ccccc42)c2ccccc2-3)cc1]

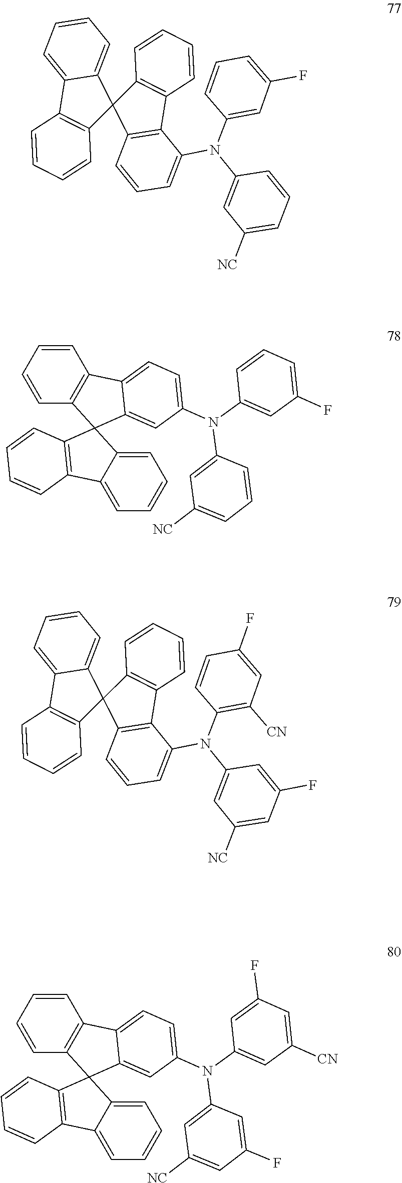 [SMILES: N#Cc1cccc(N(c2cccc(F)c2)c2cccc3c2-c2ccccc2C32c3ccccc3-c3ccccc32)c1 ; N#Cc1cccc(N(c2cccc(F)c2)c2ccc3c(c2)C2(c4ccccc4-c4ccccc42)c2ccccc2-3)c1 ; N#Cc1cc(F)cc(N(c2ccc(F)cc2C#N)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1]

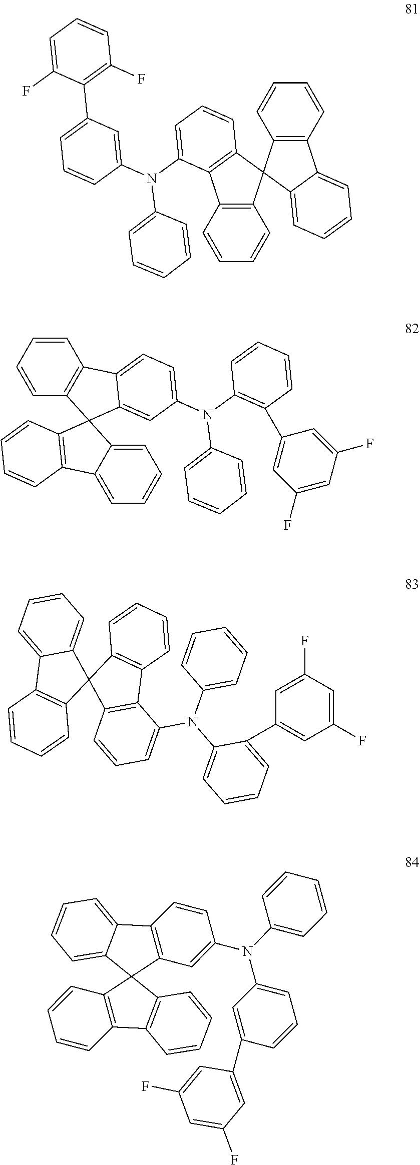 [SMILES: Fc1cccc(F)c1-c1cccc(N(c2ccccc2)c2cccc3c2-c2ccccc2C32c3ccccc3-c3ccccc32)c1 ; Fc1cc(F)cc(-c2ccccc2N(c2ccccc2)c2ccc3c(c2)C2(c4ccccc4-c4ccccc42)c2ccccc2-3)c1 ; Fc1cc(F)cc(-c2ccccc2N(c2ccccc2)c2cccc3c2-c2ccccc2C32c3ccccc3-c3ccccc32)c1 ; Fc1cc(F)cc(-c2cccc(N(c3ccccc3)c3ccc4c(c3)C3(c5ccccc5-c5ccccc53)c3ccccc3-4)c2)c1]

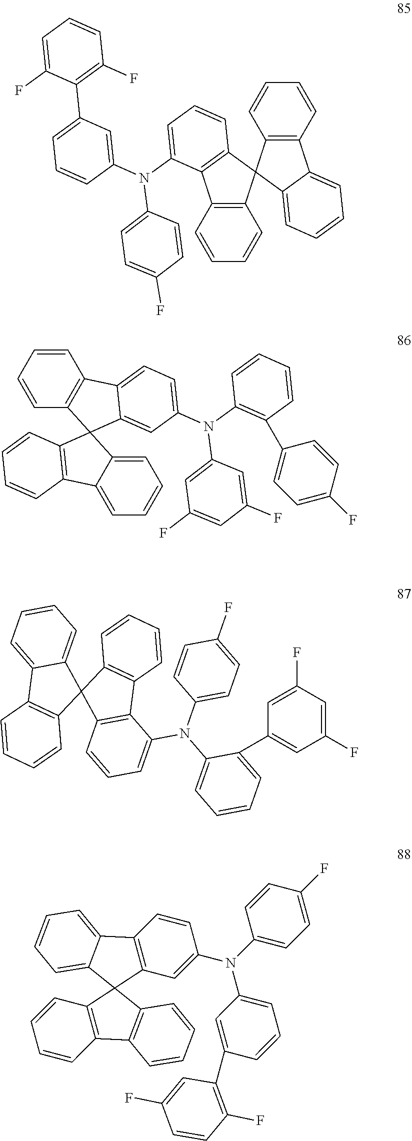 [SMILES: Fc1ccc(N(c2cccc(-c3c(F)cccc3F)c2)c2cccc3c2-c2ccccc2C32c3ccccc3-c3ccccc32)cc1 ; Fc1ccc(-c2ccccc2N(c2cc(F)cc(F)c2)c2ccc3c(c2)C2(c4ccccc4-c4ccccc42)c2ccccc2-3)cc1 ; Fc1ccc(N(c2ccccc2-c2cc(F)cc(F)c2)c2cccc3c2-c2ccccc2C32c3ccccc3-c3ccccc32)cc1 ; Fc1ccc(N(c2cccc(-c3cc(F)ccc3F)c2)c2ccc3c(c2)C2(c4ccccc4-c4ccccc42)c2ccccc2-3)cc1]

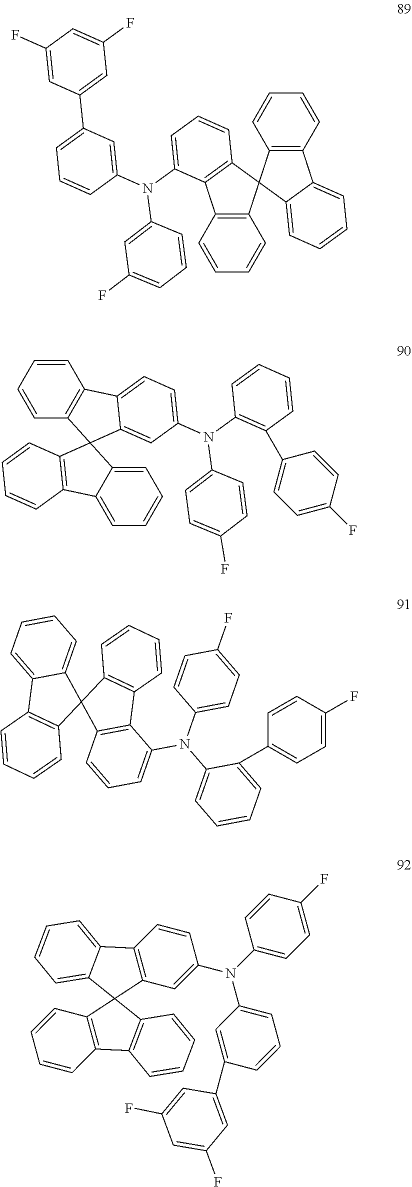 [SMILES: Fc1cc(F)cc(-c2cccc(N(c3cccc(F)c3)c3cccc4c3-c3ccccc3C43c4ccccc4-c4ccccc43)c2)c1 ; Fc1ccc(-c2ccccc2N(c2ccc(F)cc2)c2ccc3c(c2)C2(c4ccccc4-c4ccccc42)c2ccccc2-3)cc1 ; Fc1ccc(-c2ccccc2N(c2ccc(F)cc2)c2cccc3c2-c2ccccc2C32c3ccccc3-c3ccccc32)cc1 ; Fc1ccc(N(c2cccc(-c3cc(F)cc(F)c3)c2)c2ccc3c(c2)C2(c4ccccc4-c4ccccc42)c2ccccc2-3)cc1]

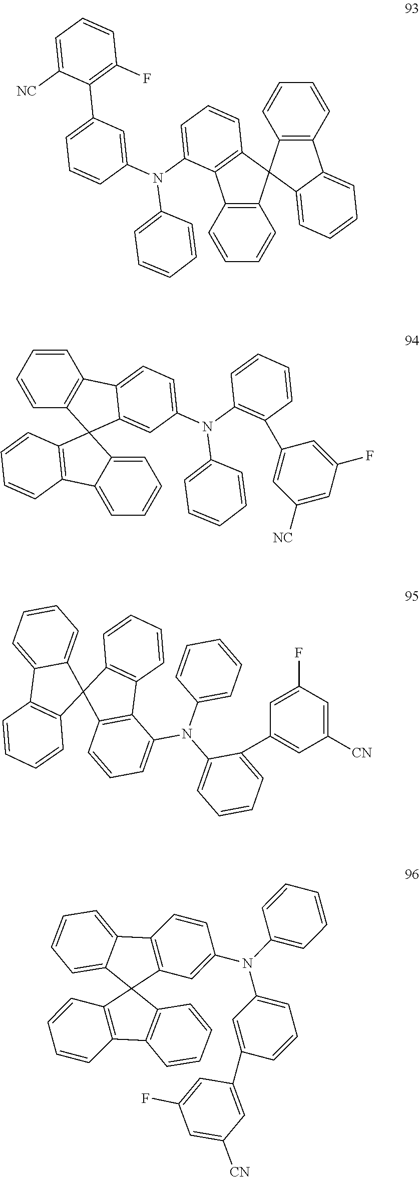 [SMILES: N#Cc1cccc(F)c1-c1cccc(N(c2ccccc2)c2cccc3c2-c2ccccc2C32c3ccccc3-c3ccccc32)c1 ; N#Cc1cc(F)cc(-c2ccccc2N(c2ccccc2)c2ccc3c(c2)C2(c4ccccc4-c4ccccc42)c2ccccc2-3)c1 ; N#Cc1cc(F)cc(-c2ccccc2N(c2ccccc2)c2cccc3c2-c2ccccc2C32c3ccccc3-c3ccccc32)c1 ; N#Cc1cc(F)cc(-c2cccc(N(c3ccccc3)c3ccc4c(c3)C3(c5ccccc5-c5ccccc53)c3ccccc3-4)c2)c1]

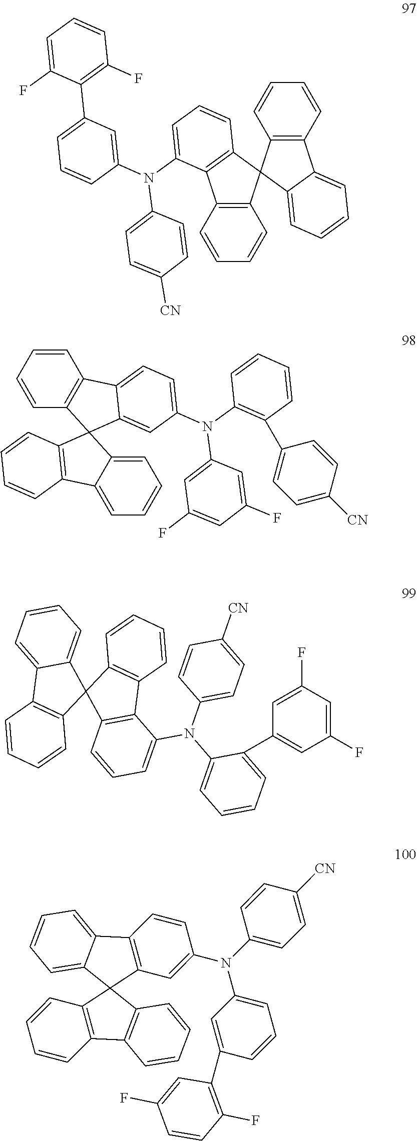 [SMILES: N#Cc1ccc(N(c2cccc(-c3c(F)cccc3F)c2)c2cccc3c2-c2ccccc2C32c3ccccc3-c3ccccc32)cc1 ; N#Cc1ccc(-c2ccccc2N(c2cc(F)cc(F)c2)c2ccc3c(c2)C2(c4ccccc4-c4ccccc42)c2ccccc2-3)cc1 ; N#Cc1ccc(N(c2ccccc2-c2cc(F)cc(F)c2)c2cccc3c2-c2ccccc2C32c3ccccc3-c3ccccc32)cc1 ; N#Cc1ccc(N(c2cccc(-c3cc(F)ccc3F)c2)c2ccc3c(c2)C2(c4ccccc4-c4ccccc42)c2ccccc2-3)cc1]

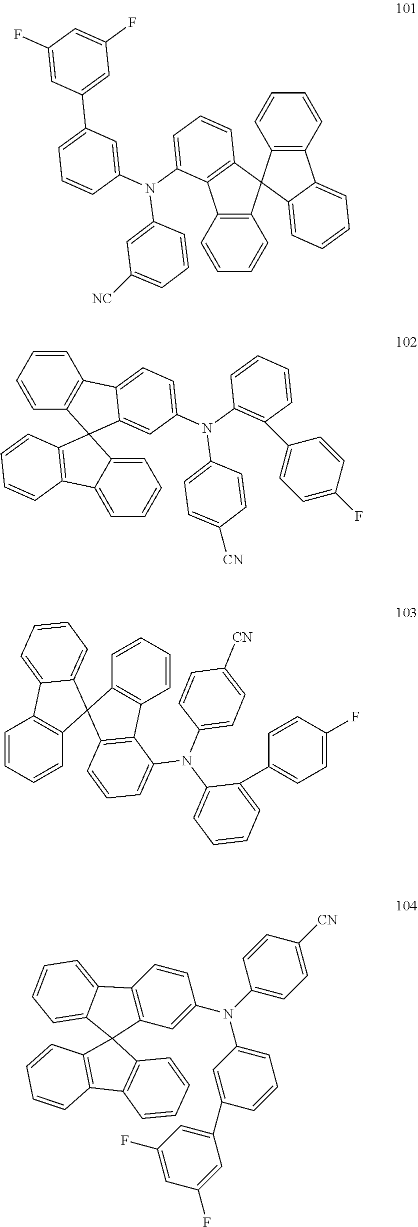 [SMILES: N#Cc1cccc(N(c2cccc(-c3cc(F)cc(F)c3)c2)c2cccc3c2-c2ccccc2C32c3ccccc3-c3ccccc32)c1 ; N#Cc1ccc(N(c2ccc3c(c2)C2(c4ccccc4-c4ccccc42)c2ccccc2-3)c2ccccc2-c2ccc(F)cc2)cc1 ; N#Cc1ccc(N(c2ccccc2-c2ccc(F)cc2)c2cccc3c2-c2ccccc2C32c3ccccc3-c3ccccc32)cc1 ; N#Cc1ccc(N(c2cccc(-c3cc(F)cc(F)c3)c2)c2ccc3c(c2)C2(c4ccccc4-c4ccccc42)c2ccccc2-3)cc1]

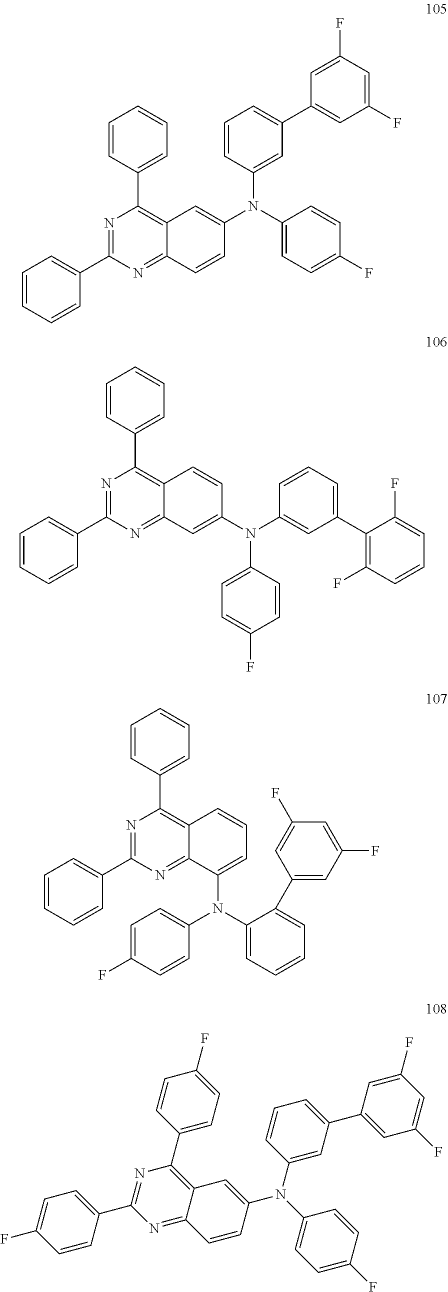 [SMILES: Fc1ccc(N(c2cccc(-c3cc(F)cc(F)c3)c2)c2ccc3nc(-c4ccccc4)nc(-c4ccccc4)c3c2)cc1 ; Fc1ccc(N(c2cccc(-c3c(F)cccc3F)c2)c2ccc3c(-c4ccccc4)nc(-c4ccccc4)nc3c2)cc1 ; Fc1ccc(N(c2ccccc2-c2cc(F)cc(F)c2)c2cccc3c(-c4ccccc4)nc(-c4ccccc4)nc23)cc1 ; Fc1ccc(-c2nc(-c3ccc(F)cc3)c3cc(N(c4ccc(F)cc4)c4cccc(-c5cc(F)cc(F)c5)c4)ccc3n2)cc1]

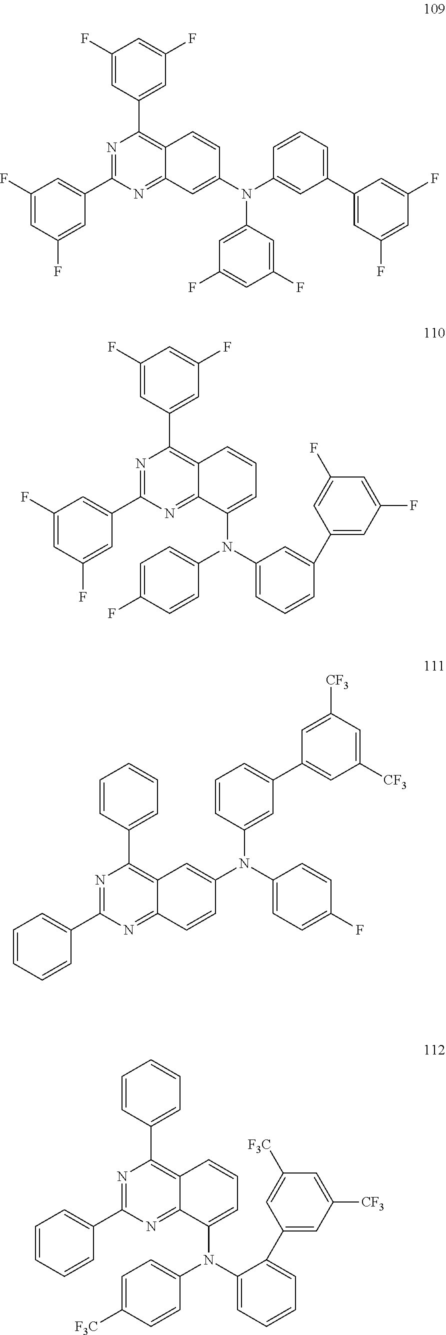 [SMILES: Fc1cc(F)cc(-c2cccc(N(c3cc(F)cc(F)c3)c3ccc4c(-c5cc(F)cc(F)c5)nc(-c5cc(F)cc(F)c5)nc4c3)c2)c1 ; Fc1ccc(N(c2cccc(-c3cc(F)cc(F)c3)c2)c2cccc3c(-c4cc(F)cc(F)c4)nc(-c4cc(F)cc(F)c4)nc23)cc1 ; Fc1ccc(N(c2cccc(-c3cc(C(F)(F)F)cc(C(F)(F)F)c3)c2)c2ccc3nc(-c4ccccc4)nc(-c4ccccc4)c3c2)cc1 ; FC(F)(F)c1ccc(N(c2ccccc2-c2cc(C(F)(F)F)cc(C(F)(F)F)c2)c2cccc3c(-c4ccccc4)nc(-c4ccccc4)nc23)cc1]

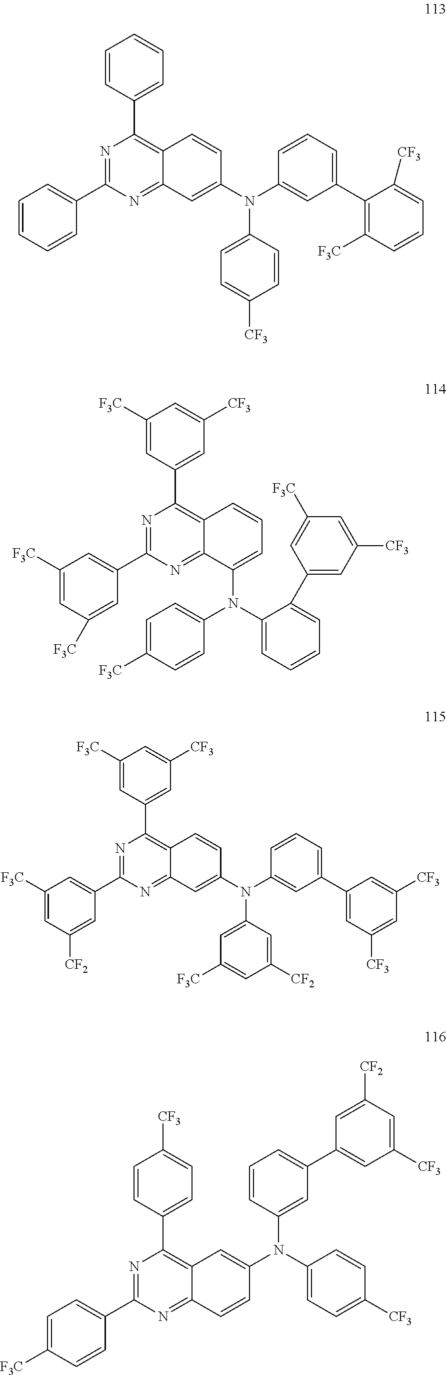

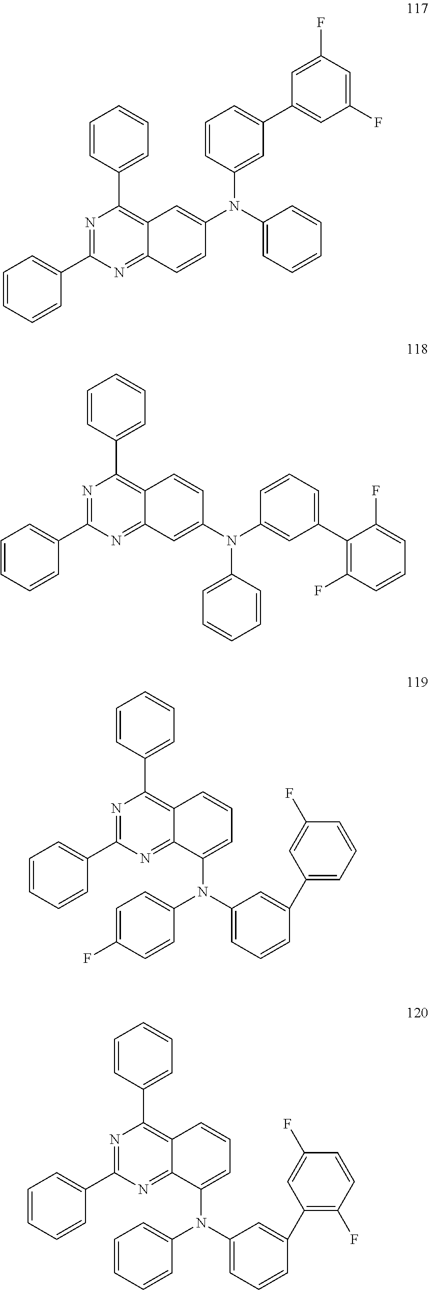 [SMILES: Fc1cc(F)cc(-c2cccc(N(c3ccccc3)c3ccc4nc(-c5ccccc5)nc(-c5ccccc5)c4c3)c2)c1 ; Fc1cccc(F)c1-c1cccc(N(c2ccccc2)c2ccc3c(-c4ccccc4)nc(-c4ccccc4)nc3c2)c1 ; Fc1ccc(N(c2cccc(-c3cccc(F)c3)c2)c2cccc3c(-c4ccccc4)nc(-c4ccccc4)nc23)cc1 ; Fc1ccc(F)c(-c2cccc(N(c3ccccc3)c3cccc4c(-c5ccccc5)nc(-c5ccccc5)nc34)c2)c1]

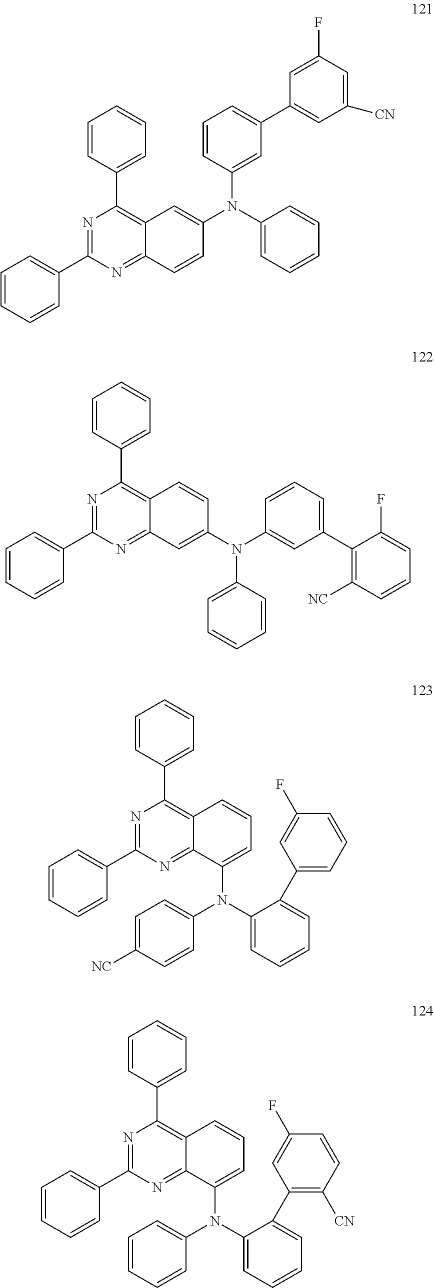 [SMILES: N#Cc1cc(F)cc(-c2cccc(N(c3ccccc3)c3ccc4nc(-c5ccccc5)nc(-c5ccccc5)c4c3)c2)c1 ; N#Cc1cccc(F)c1-c1cccc(N(c2ccccc2)c2ccc3c(-c4ccccc4)nc(-c4ccccc4)nc3c2)c1 ; N#Cc1ccc(N(c2ccccc2-c2cccc(F)c2)c2cccc3c(-c4ccccc4)nc(-c4ccccc4)nc23)cc1 ; N#Cc1ccc(F)cc1-c1ccccc1N(c1ccccc1)c1cccc2c(-c3ccccc3)nc(-c3ccccc3)nc12]

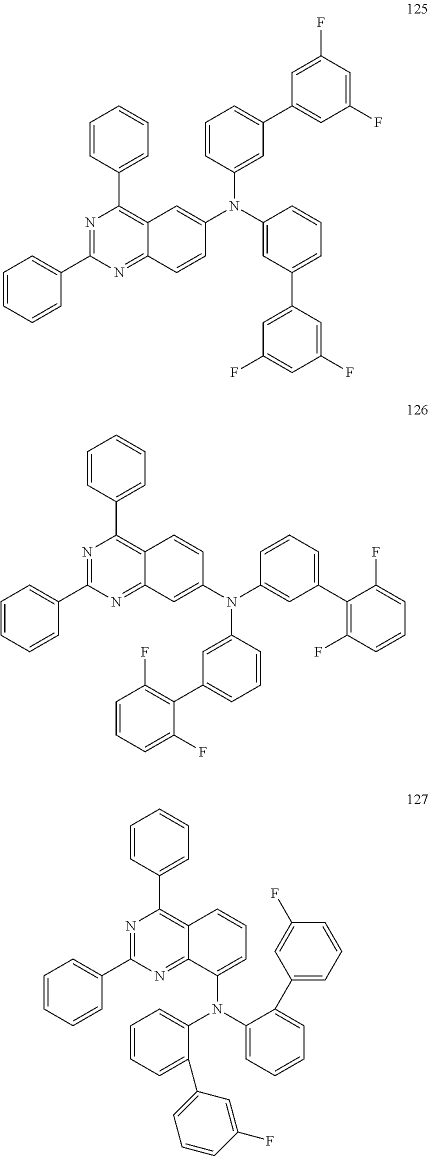 [SMILES: Fc1cc(F)cc(-c2cccc(N(c3cccc(-c4cc(F)cc(F)c4)c3)c3ccc4nc(-c5ccccc5)nc(-c5ccccc5)c4c3)c2)c1 ; Fc1cccc(F)c1-c1cccc(N(c2cccc(-c3c(F)cccc3F)c2)c2ccc3c(-c4ccccc4)nc(-c4ccccc4)nc3c2)c1 ; Fc1cccc(-c2ccccc2N(c2ccccc2-c2cccc(F)c2)c2cccc3c(-c4ccccc4)nc(-c4ccccc4)nc23)c1]

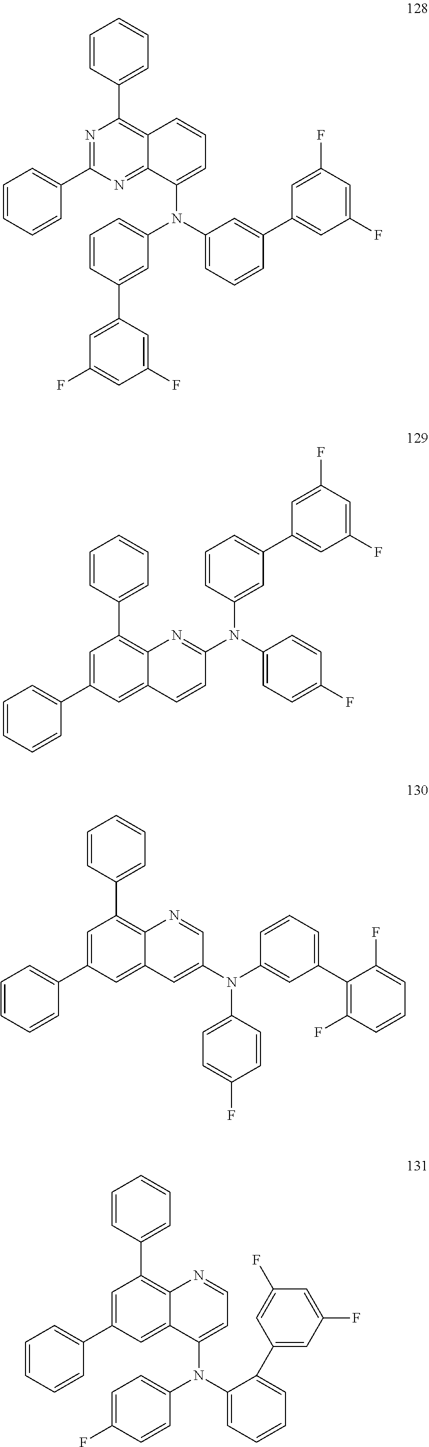 [SMILES: Fc1cc(F)cc(-c2cccc(N(c3cccc(-c4cc(F)cc(F)c4)c3)c3cccc4c(-c5ccccc5)nc(-c5ccccc5)nc34)c2)c1 ; Fc1ccc(N(c2cccc(-c3cc(F)cc(F)c3)c2)c2ccc3cc(-c4ccccc4)cc(-c4ccccc4)c3n2)cc1 ; Fc1ccc(N(c2cccc(-c3c(F)cccc3F)c2)c2cnc3c(-c4ccccc4)cc(-c4ccccc4)cc3c2)cc1 ; Fc1ccc(N(c2ccccc2-c2cc(F)cc(F)c2)c2ccnc3c(-c4ccccc4)cc(-c4ccccc4)cc23)cc1]

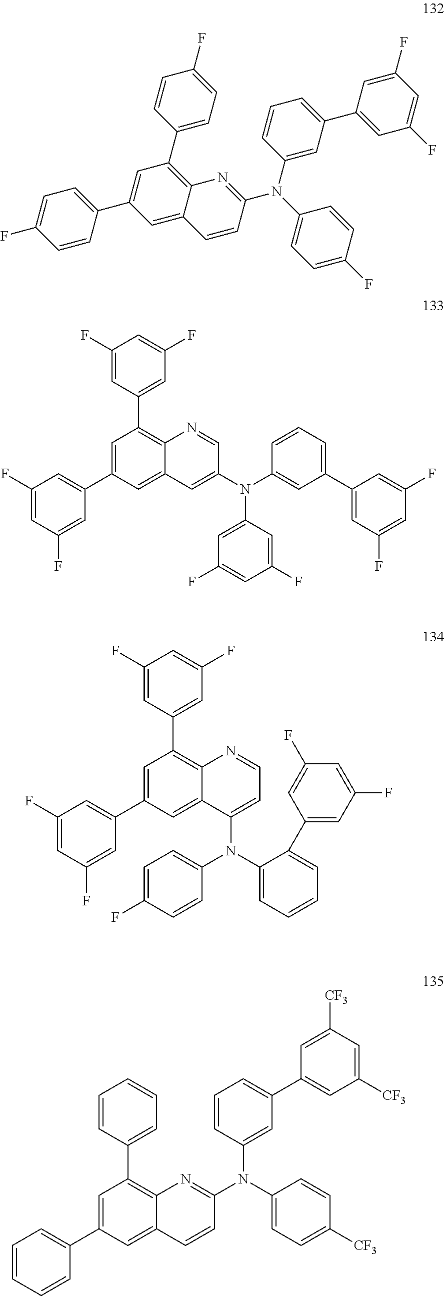 [SMILES: Fc1ccc(-c2cc(-c3ccc(F)cc3)c3nc(N(c4ccc(F)cc4)c4cccc(-c5cc(F)cc(F)c5)c4)ccc3c2)cc1 ; Fc1cc(F)cc(-c2cccc(N(c3cc(F)cc(F)c3)c3cnc4c(-c5cc(F)cc(F)c5)cc(-c5cc(F)cc(F)c5)cc4c3)c2)c1 ; Fc1ccc(N(c2ccccc2-c2cc(F)cc(F)c2)c2ccnc3c(-c4cc(F)cc(F)c4)cc(-c4cc(F)cc(F)c4)cc23)cc1 ; FC(F)(F)c1ccc(N(c2cccc(-c3cc(C(F)(F)F)cc(C(F)(F)F)c3)c2)c2ccc3cc(-c4ccccc4)cc(-c4ccccc4)c3n2)cc1]

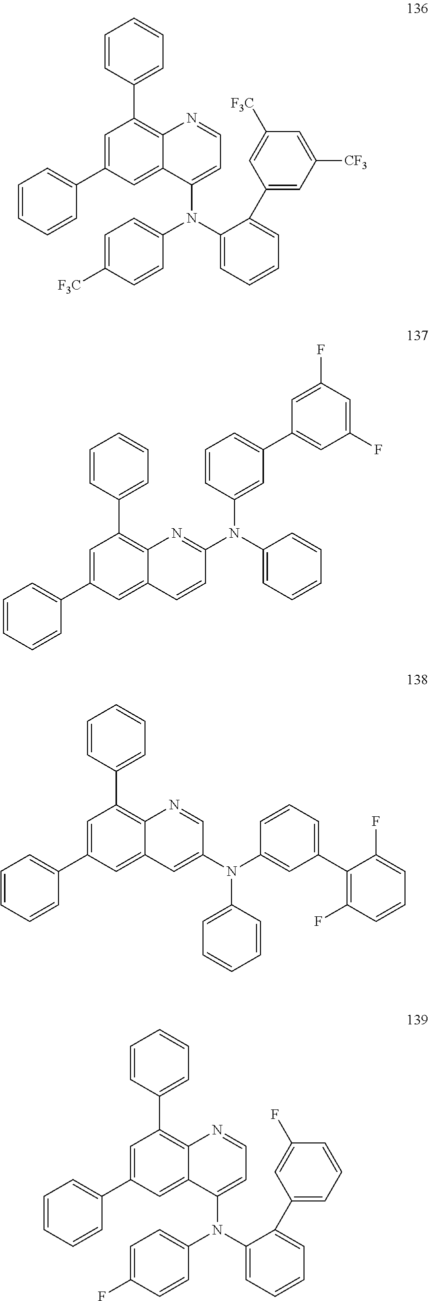 [SMILES: FC(F)(F)c1ccc(N(c2ccccc2-c2cc(C(F)(F)F)cc(C(F)(F)F)c2)c2ccnc3c(-c4ccccc4)cc(-c4ccccc4)cc23)cc1 ; Fc1cc(F)cc(-c2cccc(N(c3ccccc3)c3ccc4cc(-c5ccccc5)cc(-c5ccccc5)c4n3)c2)c1 ; Fc1cccc(F)c1-c1cccc(N(c2ccccc2)c2cnc3c(-c4ccccc4)cc(-c4ccccc4)cc3c2)c1 ; Fc1ccc(N(c2ccccc2-c2cccc(F)c2)c2ccnc3c(-c4ccccc4)cc(-c4ccccc4)cc23)cc1]

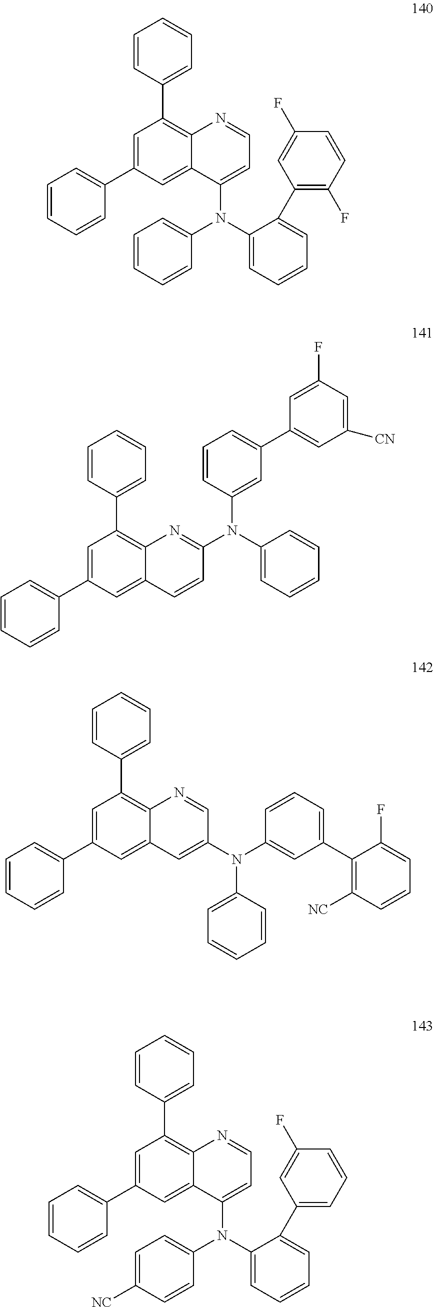 [SMILES: Fc1ccc(F)c(-c2ccccc2N(c2ccccc2)c2ccnc3c(-c4ccccc4)cc(-c4ccccc4)cc23)c1 ; N#Cc1cc(F)cc(-c2cccc(N(c3ccccc3)c3ccc4cc(-c5ccccc5)cc(-c5ccccc5)c4n3)c2)c1 ; N#Cc1cccc(F)c1-c1cccc(N(c2ccccc2)c2cnc3c(-c4ccccc4)cc(-c4ccccc4)cc3c2)c1 ; N#Cc1ccc(N(c2ccccc2-c2cccc(F)c2)c2ccnc3c(-c4ccccc4)cc(-c4ccccc4)cc23)cc1]

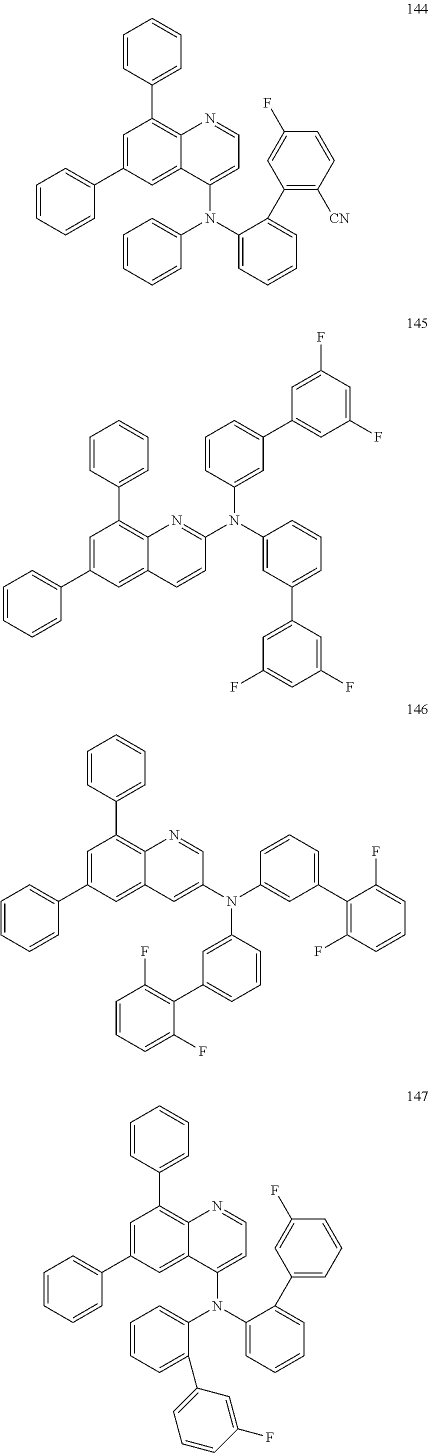 [SMILES: N#Cc1ccc(F)cc1-c1ccccc1N(c1ccccc1)c1ccnc2c(-c3ccccc3)cc(-c3ccccc3)cc12 ; Fc1cc(F)cc(-c2cccc(N(c3cccc(-c4cc(F)cc(F)c4)c3)c3ccc4cc(-c5ccccc5)cc(-c5ccccc5)c4n3)c2)c1 ; Fc1cccc(F)c1-c1cccc(N(c2cccc(-c3c(F)cccc3F)c2)c2cnc3c(-c4ccccc4)cc(-c4ccccc4)cc3c2)c1 ; Fc1cccc(-c2ccccc2N(c2ccccc2-c2cccc(F)c2)c2ccnc3c(-c4ccccc4)cc(-c4ccccc4)cc23)c1]

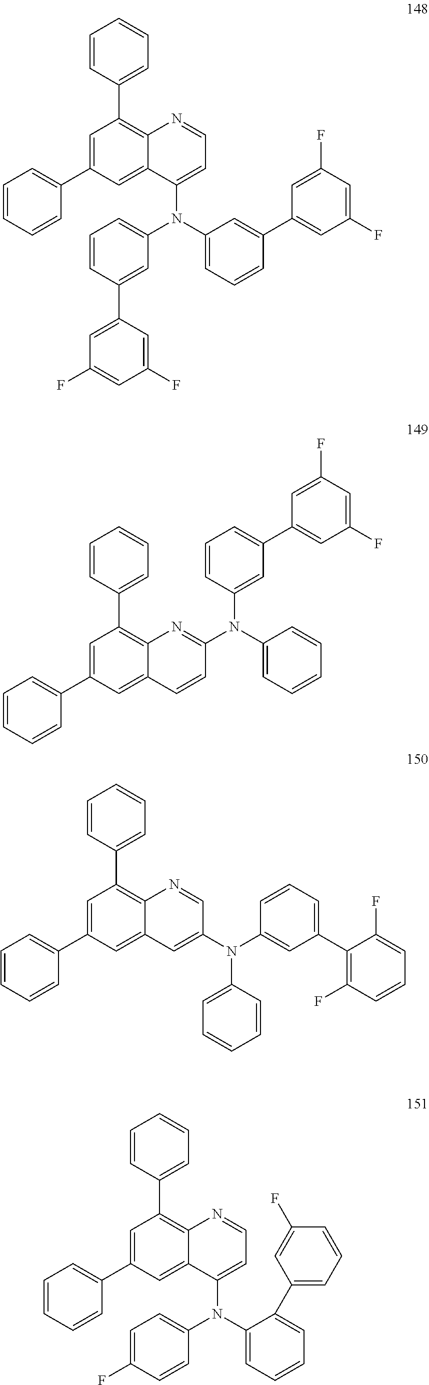 [SMILES: Fc1cc(F)cc(-c2cccc(N(c3cccc(-c4cc(F)cc(F)c4)c3)c3ccnc4c(-c5ccccc5)cc(-c5ccccc5)cc34)c2)c1 ; Fc1cc(F)cc(-c2cccc(N(c3ccccc3)c3ccc4cc(-c5ccccc5)cc(-c5ccccc5)c4n3)c2)c1 ; Fc1cccc(F)c1-c1cccc(N(c2ccccc2)c2cnc3c(-c4ccccc4)cc(-c4ccccc4)cc3c2)c1 ; Fc1ccc(N(c2ccccc2-c2cccc(F)c2)c2ccnc3c(-c4ccccc4)cc(-c4ccccc4)cc23)cc1]

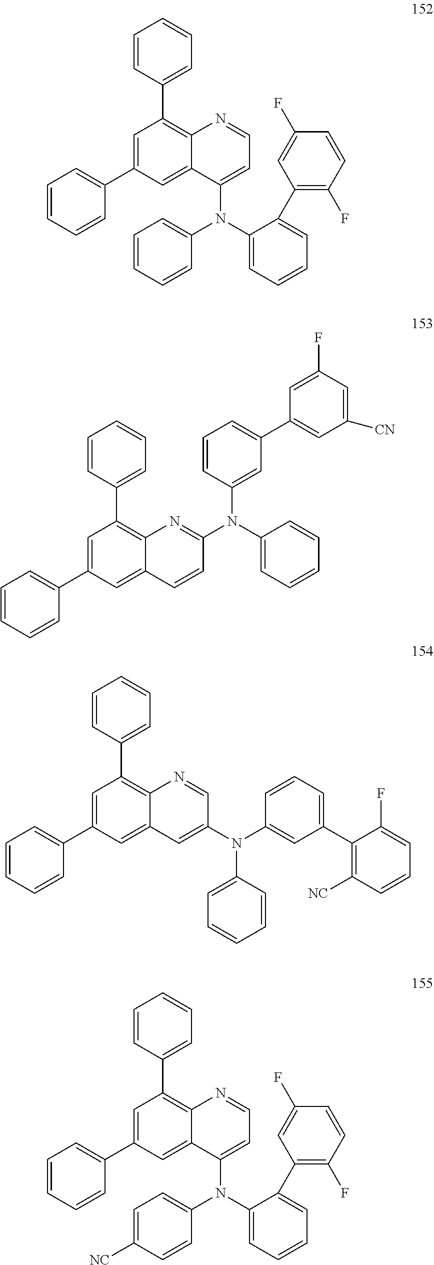 [SMILES: Fc1ccc(F)c(-c2ccccc2N(c2ccccc2)c2ccnc3c(-c4ccccc4)cc(-c4ccccc4)cc23)c1 ; N#Cc1cc(F)cc(-c2cccc(N(c3ccccc3)c3ccc4cc(-c5ccccc5)cc(-c5ccccc5)c4n3)c2)c1 ; N#Cc1cccc(F)c1-c1cccc(N(c2ccccc2)c2cnc3c(-c4ccccc4)cc(-c4ccccc4)cc3c2)c1 ; N#Cc1ccc(N(c2ccccc2-c2cc(F)ccc2F)c2ccnc3c(-c4ccccc4)cc(-c4ccccc4)cc23)cc1]

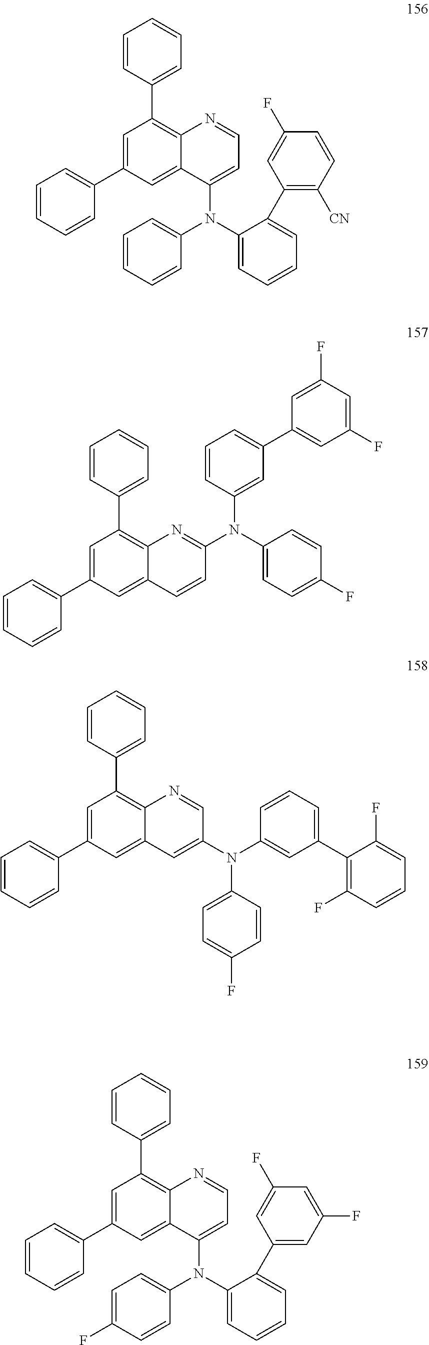 [SMILES: N#Cc1ccc(F)cc1-c1ccccc1N(c1ccccc1)c1ccnc2c(-c3ccccc3)cc(-c3ccccc3)cc12 ; Fc1ccc(N(c2cccc(-c3cc(F)cc(F)c3)c2)c2ccc3cc(-c4ccccc4)cc(-c4ccccc4)c3n2)cc1 ; Fc1ccc(N(c2cccc(-c3c(F)cccc3F)c2)c2cnc3c(-c4ccccc4)cc(-c4ccccc4)cc3c2)cc1 ; Fc1ccc(N(c2ccccc2-c2cc(F)cc(F)c2)c2ccnc3c(-c4ccccc4)cc(-c4ccccc4)cc23)cc1]

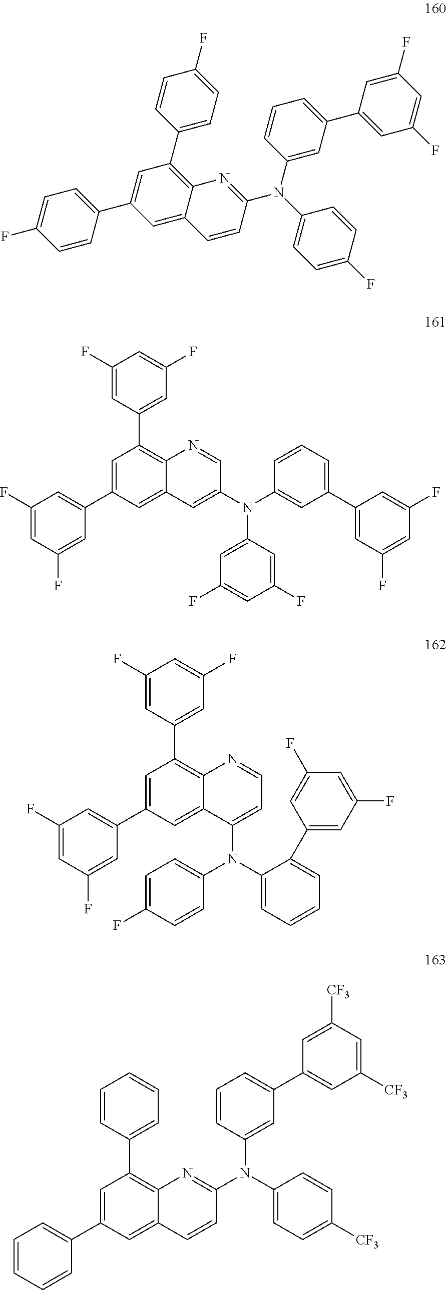 [SMILES: Fc1ccc(-c2cc(-c3ccc(F)cc3)c3nc(N(c4ccc(F)cc4)c4cccc(-c5cc(F)cc(F)c5)c4)ccc3c2)cc1 ; Fc1cc(F)cc(-c2cccc(N(c3cc(F)cc(F)c3)c3cnc4c(-c5cc(F)cc(F)c5)cc(-c5cc(F)cc(F)c5)cc4c3)c2)c1 ; Fc1ccc(N(c2ccccc2-c2cc(F)cc(F)c2)c2ccnc3c(-c4cc(F)cc(F)c4)cc(-c4cc(F)cc(F)c4)cc23)cc1 ; FC(F)(F)c1ccc(N(c2cccc(-c3cc(C(F)(F)F)cc(C(F)(F)F)c3)c2)c2ccc3cc(-c4ccccc4)cc(-c4ccccc4)c3n2)cc1]

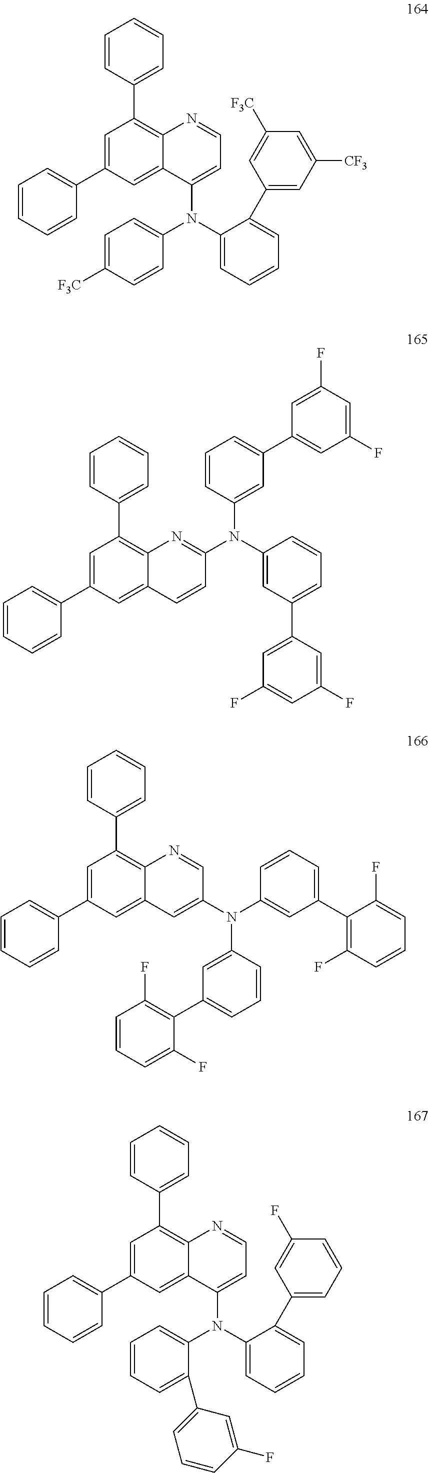 [SMILES: FC(F)(F)c1ccc(N(c2ccccc2-c2cc(C(F)(F)F)cc(C(F)(F)F)c2)c2ccnc3c(-c4ccccc4)cc(-c4ccccc4)cc23)cc1 ; Fc1cc(F)cc(-c2cccc(N(c3cccc(-c4cc(F)cc(F)c4)c3)c3ccc4cc(-c5ccccc5)cc(-c5ccccc5)c4n3)c2)c1 ; Fc1cccc(F)c1-c1cccc(N(c2cccc(-c3c(F)cccc3F)c2)c2cnc3c(-c4ccccc4)cc(-c4ccccc4)cc3c2)c1 ; Fc1cccc(-c2ccccc2N(c2ccccc2-c2cccc(F)c2)c2ccnc3c(-c4ccccc4)cc(-c4ccccc4)cc23)c1]

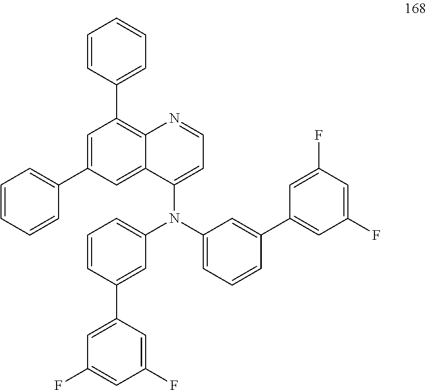 [SMILES: Fc1cc(F)cc(-c2cccc(N(c3cccc(-c4cc(F)cc(F)c4)c3)c3ccnc4c(-c5ccccc5)cc(-c5ccccc5)cc34)c2)c1]

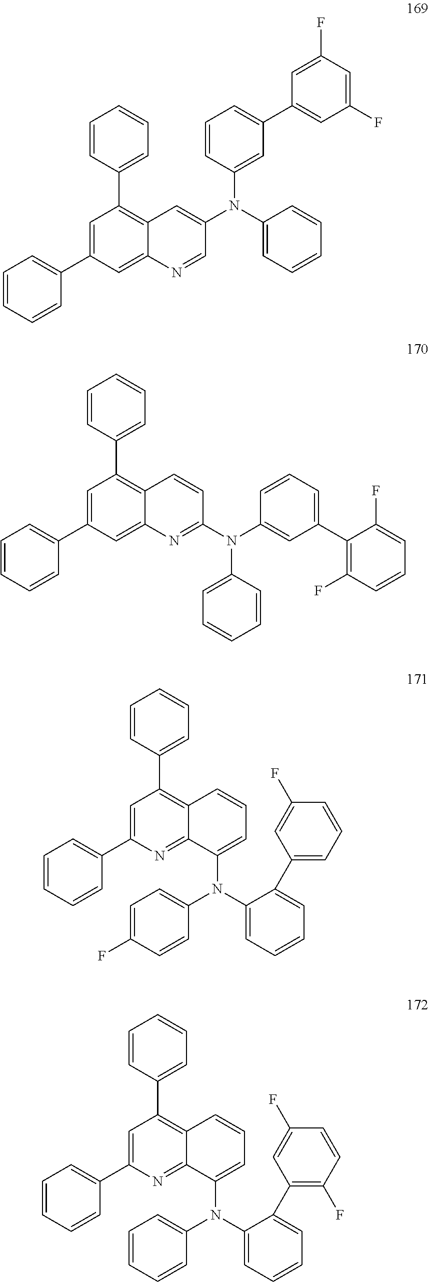 [SMILES: Fc1cc(F)cc(-c2cccc(N(c3ccccc3)c3cnc4cc(-c5ccccc5)cc(-c5ccccc5)c4c3)c2)c1 ; Fc1cccc(F)c1-c1cccc(N(c2ccccc2)c2ccc3c(-c4ccccc4)cc(-c4ccccc4)cc3n2)c1 ; Fc1ccc(N(c2ccccc2-c2cccc(F)c2)c2cccc3c(-c4ccccc4)cc(-c4ccccc4)nc23)cc1 ; Fc1ccc(F)c(-c2ccccc2N(c2ccccc2)c2cccc3c(-c4ccccc4)cc(-c4ccccc4)nc23)c1]

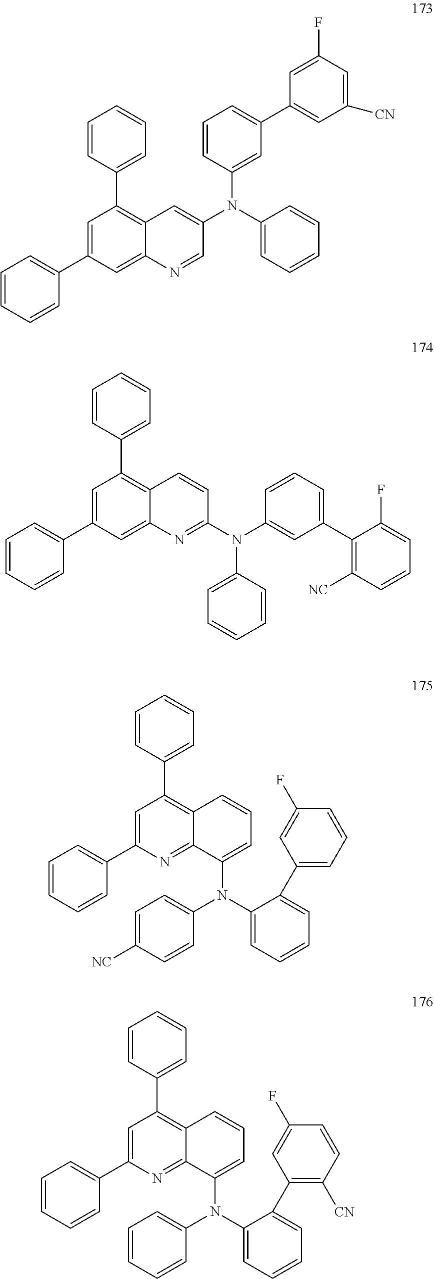 [SMILES: N#Cc1cc(F)cc(-c2cccc(N(c3ccccc3)c3cnc4cc(-c5ccccc5)cc(-c5ccccc5)c4c3)c2)c1 ; N#Cc1cccc(F)c1-c1cccc(N(c2ccccc2)c2ccc3c(-c4ccccc4)cc(-c4ccccc4)cc3n2)c1 ; N#Cc1ccc(N(c2ccccc2-c2cccc(F)c2)c2cccc3c(-c4ccccc4)cc(-c4ccccc4)nc23)cc1 ; N#Cc1ccc(F)cc1-c1ccccc1N(c1ccccc1)c1cccc2c(-c3ccccc3)cc(-c3ccccc3)nc12]

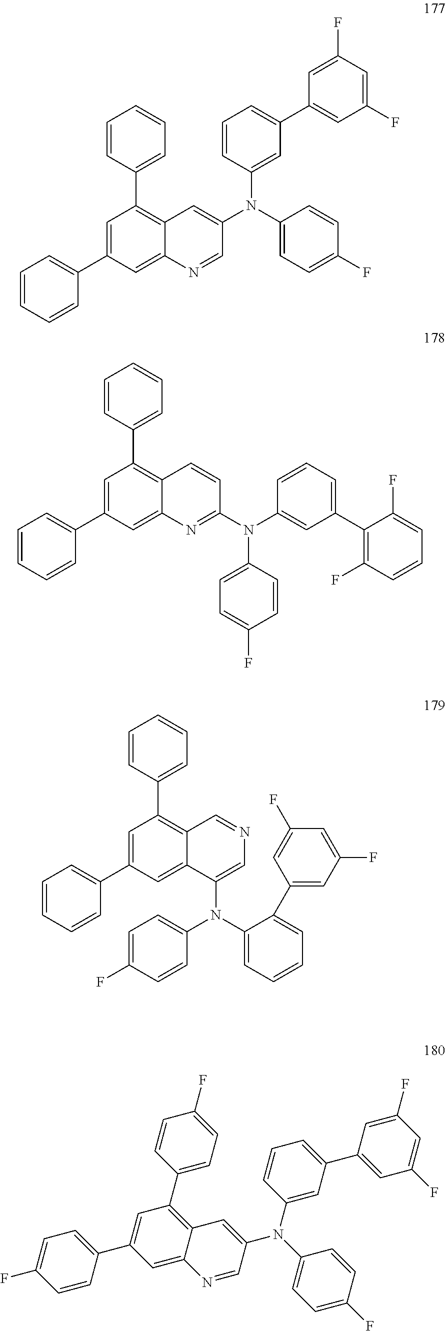 [SMILES: Fc1ccc(N(c2cccc(-c3cc(F)cc(F)c3)c2)c2cnc3cc(-c4ccccc4)cc(-c4ccccc4)c3c2)cc1 ; Fc1ccc(N(c2cccc(-c3c(F)cccc3F)c2)c2ccc3c(-c4ccccc4)cc(-c4ccccc4)cc3n2)cc1 ; Fc1ccc(N(c2ccccc2-c2cc(F)cc(F)c2)c2cncc3c(-c4ccccc4)cc(-c4ccccc4)cc23)cc1 ; Fc1ccc(-c2cc(-c3ccc(F)cc3)c3cc(N(c4ccc(F)cc4)c4cccc(-c5cc(F)cc(F)c5)c4)cnc3c2)cc1]

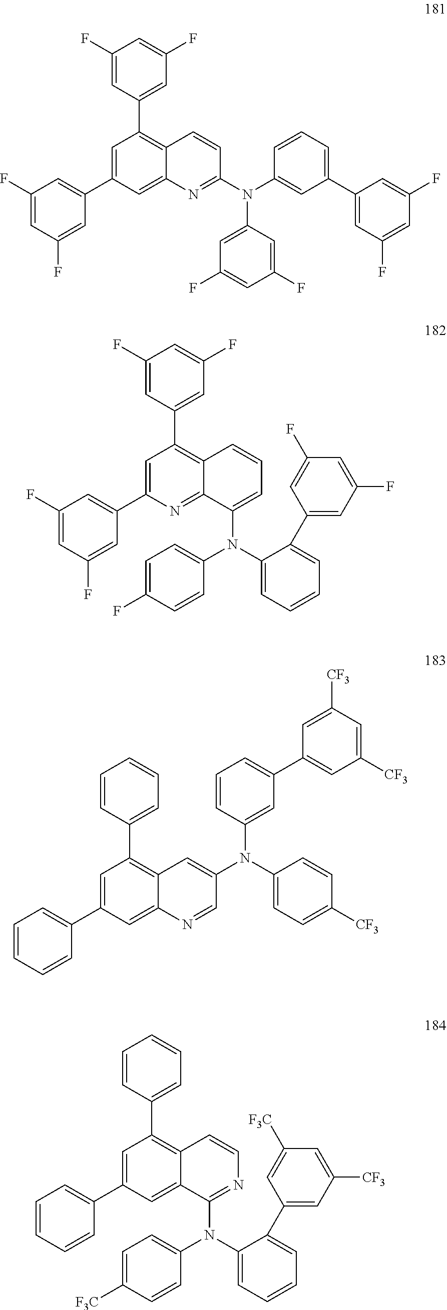 [SMILES: Fc1cc(F)cc(-c2cccc(N(c3cc(F)cc(F)c3)c3ccc4c(-c5cc(F)cc(F)c5)cc(-c5cc(F)cc(F)c5)cc4n3)c2)c1 ; Fc1ccc(N(c2ccccc2-c2cc(F)cc(F)c2)c2cccc3c(-c4cc(F)cc(F)c4)cc(-c4cc(F)cc(F)c4)nc23)cc1 ; FC(F)(F)c1ccc(N(c2cccc(-c3cc(C(F)(F)F)cc(C(F)(F)F)c3)c2)c2cnc3cc(-c4ccccc4)cc(-c4ccccc4)c3c2)cc1 ; FC(F)(F)c1ccc(N(c2ccccc2-c2cc(C(F)(F)F)cc(C(F)(F)F)c2)c2nccc3c(-c4ccccc4)cc(-c4ccccc4)cc23)cc1]

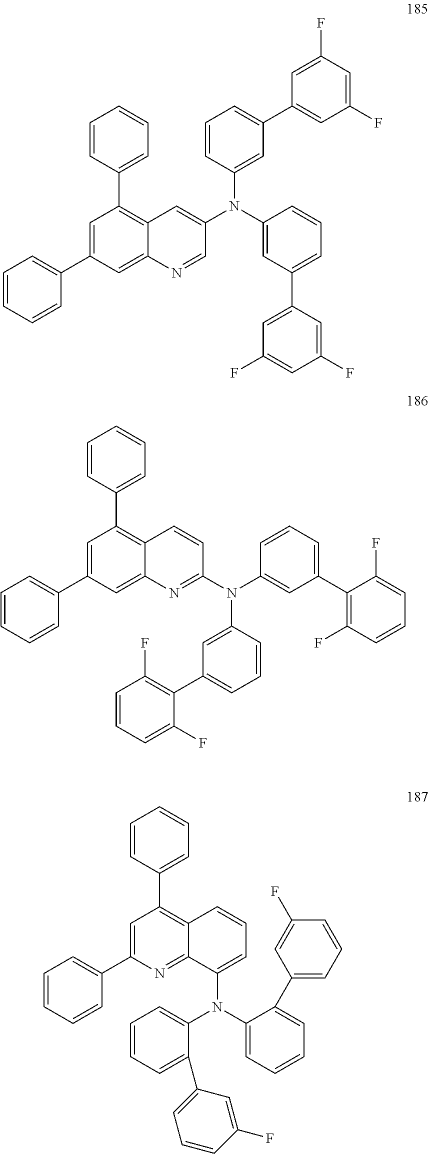 [SMILES: Fc1cc(F)cc(-c2cccc(N(c3cccc(-c4cc(F)cc(F)c4)c3)c3cnc4cc(-c5ccccc5)cc(-c5ccccc5)c4c3)c2)c1 ; Fc1cccc(F)c1-c1cccc(N(c2cccc(-c3c(F)cccc3F)c2)c2ccc3c(-c4ccccc4)cc(-c4ccccc4)cc3n2)c1 ; Fc1cccc(-c2ccccc2N(c2ccccc2-c2cccc(F)c2)c2cccc3c(-c4ccccc4)cc(-c4ccccc4)nc23)c1]

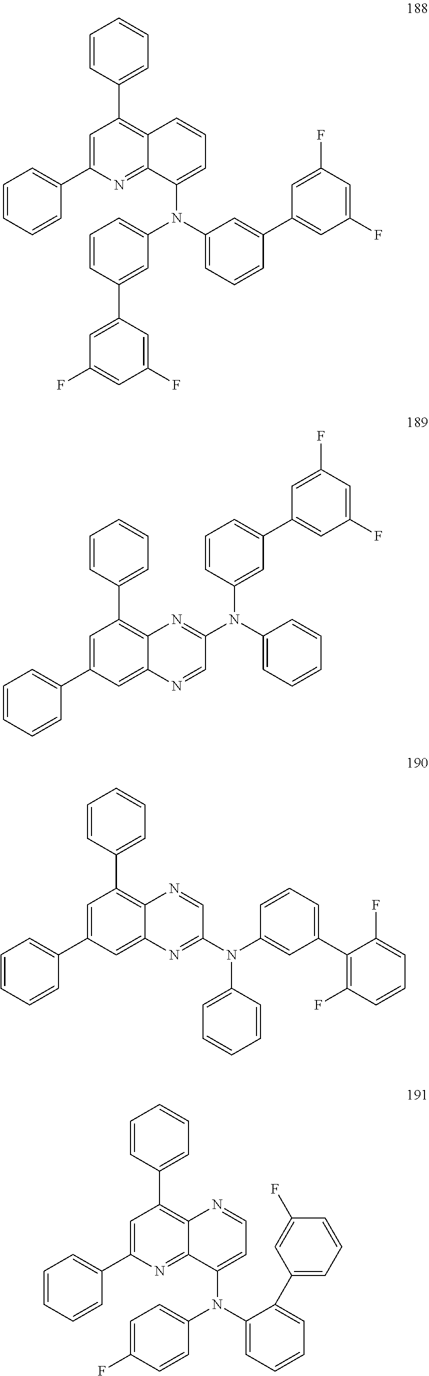 [SMILES: Fc1cc(F)cc(-c2cccc(N(c3cccc(-c4cc(F)cc(F)c4)c3)c3cccc4c(-c5ccccc5)cc(-c5ccccc5)nc34)c2)c1 ; Fc1cc(F)cc(-c2cccc(N(c3ccccc3)c3cnc4cc(-c5ccccc5)cc(-c5ccccc5)c4n3)c2)c1 ; Fc1cccc(F)c1-c1cccc(N(c2ccccc2)c2cnc3c(-c4ccccc4)cc(-c4ccccc4)cc3n2)c1 ; Fc1ccc(N(c2ccccc2-c2cccc(F)c2)c2ccnc3c(-c4ccccc4)cc(-c4ccccc4)nc23)cc1]

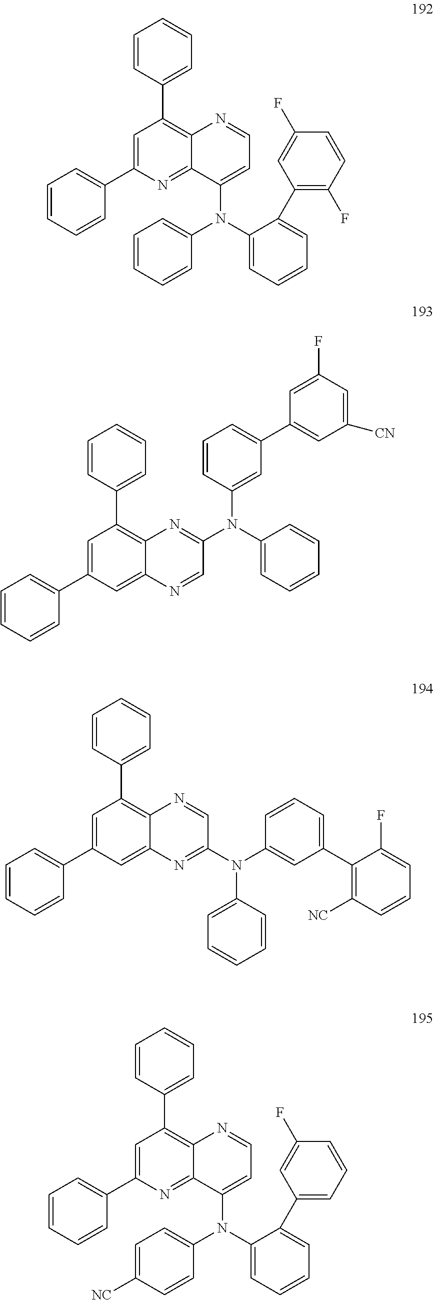 [SMILES: Fc1ccc(F)c(-c2ccccc2N(c2ccccc2)c2ccnc3c(-c4ccccc4)cc(-c4ccccc4)nc23)c1 ; N#Cc1cc(F)cc(-c2cccc(N(c3ccccc3)c3cnc4cc(-c5ccccc5)cc(-c5ccccc5)c4n3)c2)c1 ; N#Cc1cccc(F)c1-c1cccc(N(c2ccccc2)c2cnc3c(-c4ccccc4)cc(-c4ccccc4)cc3n2)c1 ; N#Cc1ccc(N(c2ccccc2-c2cccc(F)c2)c2ccnc3c(-c4ccccc4)cc(-c4ccccc4)nc23)cc1]

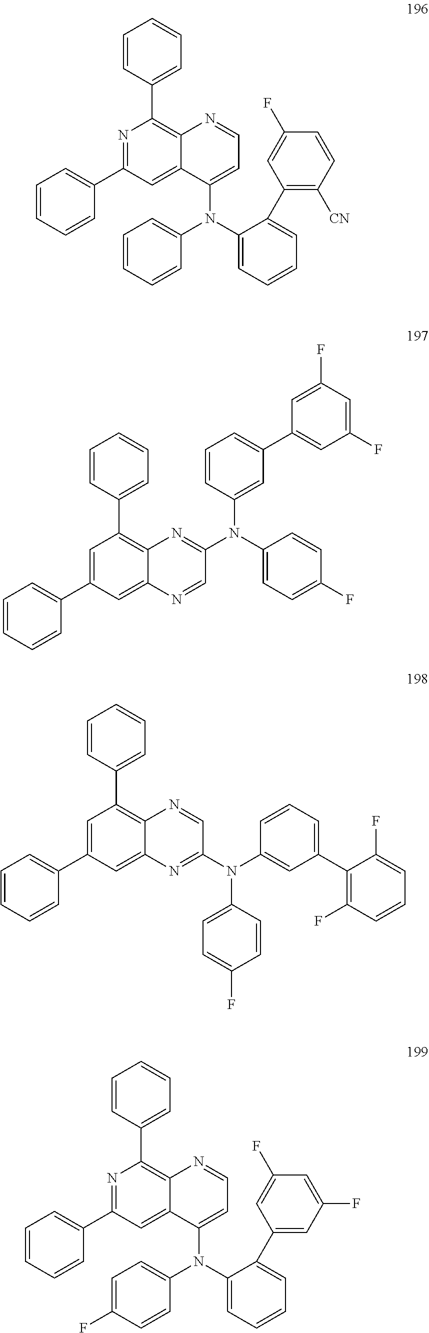 [SMILES: N#Cc1ccc(F)cc1-c1ccccc1N(c1ccccc1)c1ccnc2c(-c3ccccc3)nc(-c3ccccc3)cc12 ; Fc1ccc(N(c2cccc(-c3cc(F)cc(F)c3)c2)c2cnc3cc(-c4ccccc4)cc(-c4ccccc4)c3n2)cc1 ; Fc1ccc(N(c2cccc(-c3c(F)cccc3F)c2)c2cnc3c(-c4ccccc4)cc(-c4ccccc4)cc3n2)cc1 ; Fc1ccc(N(c2ccccc2-c2cc(F)cc(F)c2)c2ccnc3c(-c4ccccc4)nc(-c4ccccc4)cc23)cc1]

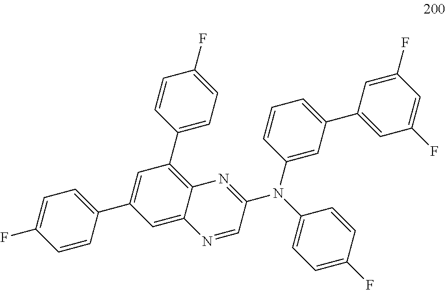 [SMILES: Fc1ccc(-c2cc(-c3ccc(F)cc3)c3nc(N(c4ccc(F)cc4)c4cccc(-c5cc(F)cc(F)c5)c4)cnc3c2)cc1]

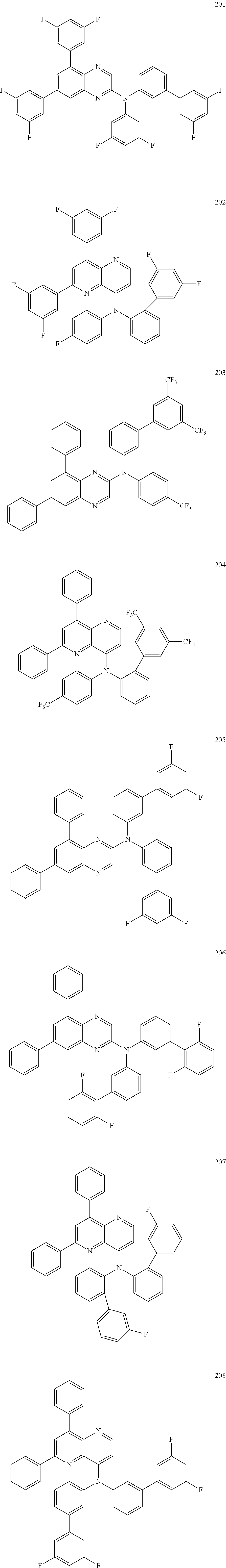

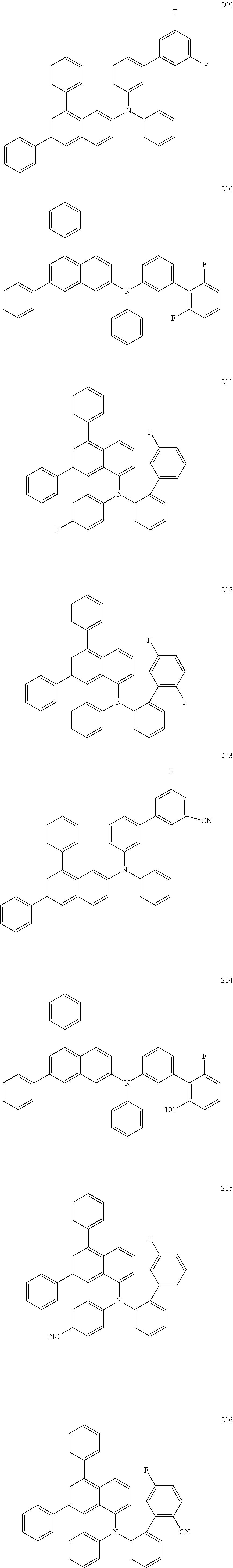

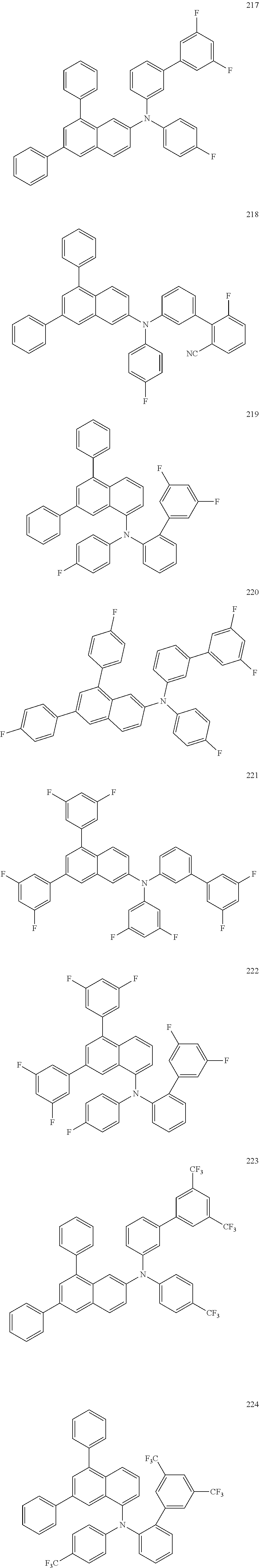

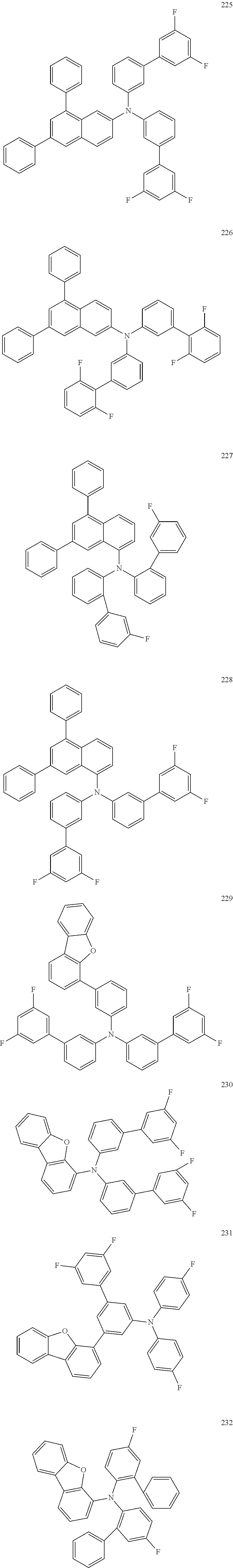

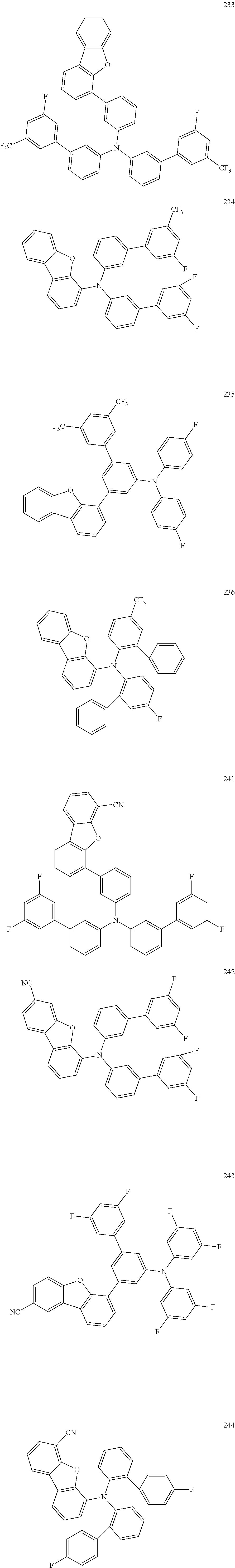

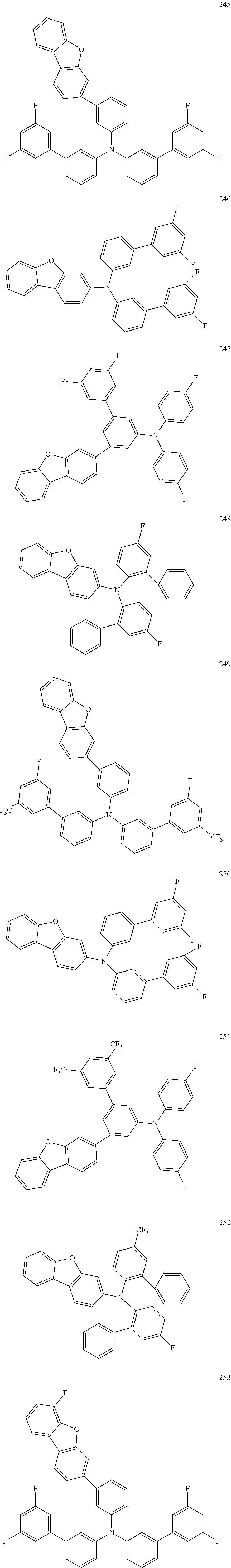

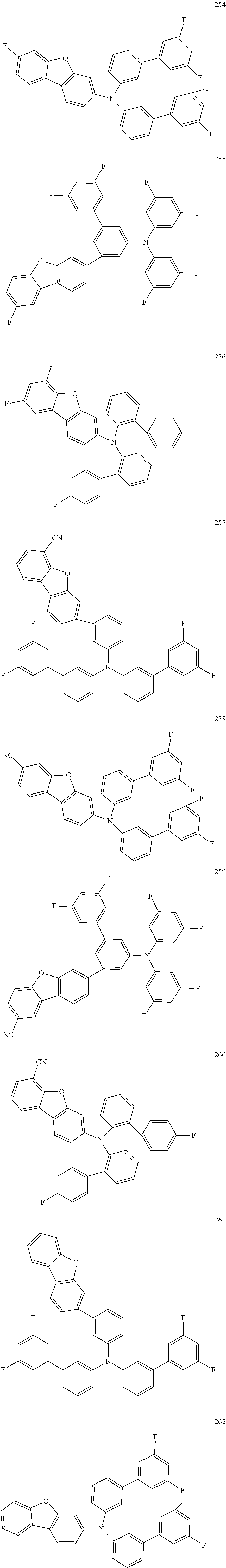

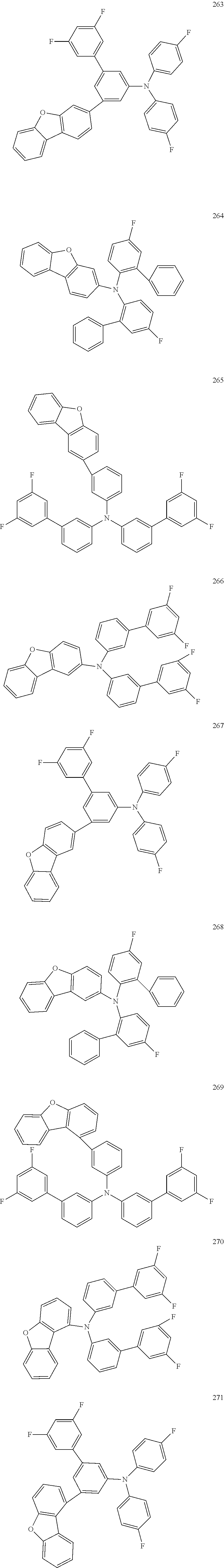

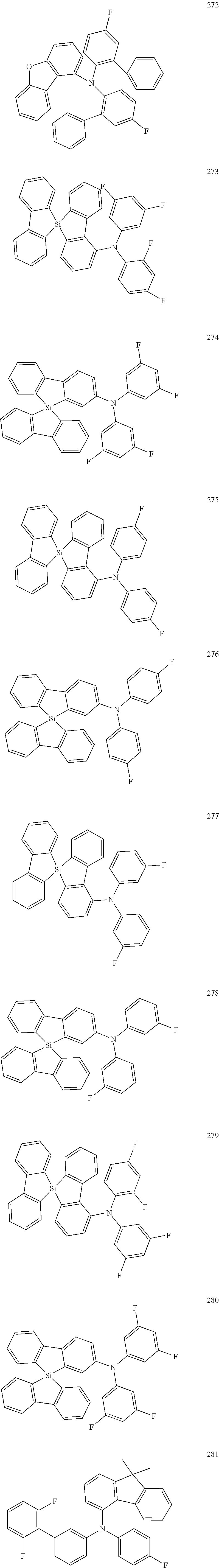

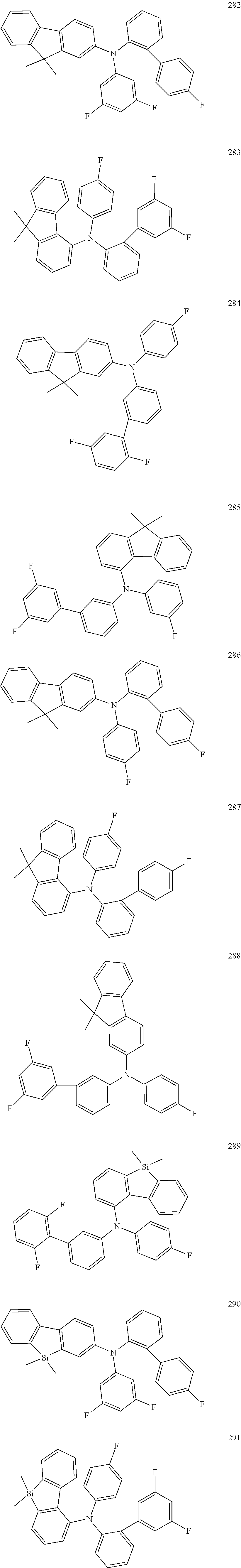

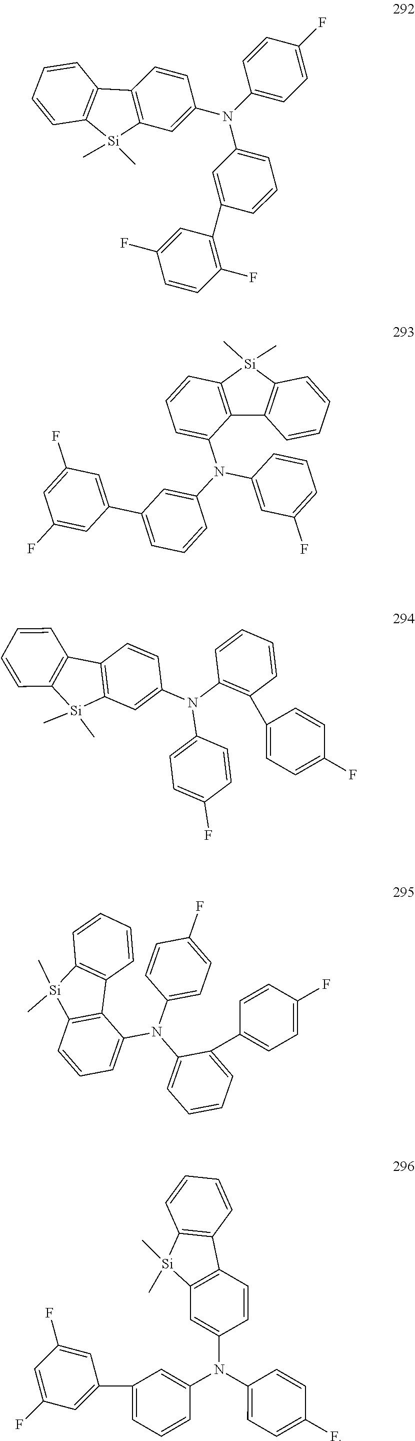 [SMILES: C[Si]1(C)c2ccccc2-c2ccc(N(c3ccc(F)cc3)c3cccc(-c4cc(F)ccc4F)c3)cc21 ; C[Si]1(C)c2ccccc2-c2c(N(c3cccc(F)c3)c3cccc(-c4cc(F)cc(F)c4)c3)cccc21 ; C[Si]1(C)c2ccccc2-c2ccc(N(c3ccc(F)cc3)c3ccccc3-c3ccc(F)cc3)cc21 ; C[Si]1(C)c2ccccc2-c2c(N(c3ccc(F)cc3)c3ccccc3-c3ccc(F)cc3)cccc21 ; C[Si]1(C)c2ccccc2-c2ccc(N(c3ccc(F)cc3)c3cccc(-c4cc(F)cc(F)c4)c3)cc21]

A second object of the present disclosure is to provide an organic electroluminescent material including the organic compound as described in the first object.

A third object of the present disclosure is to provide an organic capping layer material including the organic compound as described in the first object.

A fourth object of the present disclosure is to provide an OLED device. The OLED device includes an anode, a cathode, and an organic thin-film layer disposed between the anode and the cathode, where the material of the organic thin-film layer includes the organic compound as described in the first object.

A fifth object of the present disclosure is to provide an OLED device. The OLED includes an anode, a cathode, an organic thin-film layer disposed between the anode and the cathode, and an organic capping layer disposed above the cathode, where the material of the organic capping layer includes the organic compound as described in the first object.

In one embodiment, the organic capping layer includes a first capping layer and a second capping layer, the first capping layer includes the organic compound as described in the first object, and the second capping layer includes a high-refractive-index material layer having a refractive index greater than or equal to 2.2.

In one embodiment, the high-refractive-index material layer includes any one of the following materials:

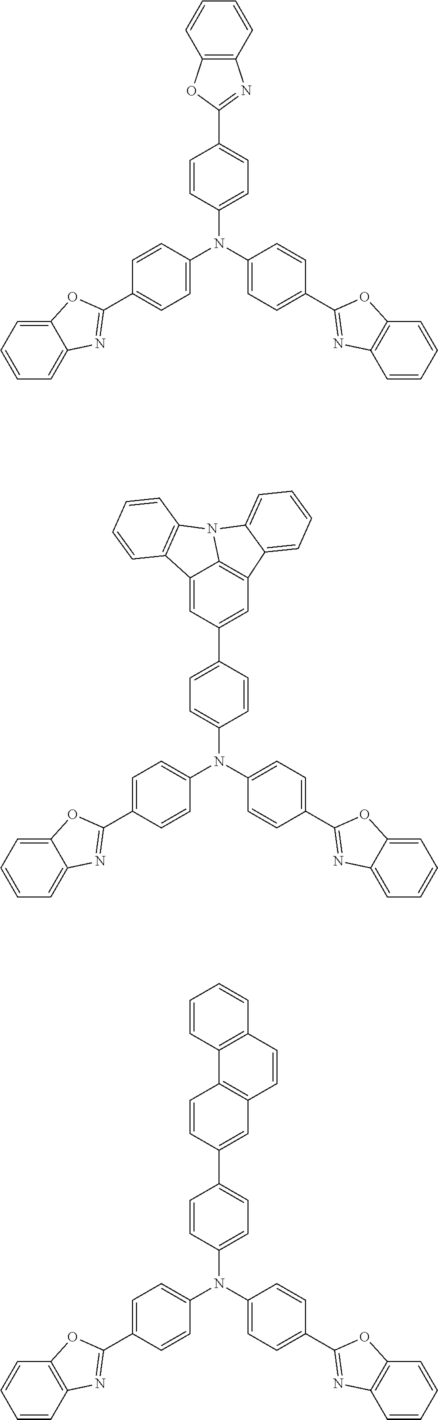 [SMILES: c1ccc2oc(-c3ccc(N(c4ccc(-c5nc6ccccc6o5)cc4)c4ccc(-c5nc6ccccc6o5)cc4)cc3)nc2c1 ; c1ccc2oc(-c3ccc(N(c4ccc(-c5cc6c7ccccc7n7c8ccccc8c(c5)c67)cc4)c4ccc(-c5nc6ccccc6o5)cc4)cc3)nc2c1 ; c1ccc2c(c1)ccc1cc(-c3ccc(N(c4ccc(-c5nc6ccccc6o5)cc4)c4ccc(-c5nc6ccccc6o5)cc4)cc3)ccc12]

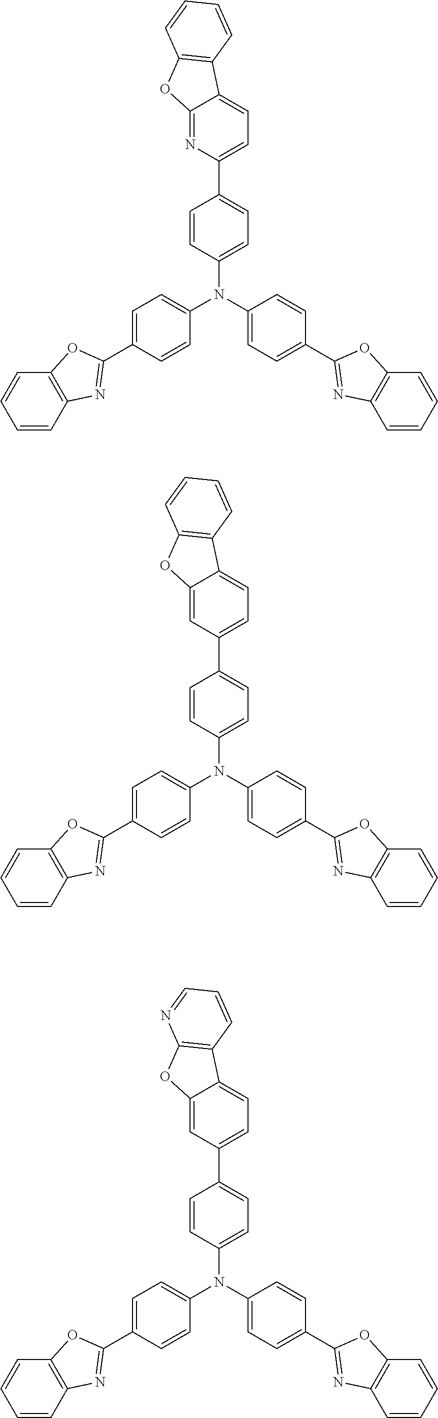 [SMILES: c1ccc2oc(-c3ccc(N(c4ccc(-c5ccc6c(n5)oc5ccccc56)cc4)c4ccc(-c5nc6ccccc6o5)cc4)cc3)nc2c1 ; c1ccc2oc(-c3ccc(N(c4ccc(-c5ccc6c(c5)oc5ccccc56)cc4)c4ccc(-c5nc6ccccc6o5)cc4)cc3)nc2c1 ; c1ccc2oc(-c3ccc(N(c4ccc(-c5ccc6c(c5)oc5ncccc56)cc4)c4ccc(-c5nc6ccccc6o5)cc4)cc3)nc2c1]

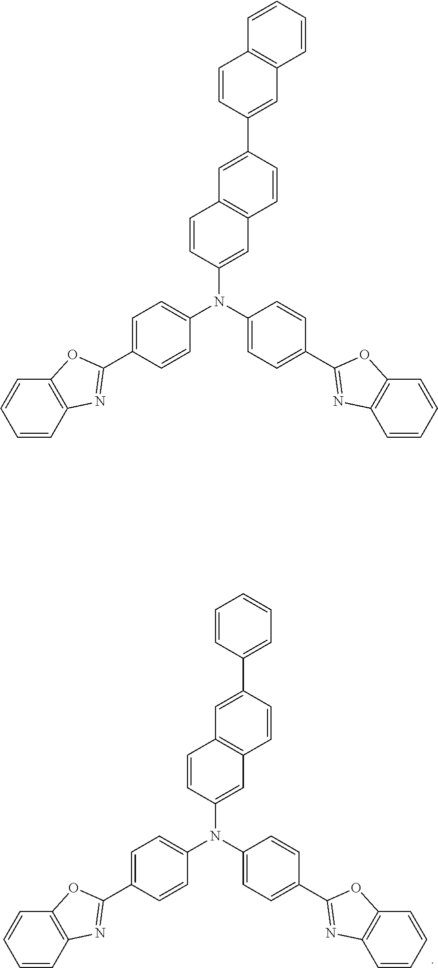 [SMILES: c1ccc2cc(-c3ccc4cc(N(c5ccc(-c6nc7ccccc7o6)cc5)c5ccc(-c6nc7ccccc7o6)cc5)ccc4c3)ccc2c1 ; c1ccc(-c2ccc3cc(N(c4ccc(-c5nc6ccccc6o5)cc4)c4ccc(-c5nc6ccccc6o5)cc4)ccc3c2)cc1]

A sixth object of the present disclosure is to provide a display panel including the OLED device as described in the fourth or fifth object.

A seventh object of the present disclosure is to provide an organic light-emitting display device including the display panel as described in the sixth object.

An eighth object of the present disclosure is to provide an electronic device including the display panel as described in the sixth object.

The representative synthesis route of the compound of Formula I provided by the present disclosure is as follows:

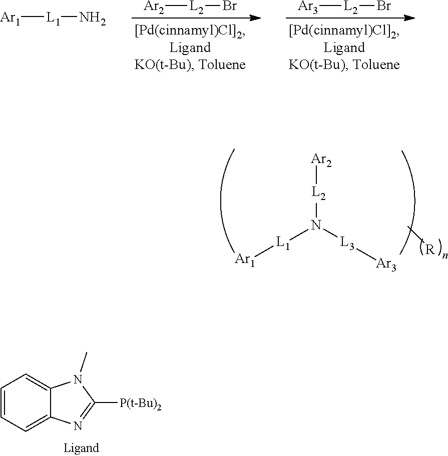

where Toluene represents toluene, KO(t-Bu) represents potassium tert-butoxide, [Pd(cinnamyl)Cl]₂ represents palladium(1-phenylallyl)chloride, Ligand represents a ligand, and t-Bu represents t-butyl.

The specific synthesis methods for a series of compounds are exemplified in the following examples. Compounds whose specific synthesis methods are not mentioned may be synthesized by similar methods or other existing methods, which are not specifically limited in the present disclosure.

### EXAMPLE 1

The synthesis route of Compound 1 is as follows:

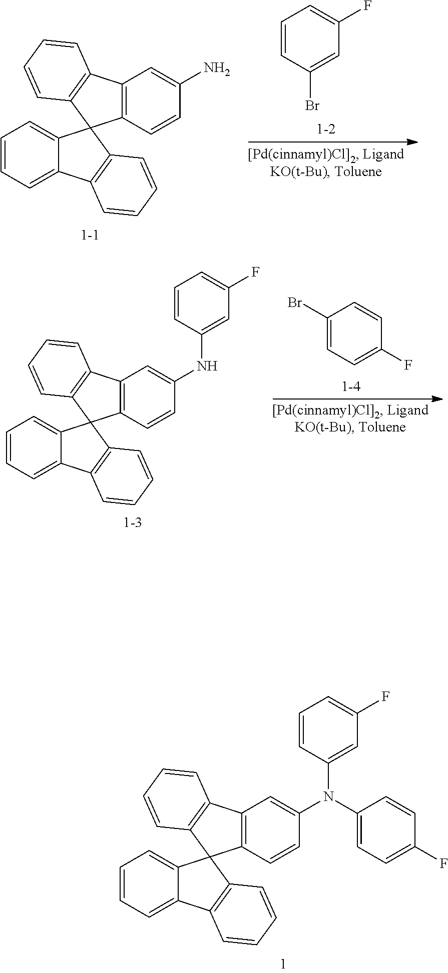 [SMILES: Nc1ccc2c(c1)-c1ccccc1C21c2ccccc2-c2ccccc21 ; Fc1cccc(Br)c1 ; Fc1cccc(Nc2ccc3c(c2)-c2ccccc2C32c3ccccc3-c3ccccc32)c1 ; Fc1ccc(Br)cc1 ; Fc1ccc(N(c2cccc(F)c2)c2ccc3c(c2)-c2ccccc2C32c3ccccc3-c3ccccc32)cc1]

(1) 1-1 (0.5 mmol), 1-2 (0.45 mmol), KO(t-Bu) (0.5 mmol), [Pd(cinnamyl)Cl]₂ (1 mol %) and Ligand (1.0 mol %) were added to 3 mL of toluene solution, mixed, placed in a 50 mL flask, and reacted at 60° C. for 12 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 1-3.

The structure of the target product 1-3 was tested through matrix-assisted laser desorption/ionization time-of-flight mass spectrometry (MALDI-TOF-MS) (m/z): C₃₁H₂₀FN whose calculated value was 425.5 and whose measured value was 425.2.

(2) 1-3 (0.5 mmol), 1-4 (0.65 mmol), KO(t-Bu) (0.65 mmol), [Pd(cinnamyl)Cl]₂ (1.5 mol %) and Ligand (1.5 mol %) were added to 3 mL of toluene solution, mixed, placed in a 50 mL flask, and reacted at 80° C. for 12 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 1.

The structure of the target product 1 was tested through matrix-assisted laser desorption/ionization time-of-flight mass spectrometry (MALDI-TOF-MS) (m/z): C₃₇H₂₃F₂N whose calculated value was 519.6 and whose measured value was 519.2.

Elemental analysis: theoretical value: C, 85.53; H, 4.46; N, 2.70; measured value: C, 85.52; H, 4.46; N, 2.71.

### EXAMPLE 2

The synthesis route of Compound 4 is as follows:

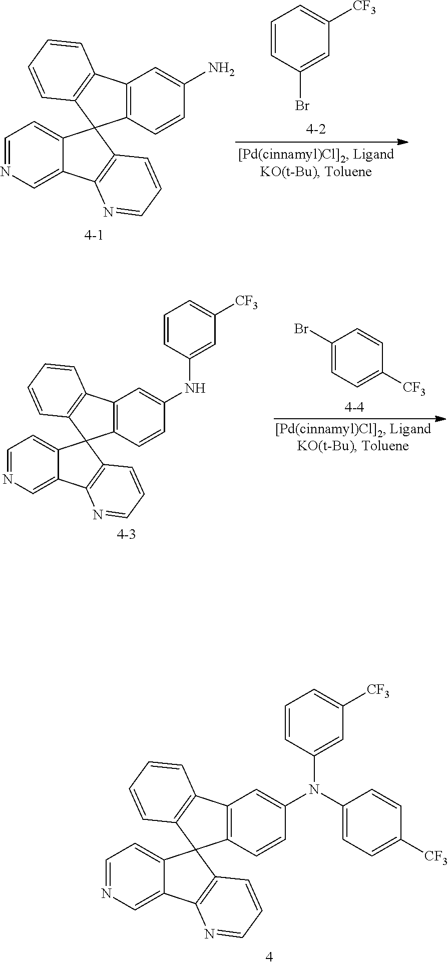 [SMILES: Nc1ccc2c(c1)-c1ccccc1C21c2ccncc2-c2ncccc21 ; FC(F)(F)c1cccc(Br)c1 ; FC(F)(F)c1cccc(Nc2ccc3c(c2)-c2ccccc2C32c3ccncc3-c3ncccc32)c1 ; FC(F)(F)c1ccc(Br)cc1 ; FC(F)(F)c1ccc(N(c2cccc(C(F)(F)F)c2)c2ccc3c(c2)-c2ccccc2C32c3ccncc3-c3ncccc32)cc1]

(1) 4-1 (0.5 mmol), 4-2 (0.45 mmol), KO(t-Bu) (0.5 mmol), [Pd(cinnamyl)Cl]2 (1 mol %) and Ligand (1.0 mol %) were added to 3 mL of toluene solution, mixed, placed in a 50 mL flask, and reacted at 50° C. for 16 hours. After the solution was cooled to room temperature, saturated aqueous MgSO4 solution and ethyl acetate were slowly added to the solution for extraction three times, and the organic layer was then processed by a rotary evaporator to remove the solvent and processed by column chromatography to give a crude product 4-3.

The structure of the target product 4-3 was tested through matrix-assisted laser desorption/ionization time-of-flight mass spectrometry (MALDI-TOF-MS) (m/z): C₃₀H₁₈F₃N₃ whose calculated value was 477.5 and whose measured value was 477.2.

(2) 4-3 (0.5 mmol), 4-4 (0.65 mmol), KO(t-Bu) (0.65 mmol), [Pd(cinnamyl)Cl]₂ (1.5 mol %) and Ligand (1.5 mol %) were added to 3 mL of toluene solution, mixed, placed in a 50 mL flask, and reacted at 75° C. for 14 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 4.

The structure of the target product 4 was tested through matrix-assisted laser desorption/ionization time-of-flight mass spectrometry (MALDI-TOF-MS) (m/z): C₃₇H₂₁F₆N₃ whose calculated value was 621.6 and whose measured value was 621.2.

Elemental analysis: theoretical value: C, 71.50; H, 3.41; N, 6.76; measured value: C, 71.50; H, 3.40; N, 6.78.

### EXAMPLE 3

The synthesis route of Compound 10 is as follows:

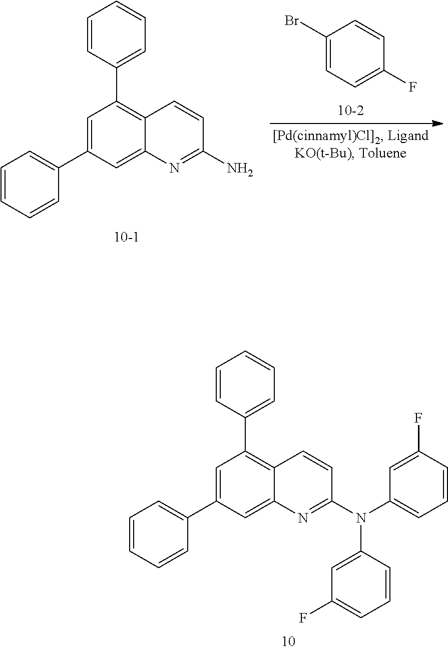 [SMILES: Nc1ccc2c(-c3ccccc3)cc(-c3ccccc3)cc2n1 ; Fc1ccc(Br)cc1 ; Fc1cccc(N(c2cccc(F)c2)c2ccc3c(-c4ccccc4)cc(-c4ccccc4)cc3n2)c1]

(1) 10-1 (0.5 mmol), 10-2 (1.3 mmol), KO(t-Bu) (1.1 mmol), [Pd(cinnamyl)Cl]₂ (2.0 mol %) and Ligand (2.0 mol %) were added to 3 mL of toluene solution, mixed, placed in a 50 mL flask, and reacted at 80° C. for 14 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 10.

The structure of the target product 10 was tested through matrix-assisted laser desorption/ionization time-of-flight mass spectrometry (MALDI-TOF-MS) (m/z): C₃₃H₂₂F₂N₂ whose calculated value was 484.6 and whose measured value was 484.2.

Elemental analysis: theoretical value: C, 81.80; H, 4.58; N, 5.78; measured value: C, 81.80; H, 4.59; N, 5.79.

### EXAMPLE 4

The synthesis route of Compound 15 is as follows:

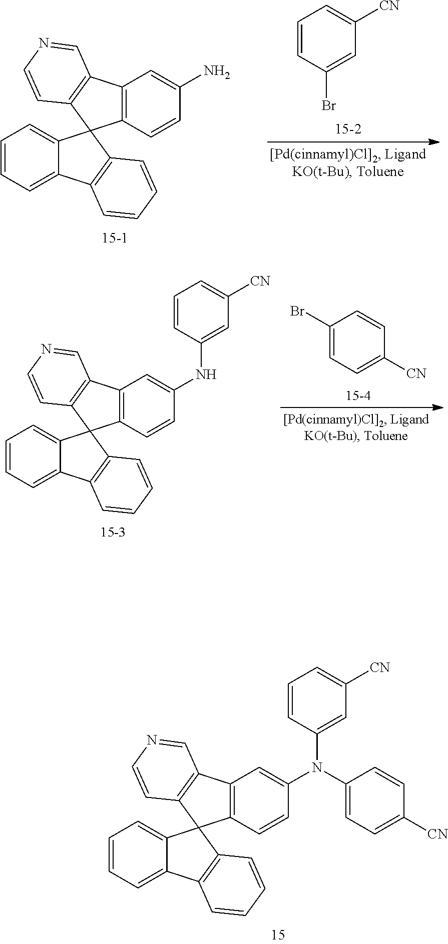 [SMILES: Nc1ccc2c(c1)-c1cnccc1C21c2ccccc2-c2ccccc21 ; N#Cc1cccc(Br)c1 ; N#Cc1cccc(Nc2ccc3c(c2)-c2cnccc2C32c3ccccc3-c3ccccc32)c1 ; N#Cc1ccc(Br)cc1 ; N#Cc1ccc(N(c2cccc(C#N)c2)c2ccc3c(c2)-c2cnccc2C32c3ccccc3-c3ccccc32)cc1]

(1) 15-1 (0.5 mmol), 15-2 (0.45 mmol), KO(t-Bu) (0.5 mmol), [Pd(cinnamyl)Cl]₂ (1 mol %) and Ligand (1.0 mol %) were added to 3 mL of toluene solution, mixed, placed in a 50 mL flask, and reacted at 50° C. for 16 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 15-3.

The structure of the target product 15-3 was tested through matrix-assisted laser desorption/ionization time-of-flight mass spectrometry (MALDI-TOF-MS) (m/z): C₃₁H₁₉N₃ whose calculated value was 433.5 and whose measured value was 433.2.

(2) 15-3 (0.5 mmol), 15-4 (0.65 mmol), KO(t-Bu) (0.65 mmol), [Pd(cinnamyl)Cl]₂ (1.5 mol %) and Ligand (1.5 mol %) were added to 3 mL of toluene solution, mixed, placed in a 50 mL flask, and reacted at 75° C. for 14 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 15.

The structure of the target product 15 was tested through matrix-assisted laser desorption/ionization time-of-flight mass spectrometry (MALDI-TOF-MS) (m/z): C₃₈H₂₂N₄ whose calculated value was 534.6 and whose measured value was 534.2.

Elemental analysis: theoretical value: C, 85.37; H, 4.15; N, 10.48; measured value: C, 85.37; H, 4.16; N, 10.49.

### EXAMPLE 5

The synthesis route of Compound 273 is as follows:

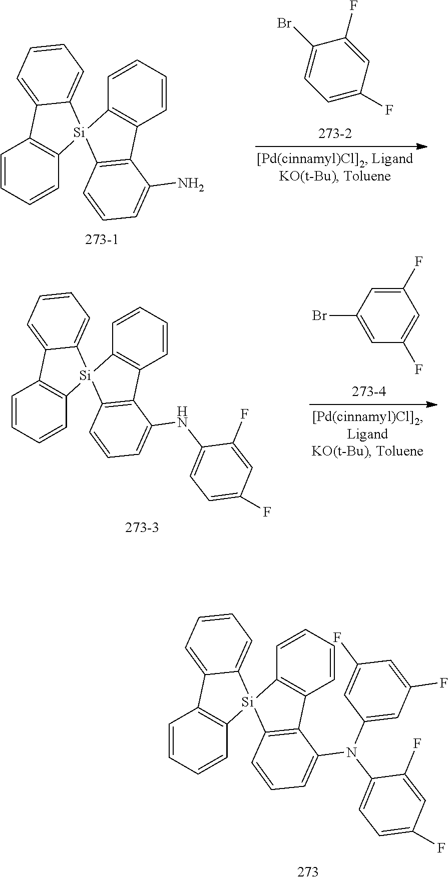 [SMILES: Nc1cccc2c1-c1ccccc1[Si]21c2ccccc2-c2ccccc21 ; Fc1ccc(Br)c(F)c1 ; Fc1ccc(Nc2cccc3c2-c2ccccc2[Si]32c3ccccc3-c3ccccc32)c(F)c1 ; Fc1cc(F)cc(Br)c1 ; Fc1cc(F)cc(N(c2ccc(F)cc2F)c2cccc3c2-c2ccccc2[Si]32c3ccccc3-c3ccccc32)c1]

(1) 273-1 (0.5 mmol), 273-2 (0.45 mmol), KO(t-Bu) (0.5 mmol), [Pd(cinnamyl)C1]₂ (1 mol %) and Ligand (1.0 mol %) were added to 3 mL of toluene solution, mixed, placed in a 50 mL flask, and reacted at 50° C. for 16 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 273-3.

The structure of the target product 273-3 was tested through matrix-assisted laser desorption/ionization time-of-flight mass spectrometry (MALDI-TOF-MS) (m/z): C₃₀H₁₉F₂NSi whose calculated value was 459.6 and whose measured value was 459.1.

(2) 273-3 (0.5 mmol), 273-4 (0.65 mmol), KO(t-Bu) (0.65 mmol), [Pd(cinnamyl)Cl]₂ (1.5 mol %) and Ligand (1.5 mol %) were added to 3 mL of toluene solution, mixed, placed in a 50 mL flask, and reacted at 75° C. for 14 hours. After the solution was cooled to room temperature, saturated aqueous MgSO₄ solution and ethyl acetate were slowly added to the solution for extraction three times, and the organic layer was then processed by a rotary evaporator to remove the solvent and processed by column chromatography to give a crude product 273.

The structure of the target product 273 was tested through matrix-assisted laser desorption/ionization time-of-flight mass spectrometry (MALDI-TOF-MS) (m/z): C₃₆H₂₁F₄NSi whose calculated value was 571.7 and whose measured value was 571.1.

Elemental analysis: theoretical value: C, 75.64; H, 3.70; N, 2.45; measured value: C, 75.64; H, 3.71; N, 2.46.

The preparation methods for other compounds of the present disclosure are all similar to the methods described above and thus are not repeated here, and only the characterization results of mass spectrometry and elemental analysis of some other compounds of the present disclosure are provided, as shown in Table 1.

TABLE 1
Compound characterization result

| | Mass spectrometry | | | |
|---|---|---|---|---|
| | result | | | |
| | Calculated | Measured | Elemental analysis result | |
| Compound | value | value | Calculated value | Measured value |
| 2 | 619.6 | 619.2 | C, 75.60; H, 3.74; N, 2.26; | C, 75.60; H, 3.74; N, 2.27; |
| 3 | 520.6 | 520.2 | C, 83.06; H, 4.26; N, 5.38; | C, 83.06; H, 4.26; N, 5.39; |
| 7 | 485.5 | 485.2 | C, 79.16; H, 4.36; N, 8.65; | C, 79.16; H, 4.37; N, 8.65; |
| 11 | 541.6 | 541.2 | C, 79.84; H, 4.09; N, 2.59; | C, 79.83; H, 4.08; N, 2.59; |
| 33 | 555.6 | 555.2 | C, 79.99; H, 3.81; N, 2.52; | C, 79.99; H, 3.80; N, 2.52; |
| 41 | 755.6 | 755.2 | C, 65.17; H, 2.80; N, 1.85; | C, 65.17; H, 2.80; N, 1.86; |
| 109 | 669.6 | 669.2 | C, 68.17; H, 2.86; N, 6.28; | C, 68.17; H, 2.87; N, 6.28; |
| 230 | 559.6 | 559.2 | C, 77.27; H, 3.78; N, 2.50; | C, 77.28; H, 3.79; N, 2.50; |
| 285 | 491.6 | 491.2 | C, 80.63; H, 4.92; N, 2.85; | C, 80.63; H, 4.93; N, 2.84; |
| 295 | 489.6 | 489.2 | C, 78.50; H, 5.15; N, 2.86; | C, 78.50; H, 5.15; N, 2.87; |

### Performance Test: Material Refractive Index Characterization

The refractive indexes of the compounds at wavelengths of 460 nm, 530 nm and 620 nm were tested by an ellipsometer. The difference Δn₁ between the refractive index at the wavelength of 460 nm and the refractive index at the wavelength of 530 nm, the difference Δn₂ between the refractive index at the wavelength of 530 nm and the refractive index at the wavelength of 620 nm, and the difference Δn₃ between the refractive index at the wavelength of 460 nm and the refractive index at the wavelength of 620 nm were calculated.

The results of the preceding test are shown in Table 2.

TABLE 2

| Compound | n_{460 nm} | n_{530 nm} | n_{620 nm} | Δn₁ | Δn₂ | Δn₃ |
|---|---|---|---|---|---|---|
| 1 | 1.77 | 1.72 | 1.70 | 0.05 | 0.02 | 0.07 |
| 2 | 1.73 | 1.67 | 1.64 | 0.06 | 0.03 | 0.09 |
| 3 | 1.76 | 1.73 | 1.69 | 0.03 | 0.04 | 0.07 |
| 4 | 1.72 | 1.65 | 1.62 | 0.07 | 0.03 | 0.10 |
| 7 | 1.76 | 1.72 | 1.70 | 0.04 | 0.02 | 0.06 |
| 10 | 1.73 | 1.68 | 1.66 | 0.05 | 0.02 | 0.07 |
| 11 | 1.72 | 1.67 | 1.63 | 0.05 | 0.04 | 0.09 |
| 15 | 1.77 | 1.74 | 1.70 | 0.03 | 0.04 | 0.07 |
| 33 | 1.74 | 1.69 | 1.67 | 0.05 | 0.02 | 0.07 |
| 41 | 1.68 | 1.64 | 1.63 | 0.04 | 0.01 | 0.05 |
| 109 | 1.70 | 1.66 | 1.60 | 0.04 | 0.06 | 0.10 |
| 230 | 1.69 | 1.65 | 1.61 | 0.04 | 0.04 | 0.08 |
| 273 | 1.73 | 1.69 | 1.66 | 0.04 | 0.03 | 0.07 |
| 285 | 1.70 | 1.66 | 1.64 | 0.04 | 0.02 | 0.06 |
| 295 | 1.71 | 1.67 | 1.63 | 0.04 | 0.04 | 0.08 |
| C1 | 2.10 | 1.99 | 1.89 | 0.12 | 0.10 | 0.22 |
| C2 | 2.20 | 2.06 | 1.96 | 0.14 | 0.10 | 0.24 |
| C3 | 2.18 | 2.05 | 1.95 | 0.13 | 0.10 | 0.23 |
| C4 | 2.08 | 1.96 | 1.87 | 0.12 | 0.09 | 0.21 |
| C5 | 2.06 | 1.95 | 1.86 | 0.11 | 0.09 | 0.20 |

Comparative Compounds C1, C2 and C3 have the following structures:

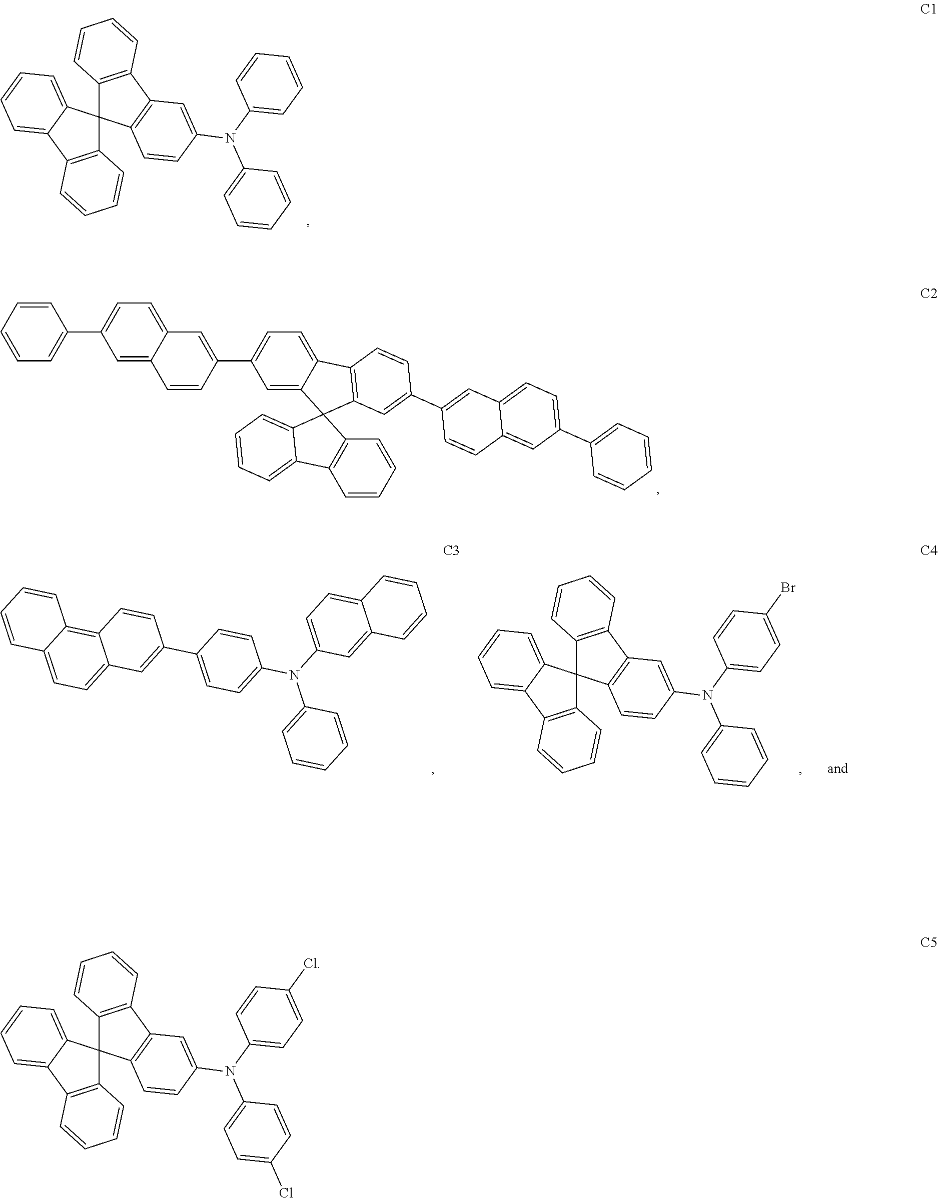

As can be seen from Table 2, the compounds provided by the present disclosure meet the requirement that the difference between the refractive index at the wavelength of 460 nm and the refractive index at the wavelength of 530 nm is 0.03-0.07, the difference between the refractive index at the wavelength of 530 nm and the refractive index at the wavelength of 620 nm is 0.01-0.06, and the difference between the refractive index at the wavelength of 460 nm and the refractive index at the wavelength of 620 nm is 0.05-0.10. When the compounds provided by the present disclosure are used in the organic electroluminescent devices, since Δn is small and the difference between the refractive indexes at the different light colors is small, the compounds provided by the present disclosure can effectively improve the color cast while achieving multi-angle display. Although Compound C3 is structurally similar to the compounds of the present disclosure, Compound C3 has a complex fused ring group substituted on the arylamine, resulting in the enhancement of molecular planarity and an increase in the refractive index; in spite of the presence of spirofluorenyl that is stereospecific and bulky in Compound C2, Compound C2 is linked to conjugate aryl that is more planar, resulting in an increase in the conjugated area of the entire molecule; F and other groups are not introduced into the molecule of Compound C1 relative to C3, resulting in the enhancement of the molecule refractive index; Br or Cl halogen units are introduced into Compound C4 and Compound C5, changing the molecule polarity and slightly reducing the refractive index.

For a better understanding of the present disclosure, application examples of the compounds of the present disclosure are listed below. It is to be understood by those skilled in the art that the examples described below are used for a better understanding of the present disclosure and are not to be construed as specific limitations to the present disclosure.

### APPLICATION EXAMPLE 1

This application example provides an organic electroluminescent device that was prepared by the specific steps:

1) A glass substrate having an indium tin oxide (ITO) anode layer 2 (with a thickness of 15 nm) was cut into a size of 50 mm×50 mm×0.7 mm, sonicated in isopropyl alcohol and deionized water for 30 minutes separately, and cleaned under ozone for 10 minutes. The cleaned substrate 1 was installed onto a vacuum deposition device.

2) A hole injection layer material Compound a and a p-doped material Compound b were deposited by means of vacuum evaporation at a doping ratio of 3% (mass ratio) on the ITO anode layer 2 as a hole injection layer 3 with a thickness of 5 nm.

3) A hole transport layer material Compound b was deposited by means of vacuum evaporation on the hole injection layer 3 as a first hole transport layer 4 with a thickness of 100 nm.

4) A hole transport material Compound c was deposited by means of vacuum evaporation on the first hole transport layer 4 as a second hole transport layer 5 with a thickness of 5 nm.

5) One layer of emissive layer 6 was deposited by means of vacuum evaporation on the second hole transport layer 5, with a thickness of 5 nm, where Compound d serving as a host material was doped with Compound e serving as a doped material at a ratio of 3% (mass ratio).

6) An electron transport material Compound f was deposited by means of vacuum evaporation on the emissive layer 6 as a first electron transport layer 7 with a thickness of 30 nm.

7) An electron transport material Compound g and an n-doped material Compound h were co-deposited by means of vacuum evaporation at a doping mass ratio of 1:1 on the first electron transport layer 7 as a second electron transport layer 8 with a thickness of 5 nm.

8) A magnesium-silver electrode was deposited by means of vacuum evaporation at a ratio of Mg:Ag of 9:1 on the second electron transport layer 8 as a cathode 9 with a thickness of 10 nm.

9) Compound i was deposited by means of vacuum evaporation on the cathode 9 as a first capping layer 10 with a thickness of 100 nm.

10) The low-refractive-index organic small molecule compound 1 prepared in the present disclosure was deposited by means of vacuum evaporation on the first capping layer 10 as a second capping layer 11 with a thickness of 20 nm.

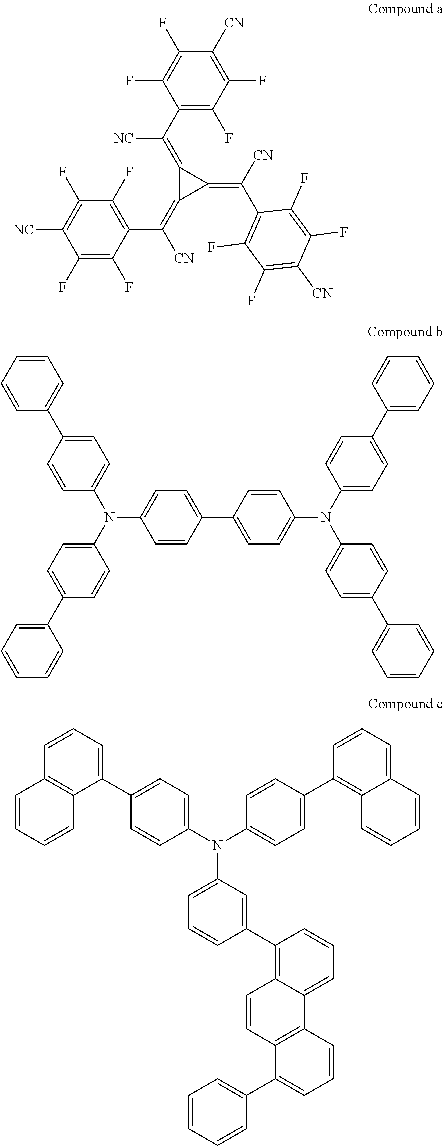 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2cccc3c2ccc2c(-c4cccc(N(c5ccc(-c6cccc7ccccc67)cc5)c5ccc(-c6cccc7ccccc67)cc5)c4)cccc23)cc1]

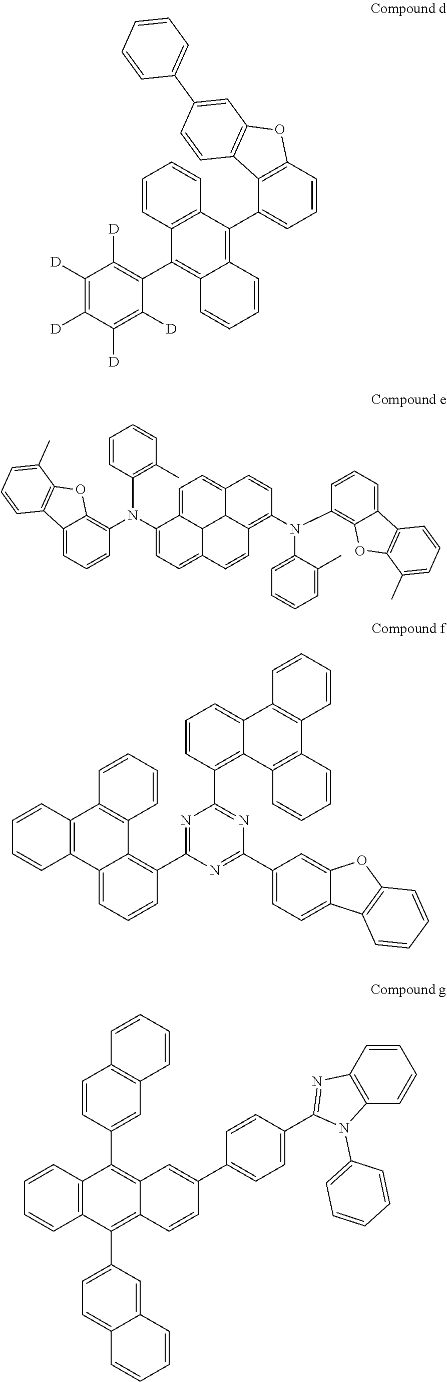

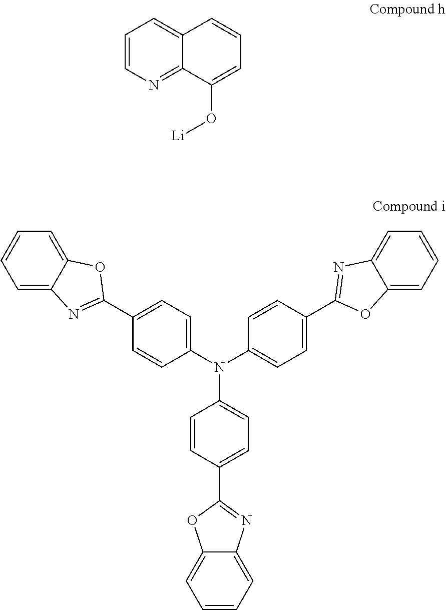

### Device Performance Test

The performance test was performed on the organic electroluminescent devices provided in the application examples and comparative application examples described above.

The currents of each organic electroluminescent device at different voltages were tested using a Keithley 2365A digital nanovoltmeter, and then the obtained currents were divided by the luminescence area to obtain the current densities of each organic electroluminescent device at different voltages. The brightness and radiation energy flux density of each of the organic electroluminescent devices manufactured according to application examples and comparative application examples at different voltages were tested using Konicaminolta CS-2000 spectrometer. According to the current densities and brightness of each organic electroluminescent device at different voltages, a working voltage Vₒₙ (V), current efficiency CE (cd/A), a color cast JNCD (30/45/60° C.and a lifetime LT95 (which is obtained by measuring time taken for the organic electroluminescent device to reach 95% of initial brightness (under a condition of 50 mA/cm²)) at the same current density (10 mA/cm²) were obtained. The results are shown in Table 3.

TABLE 3

| | | | CE_{(10 mA/cm}_{²}₎ | | Lifetime |
|---|---|---|---|---|---|
| No. | Compound | Vₒₙ (V) | (cd A⁻¹) | JNCD | LT95(h) |
| Application | 1 | 3.50 | 7.88 | 4/2/1 | 83 |
| Example 1 | | | | | |
| Application | 2 | 3.51 | 8.21 | 4/3/3 | 85 |
| Example 2 | | | | | |
| Application | 3 | 3.52 | 7.99 | 3/2/1 | 86 |
| Example 3 | | | | | |
| Application | 4 | 3.52 | 8.23 | 5/2/1 | 85 |
| Example 4 | | | | | |
| Application | 7 | 3.50 | 8.00 | 2/2/1 | 86 |
| Example 5 | | | | | |
| Application | 10 | 3.52 | 8.18 | 2/3/1 | 84 |
| Example 6 | | | | | |
| Application | 11 | 3.50 | 8.17 | 3/4/1 | 85 |
| Example 7 | | | | | |
| Application | 15 | 3.49 | 7.88 | 3/3/1 | 86 |
| Example 8 | | | | | |
| Application | 33 | 3.50 | 8.19 | 3/2/1 | 85 |
| Example 9 | | | | | |
| Application | 41 | 3.52 | 8.30 | 2/1/2 | 86 |
| Example 10 | | | | | |
| Application | 109 | 3.52 | 8.12 | 5/4/2 | 84 |
| Example 11 | | | | | |
| Application | 230 | 3.50 | 8.11 | 4/1/3 | 85 |
| Example 12 | | | | | |
| Application | 273 | 3.51 | 8.09 | 3/3/1 | 83 |
| Example 13 | | | | | |
| Application | 285 | 3.49 | 8.11 | 3/3/1 | 85 |
| Example 14 | | | | | |
| Application | 295 | 3.49 | 8.10 | 3/3/2 | 86 |
| Example 15 | | | | | |
| Comparative | C1 | 3.51 | 7.02 | 6/3/3 | 87 |
| Application | | | | | |
| Example 1 | | | | | |
| Comparative | C2 | 3.51 | 6.85 | 5/8/7 | 86 |
| Application | | | | | |
| Example 2 | | | | | |
| Comparative | C3 | 3.50 | 6.88 | 6/4/2 | 85 |
| Application | | | | | |
| Example 3 | | | | | |
| Comparative | C4 | 3.50 | 7.09 | 5/3/4 | 53 |
| Application | | | | | |
| Example 4 | | | | | |
| Comparative | C5 | 3.51 | 7.23 | 6/3/2 | 39 |
| Application | | | | | |
| Example 5 | | | | | |

As can be seen from Table 3, compared with the use of Compound C1, C2 or C3 with the second capping layer material that contains low-refractive-index organic small molecules, the use of the compound provided by the present disclosure as the second capping layer material with the first capping layer material that contains high-refractive-index organic small molecules has a basically equivalent voltage and is more conducive to improving device efficiency, especially significantly improving color cast; although the refractive index becomes small and the device efficiency is improved when C4 and C5 are used, the introduction of Br or Cl greatly reduces device lifetime.

The applicant has stated that although the organic compound and the use thereof in the present disclosure are described through the preceding embodiments, the present disclosure is not limited to the preceding embodiments, which means that the implementation of the present disclosure does not necessarily depend on the preceding embodiments. It is to be apparent to those skilled in the art that any improvements made to the present disclosure, equivalent replacements of raw materials of the product, additions of adjuvant ingredients, selections of specific methods, etc. in the present disclosure all fall within the protection scope and the disclosure scope of the present disclosure.

What is claimed is:

1. An organic compound, wherein the organic compound has a structure represented by Formula I: 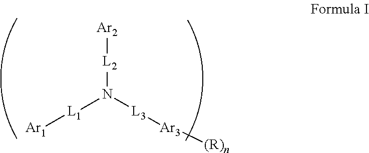
wherein L₁ to L₃ are independently selected from a single bond, C6-C18 arylene or C5-C18 heteroarylene;
Ar₁ to Ar₃ are each independently selected from one of substituted or unsubstituted C6-C30 aryl or substituted or unsubstituted C3-C30 heteroaryl, and at least one of Ar₁ or Ar₃ is selected from any one of the following groups 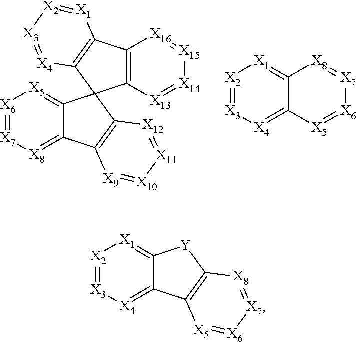
wherein a site of attachment of the group is any attachable position;
X₁ to X₁₆ are independently C—R₁ or N, and R₁ is hydrogen, C6-C18 aryl or C5-C18 heteroaryl; Y is O, S, N or Si;
R is F, —CHF₂, CH₂F, —CF₃ or —CN, wherein n is the number of R and is an integer greater than or equal to 1.

2. The organic compound according to claim 1, wherein L₁ to L₃ are independently selected from any one of a single bond, substituted or unsubstituted phenyl, substituted or unsubstituted biphenyl, substituted or unsubstituted naphthyl, substituted or unsubstituted phenanthryl, substituted or unsubstituted triphenylene, substituted or unsubstituted fluorenyl, substituted or unsubstituted carbazolyl, substituted or unsubstituted dibenzofuryl, substituted or unsubstituted dibenzothienyl, substituted or unsubstituted pyridyl, substituted or unsubstituted pyrimidinyl, or substituted or unsubstituted triazinyl; a substituent in the substituted group is selected from fluorine, trifluoromethyl, cyano, methyl, ethyl, t-butyl, isopropyl or methoxy.

3. The organic compound according to claim 1, wherein Ar₁ to Ar₃ are independently selected from any one of phenyl, biphenyl, naphthyl, 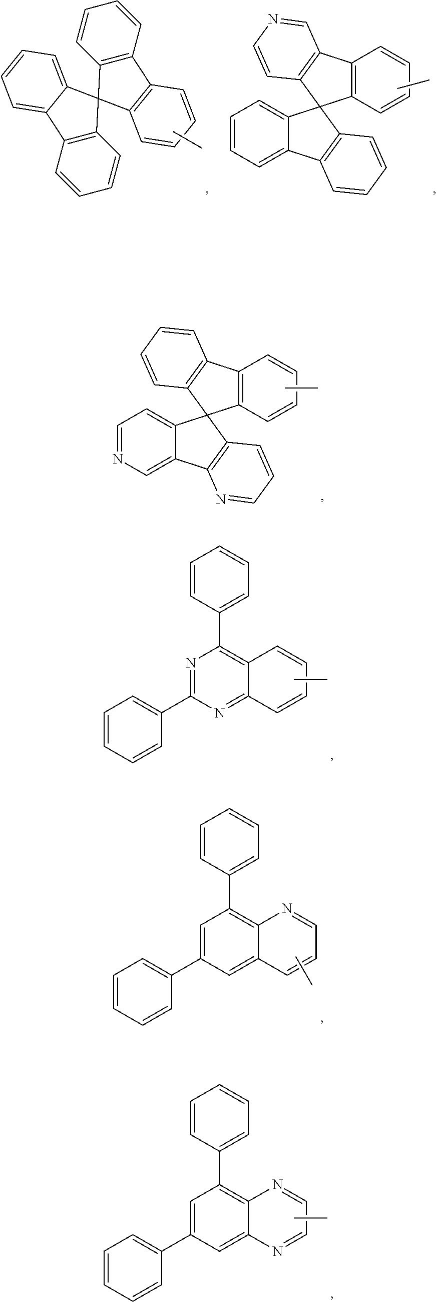 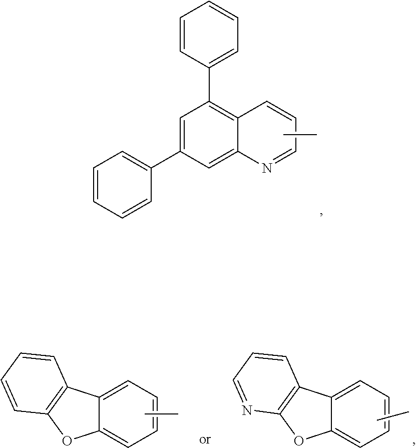
and at least one of Ar₁ to Ar₃ is selected from any one of naphthyl,

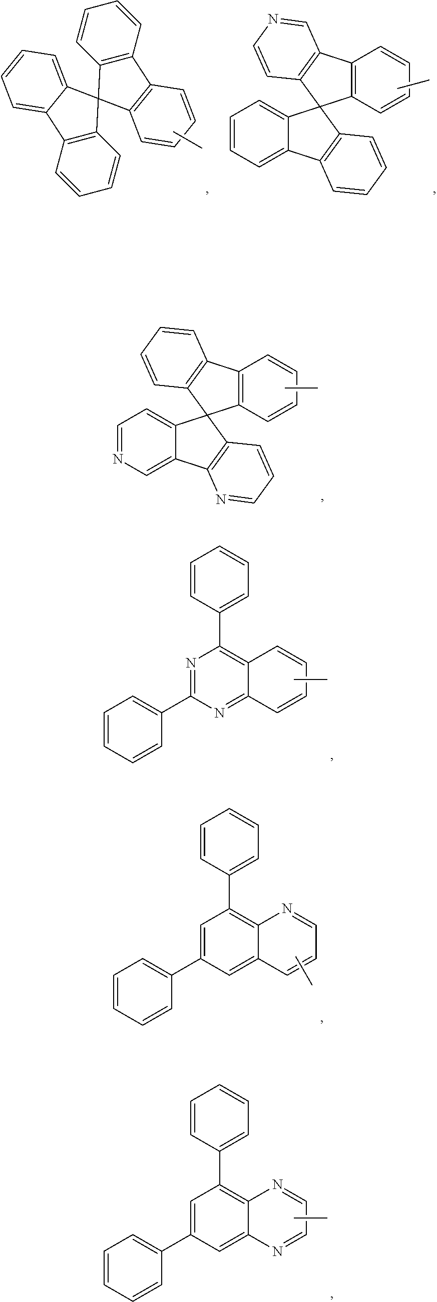

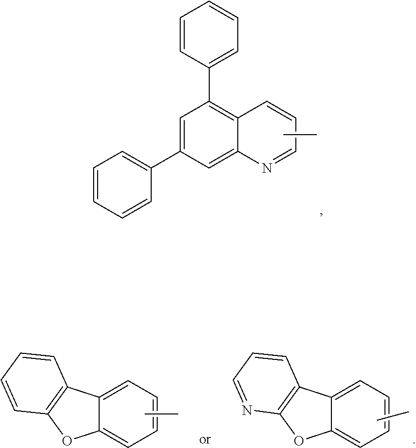

4. The organic compound according to claim 1, wherein n is an integer of 1 to 3.

5. The organic compound according to claim 1, wherein the organic compound is any one of the following compounds:

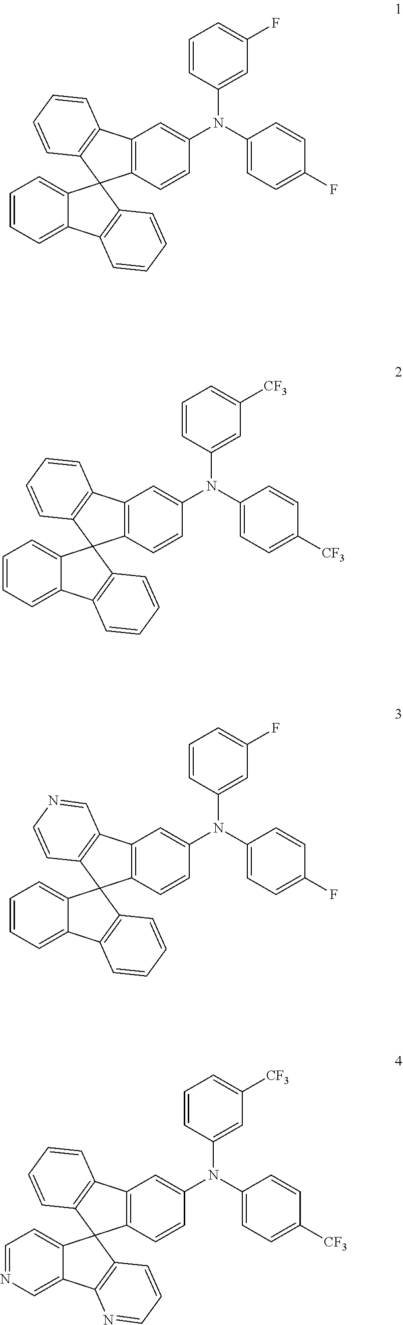 [SMILES: Fc1ccc(N(c2cccc(F)c2)c2ccc3c(c2)-c2ccccc2C32c3ccccc3-c3ccccc32)cc1 ; FC(F)(F)c1ccc(N(c2cccc(C(F)(F)F)c2)c2ccc3c(c2)-c2ccccc2C32c3ccccc3-c3ccccc32)cc1 ; Fc1ccc(N(c2cccc(F)c2)c2ccc3c(c2)-c2cnccc2C32c3ccccc3-c3ccccc32)cc1 ; FC(F)(F)c1ccc(N(c2cccc(C(F)(F)F)c2)c2ccc3c(c2)-c2ccccc2C32c3ccncc3-c3ncccc32)cc1]

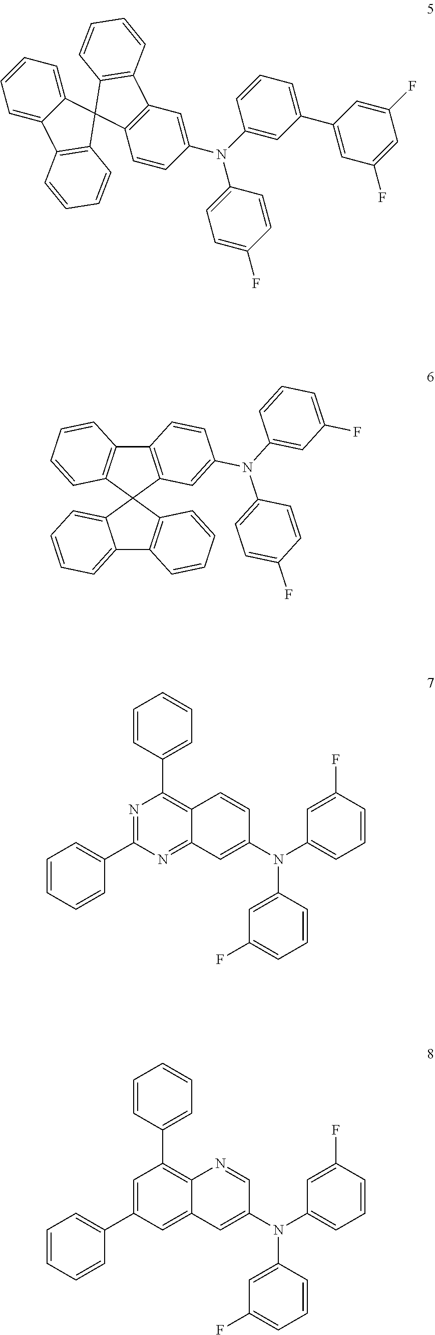 [SMILES: Fc1ccc(N(c2cccc(-c3cc(F)cc(F)c3)c2)c2ccc3c(c2)-c2ccccc2C32c3ccccc3-c3ccccc32)cc1 ; Fc1ccc(N(c2cccc(F)c2)c2ccc3c(c2)C2(c4ccccc4-c4ccccc42)c2ccccc2-3)cc1 ; Fc1cccc(N(c2cccc(F)c2)c2ccc3c(-c4ccccc4)nc(-c4ccccc4)nc3c2)c1 ; Fc1cccc(N(c2cccc(F)c2)c2cnc3c(-c4ccccc4)cc(-c4ccccc4)cc3c2)c1]

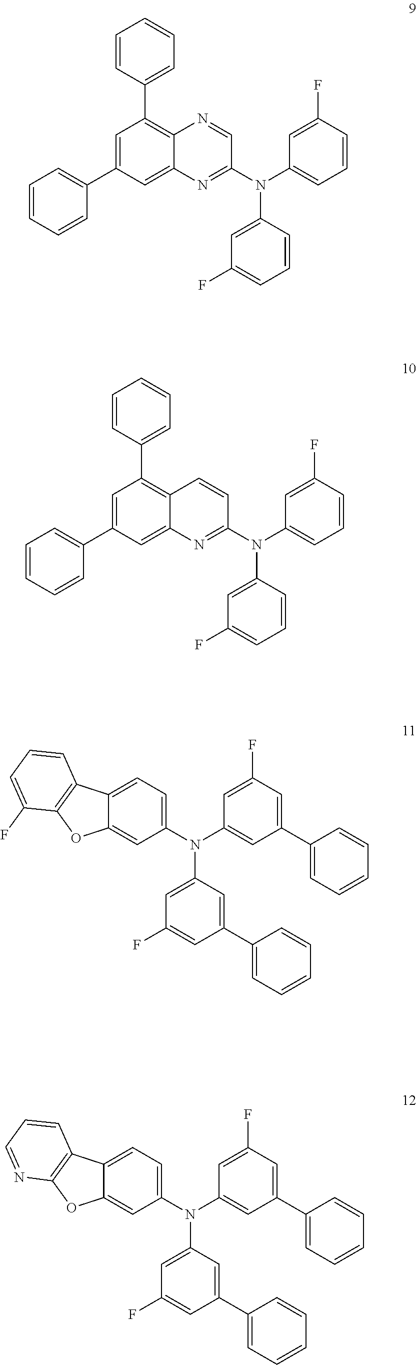 [SMILES: Fc1cccc(N(c2cccc(F)c2)c2cnc3c(-c4ccccc4)cc(-c4ccccc4)cc3n2)c1 ; Fc1cccc(N(c2cccc(F)c2)c2ccc3c(-c4ccccc4)cc(-c4ccccc4)cc3n2)c1 ; Fc1cc(-c2ccccc2)cc(N(c2cc(F)cc(-c3ccccc3)c2)c2ccc3c(c2)oc2c(F)cccc23)c1 ; Fc1cc(-c2ccccc2)cc(N(c2cc(F)cc(-c3ccccc3)c2)c2ccc3c(c2)oc2ncccc23)c1]

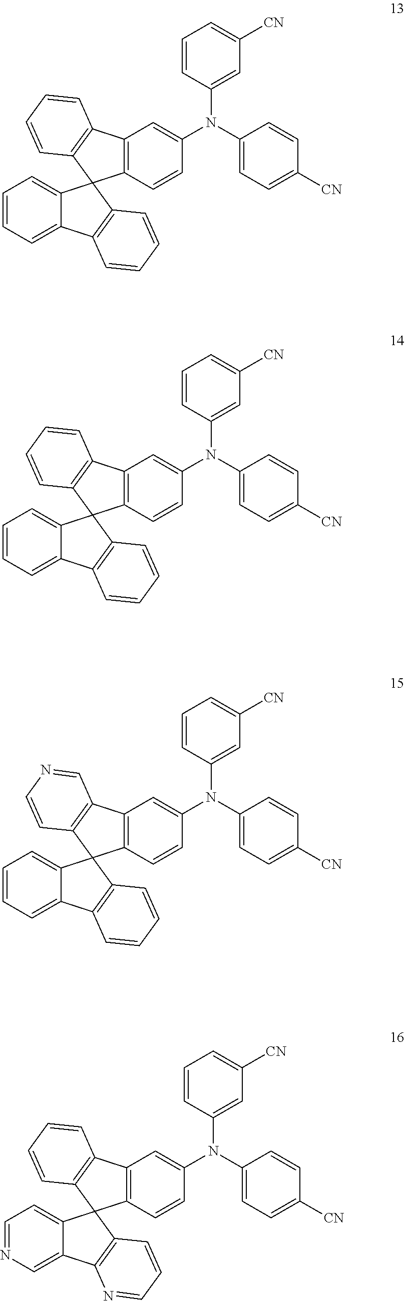 [SMILES: N#Cc1ccc(N(c2cccc(C#N)c2)c2ccc3c(c2)-c2ccccc2C32c3ccccc3-c3ccccc32)cc1 ; N#Cc1ccc(N(c2cccc(C#N)c2)c2ccc3c(c2)-c2ccccc2C32c3ccccc3-c3ccccc32)cc1 ; N#Cc1ccc(N(c2cccc(C#N)c2)c2ccc3c(c2)-c2cnccc2C32c3ccccc3-c3ccccc32)cc1 ; N#Cc1ccc(N(c2cccc(C#N)c2)c2ccc3c(c2)-c2ccccc2C32c3ccncc3-c3ncccc32)cc1]

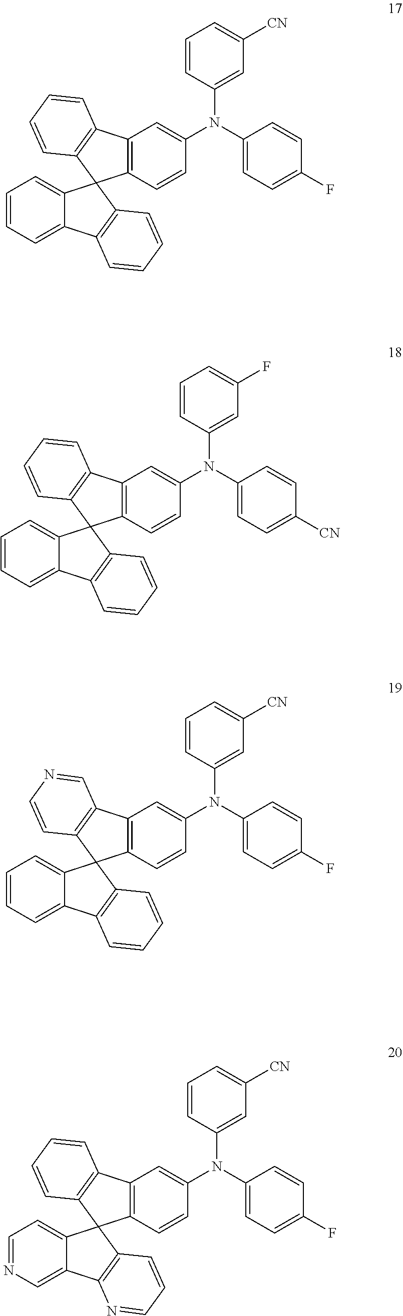 [SMILES: N#Cc1cccc(N(c2ccc(F)cc2)c2ccc3c(c2)-c2ccccc2C32c3ccccc3-c3ccccc32)c1 ; N#Cc1ccc(N(c2cccc(F)c2)c2ccc3c(c2)-c2ccccc2C32c3ccccc3-c3ccccc32)cc1 ; N#Cc1cccc(N(c2ccc(F)cc2)c2ccc3c(c2)-c2cnccc2C32c3ccccc3-c3ccccc32)c1 ; N#Cc1cccc(N(c2ccc(F)cc2)c2ccc3c(c2)-c2ccccc2C32c3ccncc3-c3ncccc32)c1]

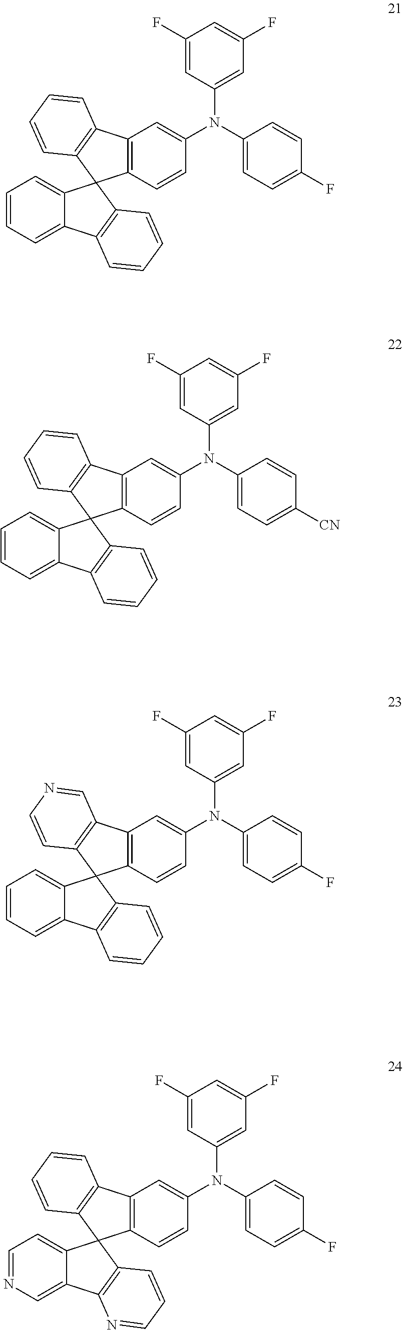 [SMILES: Fc1ccc(N(c2cc(F)cc(F)c2)c2ccc3c(c2)-c2ccccc2C32c3ccccc3-c3ccccc32)cc1 ; N#Cc1ccc(N(c2cc(F)cc(F)c2)c2ccc3c(c2)-c2ccccc2C32c3ccccc3-c3ccccc32)cc1 ; Fc1ccc(N(c2cc(F)cc(F)c2)c2ccc3c(c2)-c2cnccc2C32c3ccccc3-c3ccccc32)cc1 ; Fc1ccc(N(c2cc(F)cc(F)c2)c2ccc3c(c2)-c2ccccc2C32c3ccncc3-c3ncccc32)cc1]

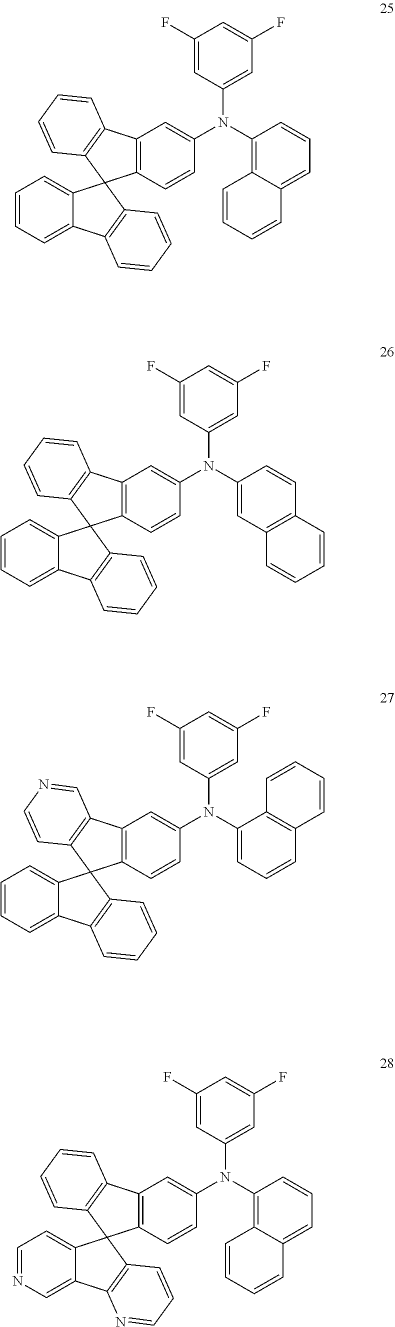 [SMILES: Fc1cc(F)cc(N(c2ccc3c(c2)-c2ccccc2C32c3ccccc3-c3ccccc32)c2cccc3ccccc23)c1 ; Fc1cc(F)cc(N(c2ccc3c(c2)-c2ccccc2C32c3ccccc3-c3ccccc32)c2ccc3ccccc3c2)c1 ; Fc1cc(F)cc(N(c2ccc3c(c2)-c2cnccc2C32c3ccccc3-c3ccccc32)c2cccc3ccccc23)c1 ; Fc1cc(F)cc(N(c2ccc3c(c2)-c2ccccc2C32c3ccncc3-c3ncccc32)c2cccc3ccccc23)c1]

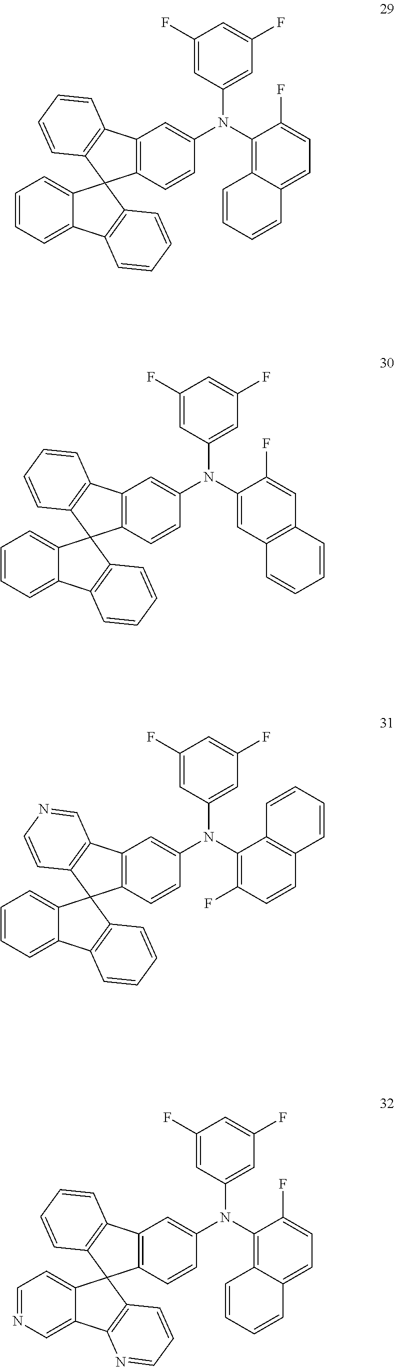 [SMILES: Fc1cc(F)cc(N(c2ccc3c(c2)-c2ccccc2C32c3ccccc3-c3ccccc32)c2c(F)ccc3ccccc23)c1 ; Fc1cc(F)cc(N(c2ccc3c(c2)-c2ccccc2C32c3ccccc3-c3ccccc32)c2cc3ccccc3cc2F)c1 ; Fc1cc(F)cc(N(c2ccc3c(c2)-c2cnccc2C32c3ccccc3-c3ccccc32)c2c(F)ccc3ccccc23)c1 ; Fc1cc(F)cc(N(c2ccc3c(c2)-c2ccccc2C32c3ccncc3-c3ncccc32)c2c(F)ccc3ccccc23)c1]

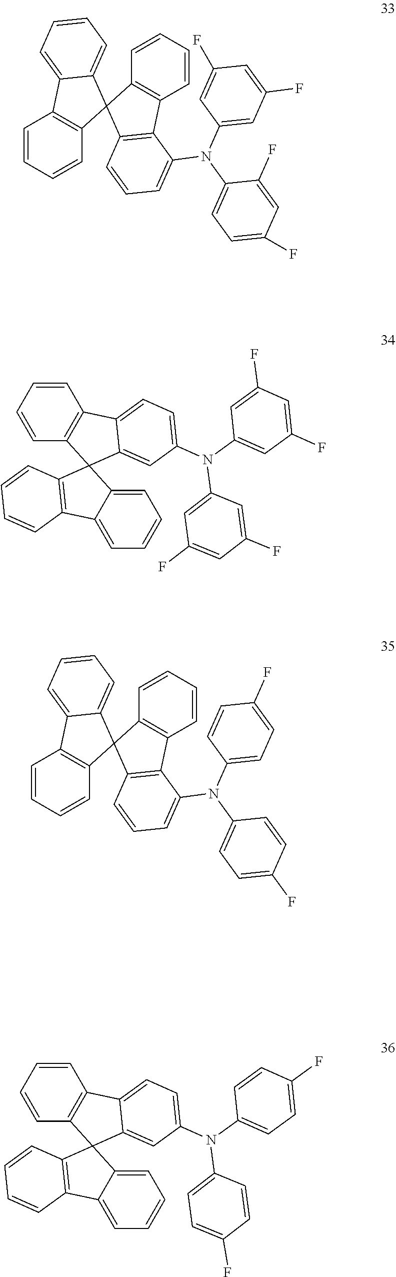 [SMILES: Fc1cc(F)cc(N(c2ccc(F)cc2F)c2cccc3c2-c2ccccc2C32c3ccccc3-c3ccccc32)c1 ; Fc1cc(F)cc(N(c2cc(F)cc(F)c2)c2ccc3c(c2)C2(c4ccccc4-c4ccccc42)c2ccccc2-3)c1 ; Fc1ccc(N(c2ccc(F)cc2)c2cccc3c2-c2ccccc2C32c3ccccc3-c3ccccc32)cc1 ; Fc1ccc(N(c2ccc(F)cc2)c2ccc3c(c2)C2(c4ccccc4-c4ccccc42)c2ccccc2-3)cc1]

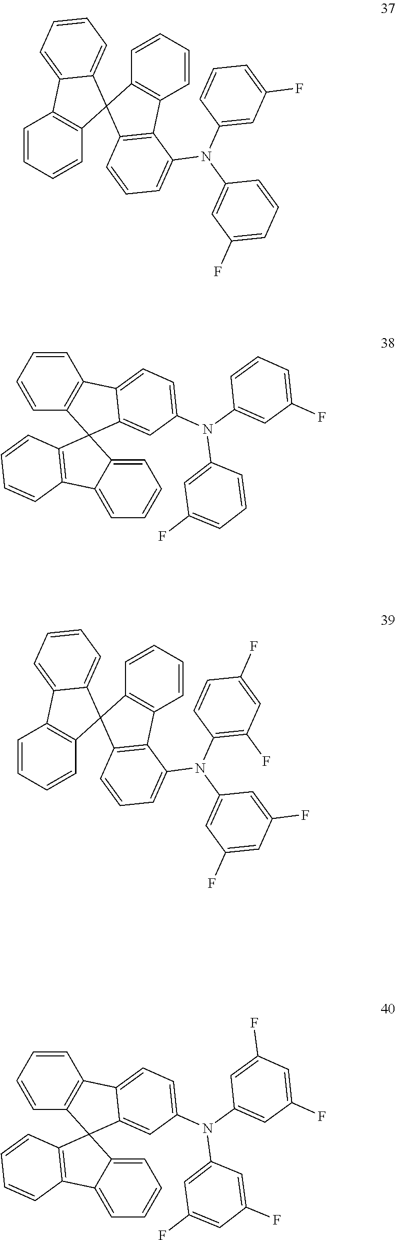 [SMILES: Fc1cccc(N(c2cccc(F)c2)c2cccc3c2-c2ccccc2C32c3ccccc3-c3ccccc32)c1 ; Fc1cccc(N(c2cccc(F)c2)c2ccc3c(c2)C2(c4ccccc4-c4ccccc42)c2ccccc2-3)c1 ; Fc1cc(F)cc(N(c2ccc(F)cc2F)c2cccc3c2-c2ccccc2C32c3ccccc3-c3ccccc32)c1 ; Fc1cc(F)cc(N(c2cc(F)cc(F)c2)c2ccc3c(c2)C2(c4ccccc4-c4ccccc42)c2ccccc2-3)c1]

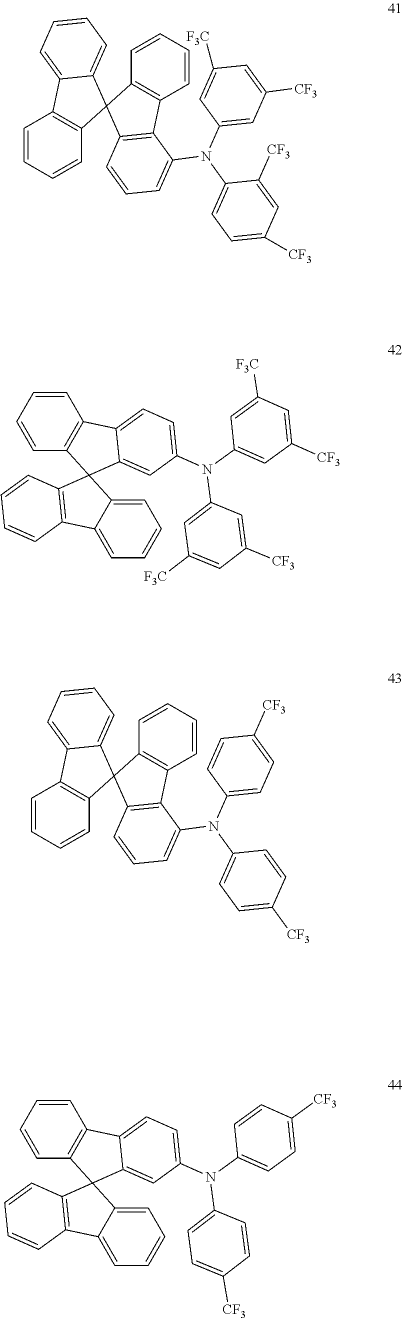 [SMILES: FC(F)(F)c1cc(N(c2ccc(C(F)(F)F)cc2C(F)(F)F)c2cccc3c2-c2ccccc2C32c3ccccc3-c3ccccc32)cc(C(F)(F)F)c1 ; FC(F)(F)c1cc(N(c2cc(C(F)(F)F)cc(C(F)(F)F)c2)c2ccc3c(c2)C2(c4ccccc4-c4ccccc42)c2ccccc2-3)cc(C(F)(F)F)c1 ; FC(F)(F)c1ccc(N(c2ccc(C(F)(F)F)cc2)c2cccc3c2-c2ccccc2C32c3ccccc3-c3ccccc32)cc1 ; FC(F)(F)c1ccc(N(c2ccc(C(F)(F)F)cc2)c2ccc3c(c2)C2(c4ccccc4-c4ccccc42)c2ccccc2-3)cc1]

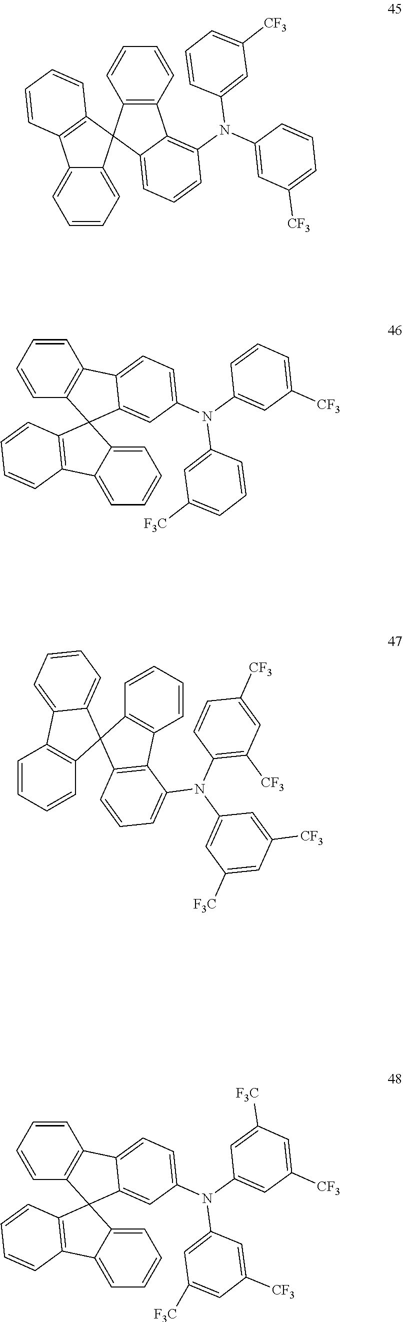 [SMILES: FC(F)(F)c1cccc(N(c2cccc(C(F)(F)F)c2)c2cccc3c2-c2ccccc2C32c3ccccc3-c3ccccc32)c1 ; FC(F)(F)c1cccc(N(c2cccc(C(F)(F)F)c2)c2ccc3c(c2)C2(c4ccccc4-c4ccccc42)c2ccccc2-3)c1 ; FC(F)(F)c1cc(N(c2ccc(C(F)(F)F)cc2C(F)(F)F)c2cccc3c2-c2ccccc2C32c3ccccc3-c3ccccc32)cc(C(F)(F)F)c1 ; FC(F)(F)c1cc(N(c2cc(C(F)(F)F)cc(C(F)(F)F)c2)c2ccc3c(c2)C2(c4ccccc4-c4ccccc42)c2ccccc2-3)cc(C(F)(F)F)c1]

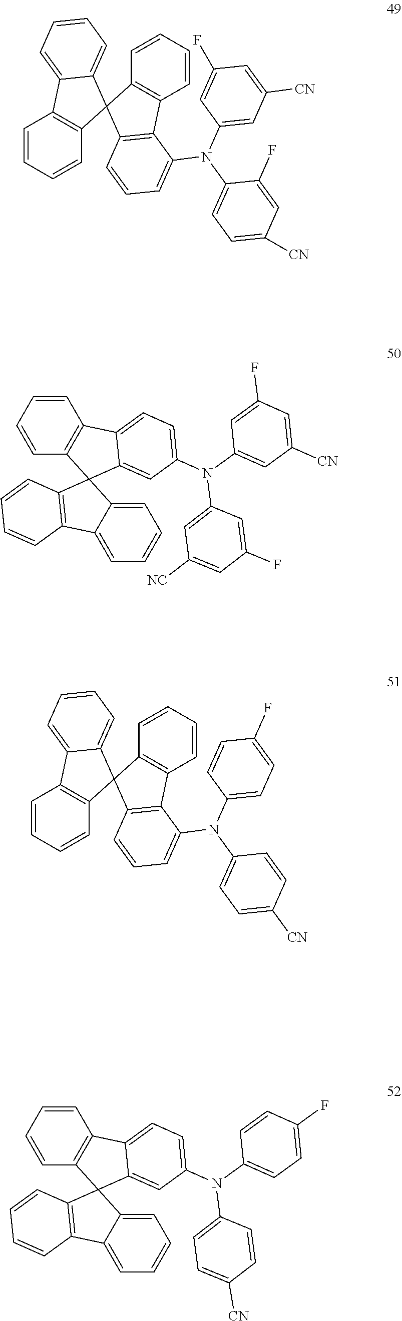 [SMILES: N#Cc1cc(F)cc(N(c2ccc(C#N)cc2F)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1 ; N#Cc1ccc(N(c2ccc(F)cc2)c2cccc3c2-c2ccccc2C32c3ccccc3-c3ccccc32)cc1 ; N#Cc1ccc(N(c2ccc(F)cc2)c2ccc3c(c2)C2(c4ccccc4-c4ccccc42)c2ccccc2-3)cc1]

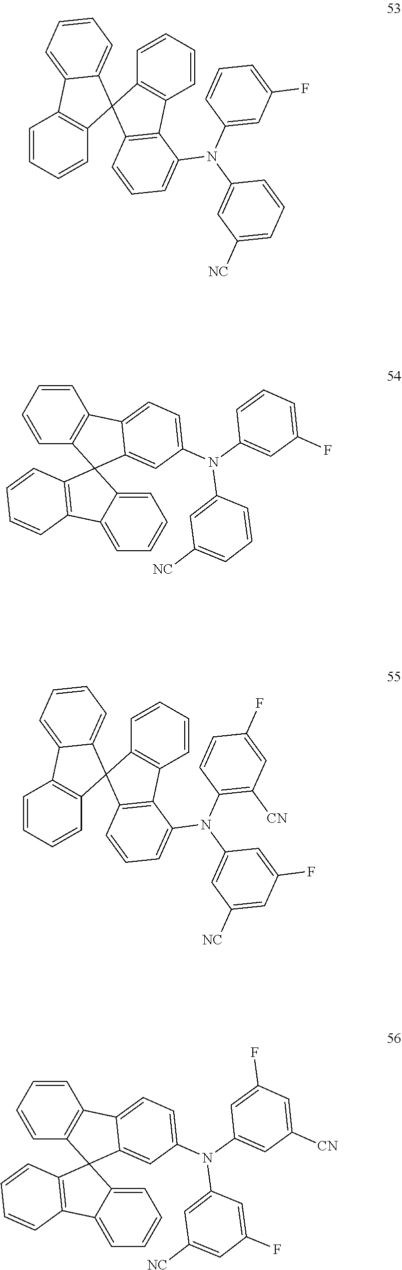 [SMILES: N#Cc1cccc(N(c2cccc(F)c2)c2cccc3c2-c2ccccc2C32c3ccccc3-c3ccccc32)c1 ; N#Cc1cccc(N(c2cccc(F)c2)c2ccc3c(c2)C2(c4ccccc4-c4ccccc42)c2ccccc2-3)c1 ; N#Cc1cc(F)cc(N(c2ccc(F)cc2C#N)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1]

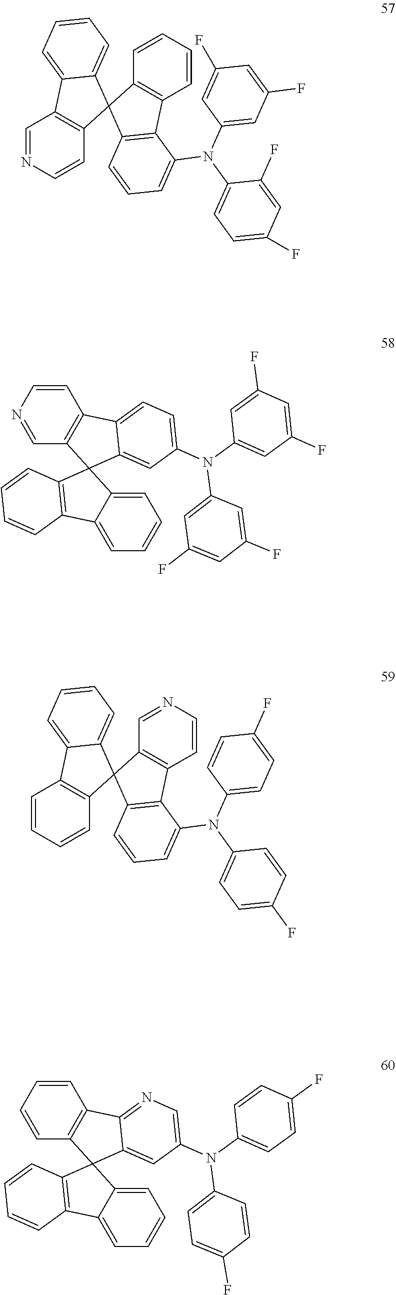 [SMILES: Fc1cc(F)cc(N(c2ccc(F)cc2F)c2cccc3c2-c2ccccc2C32c3ccccc3-c3cnccc32)c1 ; Fc1cc(F)cc(N(c2cc(F)cc(F)c2)c2ccc3c(c2)C2(c4ccccc4-c4ccccc42)c2cnccc2-3)c1 ; Fc1ccc(N(c2ccc(F)cc2)c2cccc3c2-c2ccncc2C32c3ccccc3-c3ccccc32)cc1 ; Fc1ccc(N(c2ccc(F)cc2)c2cnc3c(c2)C2(c4ccccc4-c4ccccc42)c2ccccc2-3)cc1]

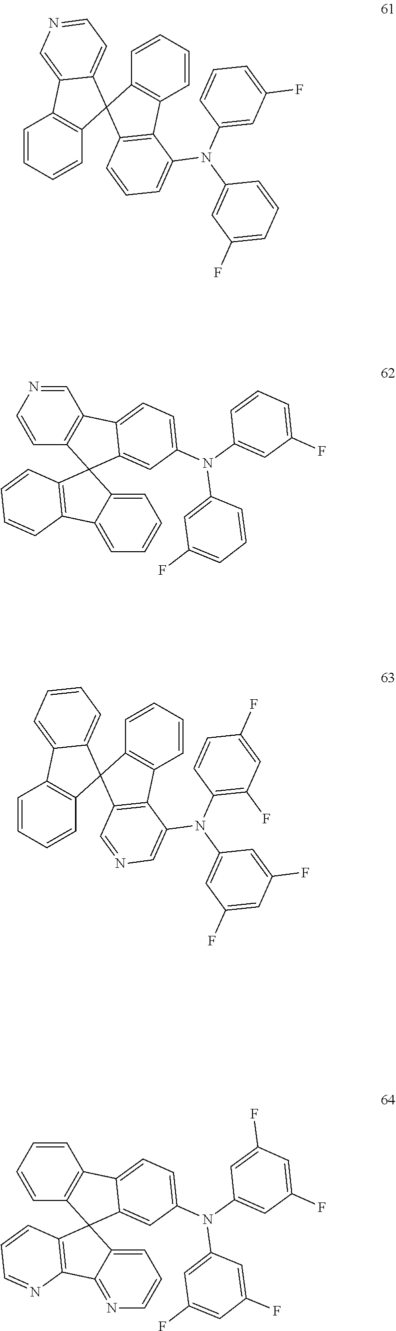 [SMILES: Fc1cccc(N(c2cccc(F)c2)c2cccc3c2-c2ccccc2C32c3ccccc3-c3cnccc32)c1 ; Fc1cccc(N(c2cccc(F)c2)c2ccc3c(c2)C2(c4ccccc4-c4ccccc42)c2ccncc2-3)c1 ; Fc1cc(F)cc(N(c2ccc(F)cc2F)c2cncc3c2-c2ccccc2C32c3ccccc3-c3ccccc32)c1 ; Fc1cc(F)cc(N(c2cc(F)cc(F)c2)c2ccc3c(c2)C2(c4ccccc4-3)c3cccnc3-c3ncccc32)c1]

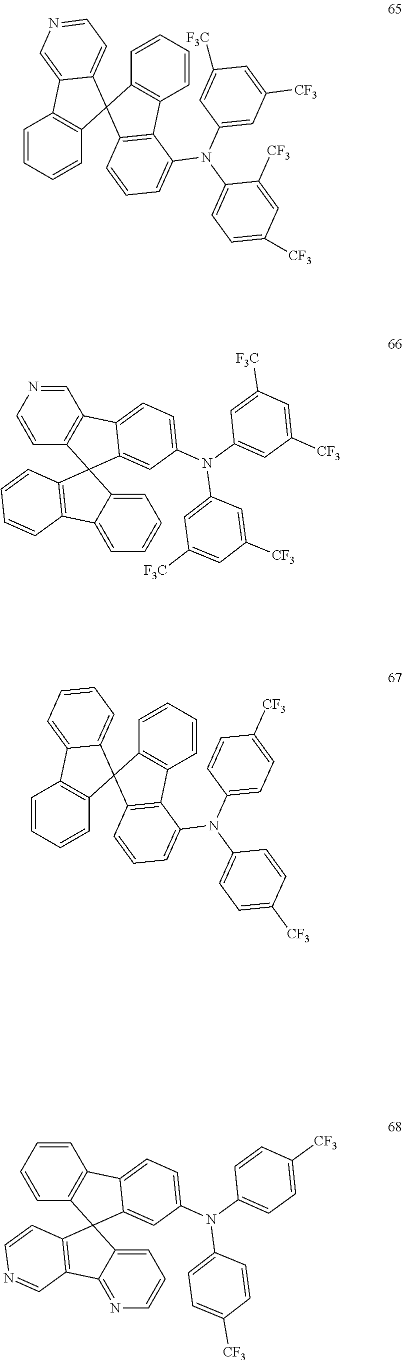 [SMILES: FC(F)(F)c1cc(N(c2ccc(C(F)(F)F)cc2C(F)(F)F)c2cccc3c2-c2ccccc2C32c3ccccc3-c3cnccc32)cc(C(F)(F)F)c1 ; FC(F)(F)c1cc(N(c2cc(C(F)(F)F)cc(C(F)(F)F)c2)c2ccc3c(c2)C2(c4ccccc4-c4ccccc42)c2ccncc2-3)cc(C(F)(F)F)c1 ; FC(F)(F)c1ccc(N(c2ccc(C(F)(F)F)cc2)c2cccc3c2-c2ccccc2C32c3ccccc3-c3ccccc32)cc1 ; FC(F)(F)c1ccc(N(c2ccc(C(F)(F)F)cc2)c2ccc3c(c2)C2(c4ccccc4-3)c3ccncc3-c3ncccc32)cc1]

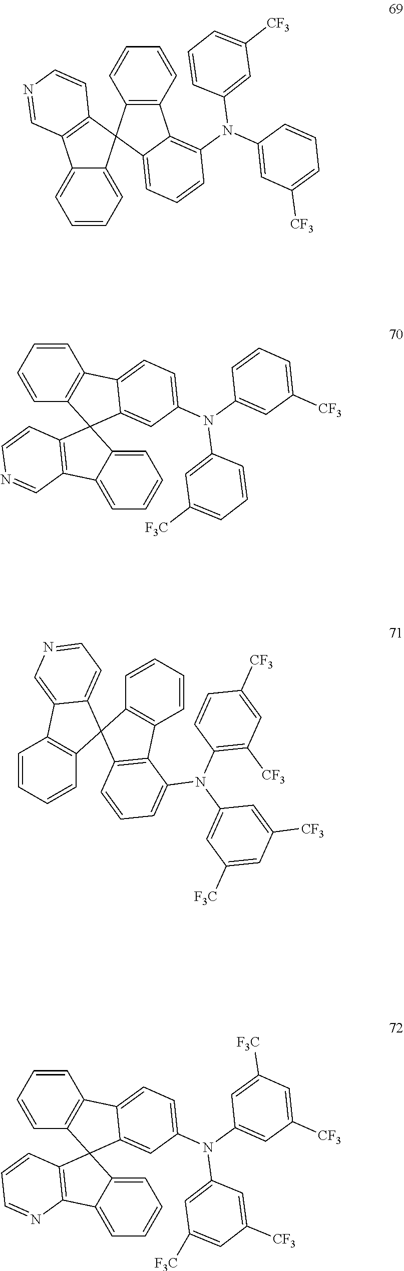 [SMILES: FC(F)(F)c1cccc(N(c2cccc(C(F)(F)F)c2)c2cccc3c2-c2ccccc2C32c3ccccc3-c3cnccc32)c1 ; FC(F)(F)c1cccc(N(c2cccc(C(F)(F)F)c2)c2ccc3c(c2)C2(c4ccccc4-c4cnccc42)c2ccccc2-3)c1 ; FC(F)(F)c1cc(N(c2ccc(C(F)(F)F)cc2C(F)(F)F)c2cccc3c2-c2ccccc2C32c3ccccc3-c3cnccc32)cc(C(F)(F)F)c1 ; FC(F)(F)c1cc(N(c2cc(C(F)(F)F)cc(C(F)(F)F)c2)c2ccc3c(c2)C2(c4ccccc4-3)c3ccccc3-c3ncccc32)cc(C(F)(F)F)c1]

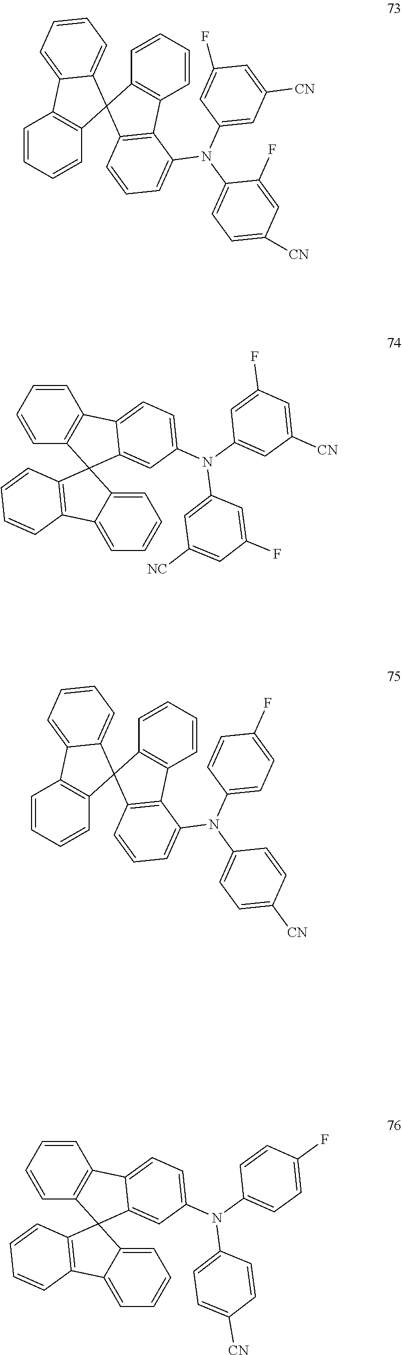 [SMILES: N#Cc1cc(F)cc(N(c2ccc(C#N)cc2F)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1 ; N#Cc1ccc(N(c2ccc(F)cc2)c2cccc3c2-c2ccccc2C32c3ccccc3-c3ccccc32)cc1 ; N#Cc1ccc(N(c2ccc(F)cc2)c2ccc3c(c2)C2(c4ccccc4-c4ccccc42)c2ccccc2-3)cc1]

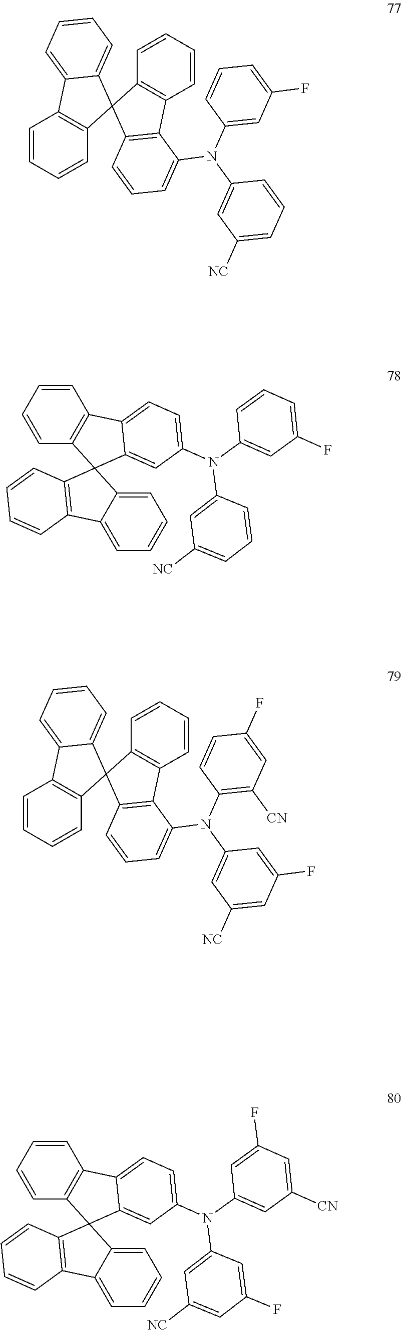 [SMILES: N#Cc1cccc(N(c2cccc(F)c2)c2cccc3c2-c2ccccc2C32c3ccccc3-c3ccccc32)c1 ; N#Cc1cccc(N(c2cccc(F)c2)c2ccc3c(c2)C2(c4ccccc4-c4ccccc42)c2ccccc2-3)c1 ; N#Cc1cc(F)cc(N(c2ccc(F)cc2C#N)c2cccc3c2-c2ccccc2C32c3ccccc3-c3ccccc32)c1 ; N#Cc1cc(F)cc(N(c2cc(F)cc(C#N)c2)c2ccc3c(c2)C2(c4ccccc4-c4ccccc42)c2ccccc2-3)c1]

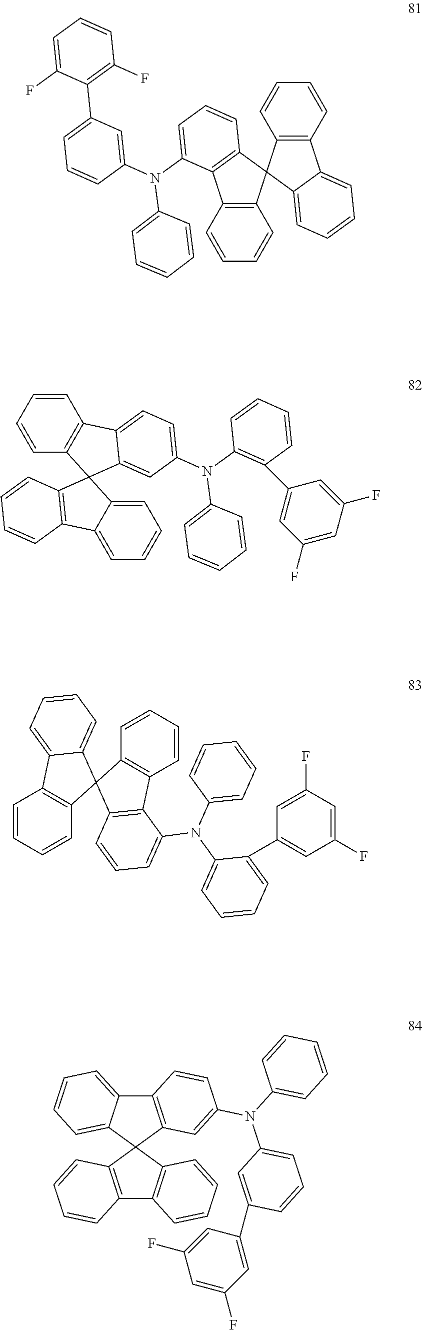 [SMILES: Fc1cccc(F)c1-c1cccc(N(c2ccccc2)c2cccc3c2-c2ccccc2C32c3ccccc3-c3ccccc32)c1 ; Fc1cc(F)cc(-c2ccccc2N(c2ccccc2)c2ccc3c(c2)C2(c4ccccc4-c4ccccc42)c2ccccc2-3)c1 ; Fc1cc(F)cc(-c2ccccc2N(c2ccccc2)c2cccc3c2-c2ccccc2C32c3ccccc3-c3ccccc32)c1 ; Fc1cc(F)cc(-c2cccc(N(c3ccccc3)c3ccc4c(c3)C3(c5ccccc5-c5ccccc53)c3ccccc3-4)c2)c1]

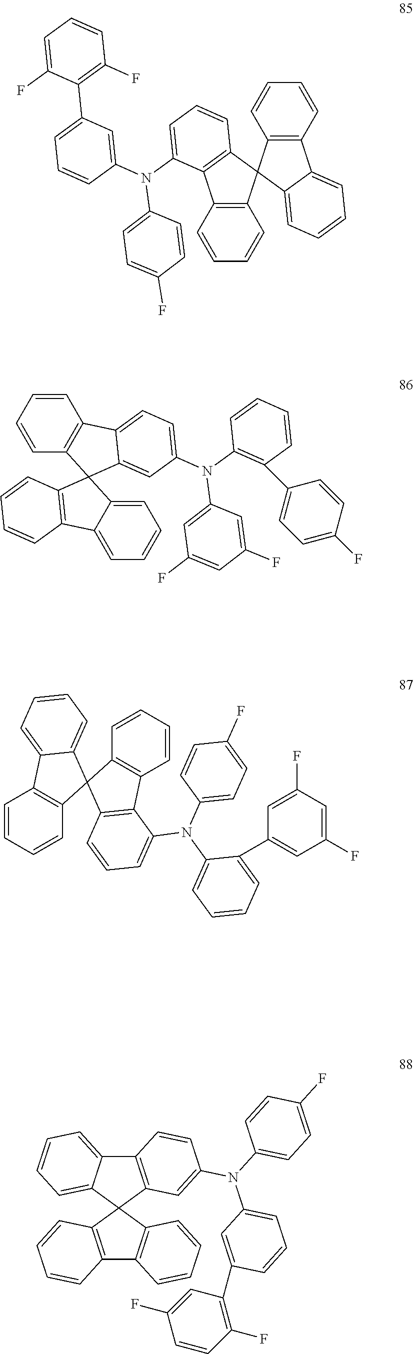 [SMILES: Fc1ccc(N(c2cccc(-c3c(F)cccc3F)c2)c2cccc3c2-c2ccccc2C32c3ccccc3-c3ccccc32)cc1 ; Fc1ccc(-c2ccccc2N(c2cc(F)cc(F)c2)c2ccc3c(c2)C2(c4ccccc4-c4ccccc42)c2ccccc2-3)cc1 ; Fc1ccc(N(c2ccccc2-c2cc(F)cc(F)c2)c2cccc3c2-c2ccccc2C32c3ccccc3-c3ccccc32)cc1 ; Fc1ccc(N(c2cccc(-c3cc(F)ccc3F)c2)c2ccc3c(c2)C2(c4ccccc4-c4ccccc42)c2ccccc2-3)cc1]

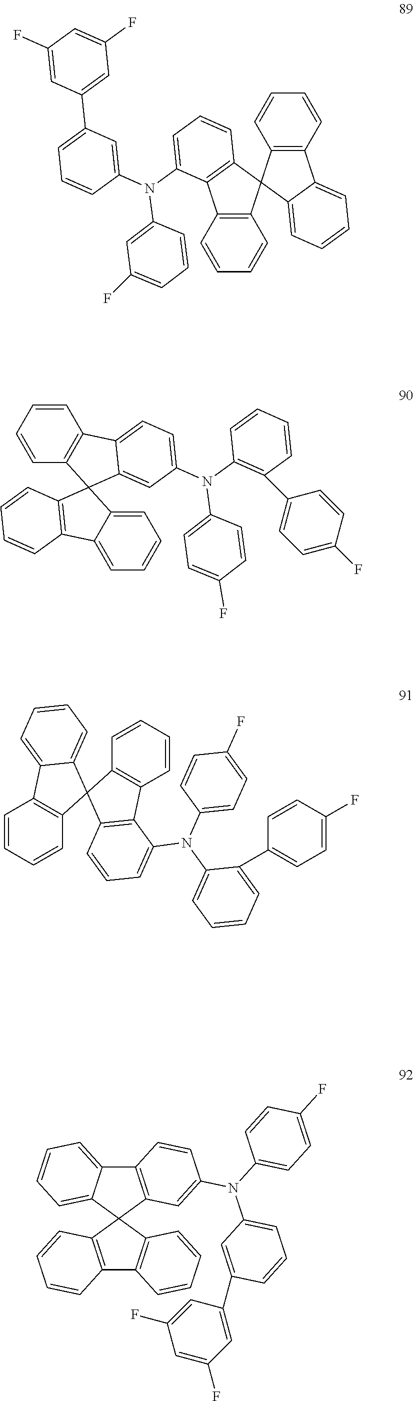 [SMILES: Fc1cc(F)cc(-c2cccc(N(c3cccc(F)c3)c3cccc4c3-c3ccccc3C43c4ccccc4-c4ccccc43)c2)c1 ; Fc1ccc(-c2ccccc2N(c2ccc(F)cc2)c2ccc3c(c2)C2(c4ccccc4-c4ccccc42)c2ccccc2-3)cc1 ; Fc1ccc(-c2ccccc2N(c2ccc(F)cc2)c2cccc3c2-c2ccccc2C32c3ccccc3-c3ccccc32)cc1 ; Fc1ccc(N(c2cccc(-c3cc(F)cc(F)c3)c2)c2ccc3c(c2)C2(c4ccccc4-c4ccccc42)c2ccccc2-3)cc1]

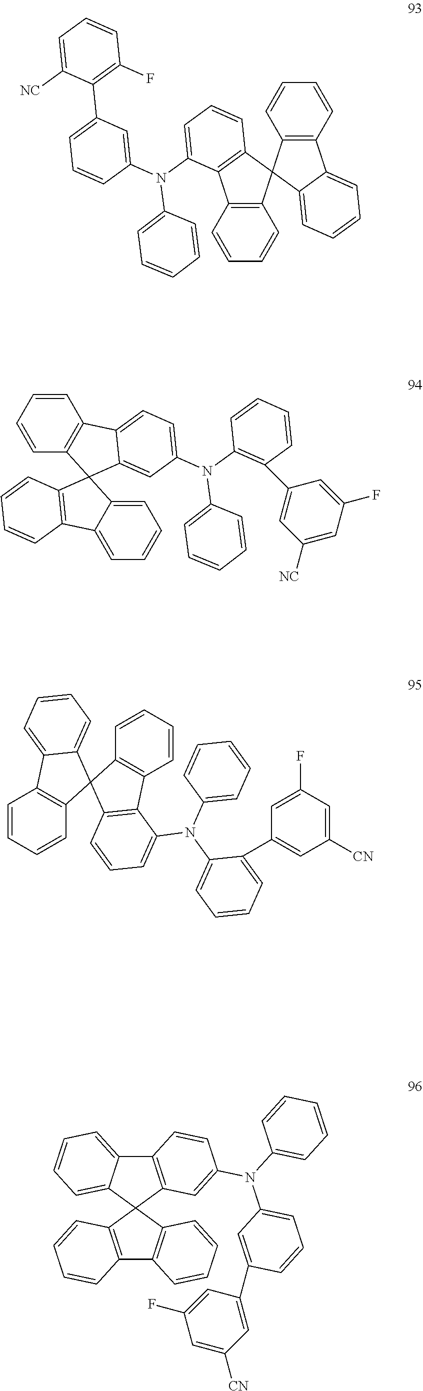 [SMILES: N#Cc1cccc(F)c1-c1cccc(N(c2ccccc2)c2cccc3c2-c2ccccc2C32c3ccccc3-c3ccccc32)c1 ; N#Cc1cc(F)cc(-c2ccccc2N(c2ccccc2)c2ccc3c(c2)C2(c4ccccc4-c4ccccc42)c2ccccc2-3)c1 ; N#Cc1cc(F)cc(-c2ccccc2N(c2ccccc2)c2cccc3c2-c2ccccc2C32c3ccccc3-c3ccccc32)c1 ; N#Cc1cc(F)cc(-c2cccc(N(c3ccccc3)c3ccc4c(c3)C3(c5ccccc5-c5ccccc53)c3ccccc3-4)c2)c1]

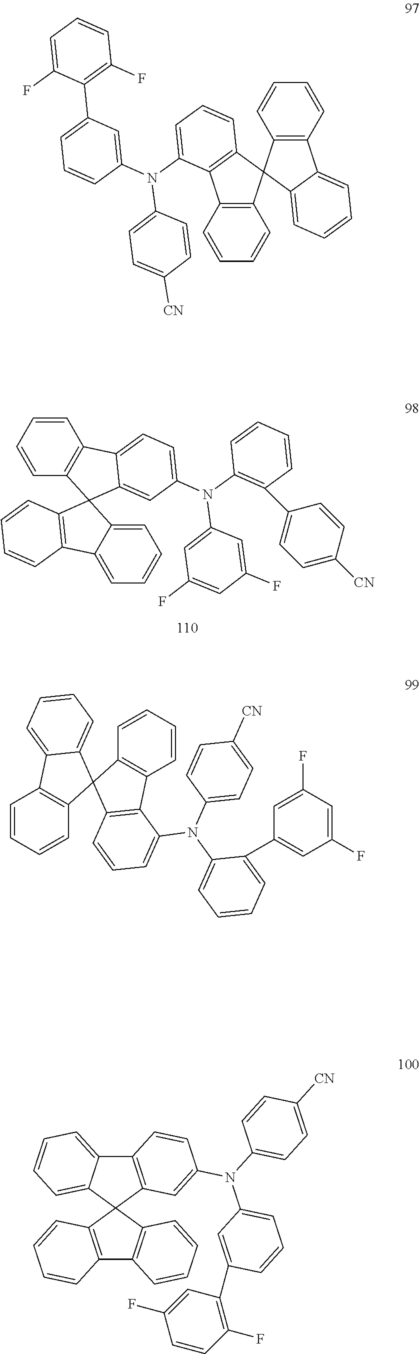 [SMILES: N#Cc1ccc(N(c2cccc(-c3c(F)cccc3F)c2)c2cccc3c2-c2ccccc2C32c3ccccc3-c3ccccc32)cc1 ; N#Cc1ccc(-c2ccccc2N(c2cc(F)cc(F)c2)c2ccc3c(c2)C2(c4ccccc4-c4ccccc42)c2ccccc2-3)cc1 ; N#Cc1ccc(N(c2ccccc2-c2cc(F)cc(F)c2)c2cccc3c2-c2ccccc2C32c3ccccc3-c3ccccc32)cc1 ; N#Cc1ccc(N(c2cccc(-c3cc(F)ccc3F)c2)c2ccc3c(c2)C2(c4ccccc4-c4ccccc42)c2ccccc2-3)cc1]

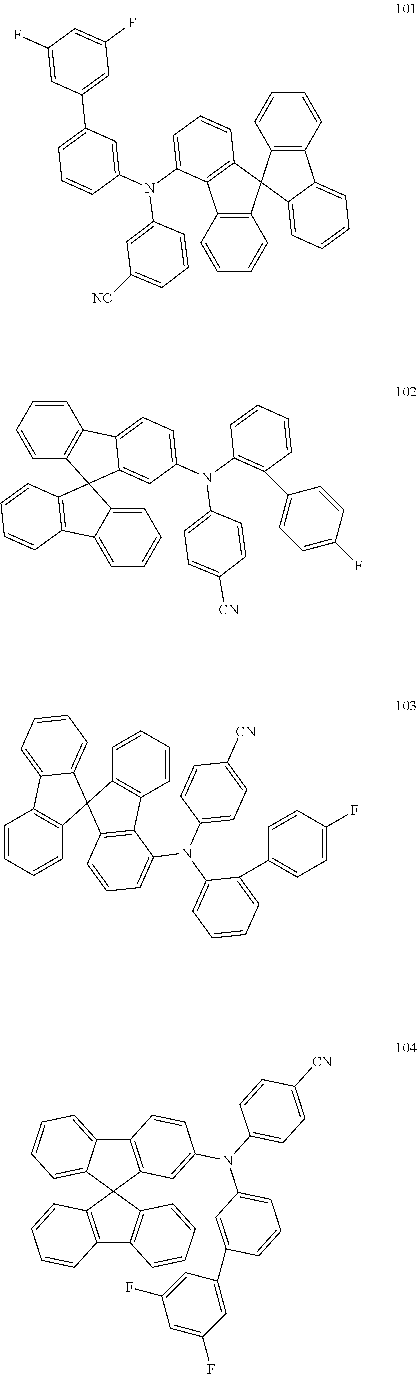 [SMILES: N#Cc1cccc(N(c2cccc(-c3cc(F)cc(F)c3)c2)c2cccc3c2-c2ccccc2C32c3ccccc3-c3ccccc32)c1 ; N#Cc1ccc(N(c2ccc3c(c2)C2(c4ccccc4-c4ccccc42)c2ccccc2-3)c2ccccc2-c2ccc(F)cc2)cc1 ; N#Cc1ccc(N(c2ccccc2-c2ccc(F)cc2)c2cccc3c2-c2ccccc2C32c3ccccc3-c3ccccc32)cc1 ; N#Cc1ccc(N(c2cccc(-c3cc(F)cc(F)c3)c2)c2ccc3c(c2)C2(c4ccccc4-c4ccccc42)c2ccccc2-3)cc1]

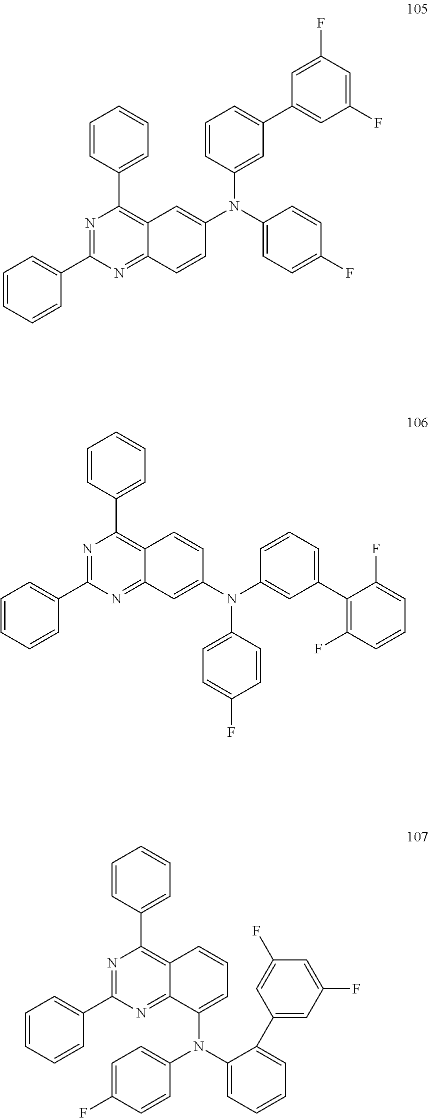 [SMILES: Fc1ccc(N(c2cccc(-c3cc(F)cc(F)c3)c2)c2ccc3nc(-c4ccccc4)nc(-c4ccccc4)c3c2)cc1 ; Fc1ccc(N(c2cccc(-c3c(F)cccc3F)c2)c2ccc3c(-c4ccccc4)nc(-c4ccccc4)nc3c2)cc1 ; Fc1ccc(N(c2ccccc2-c2cc(F)cc(F)c2)c2cccc3c(-c4ccccc4)nc(-c4ccccc4)nc23)cc1]

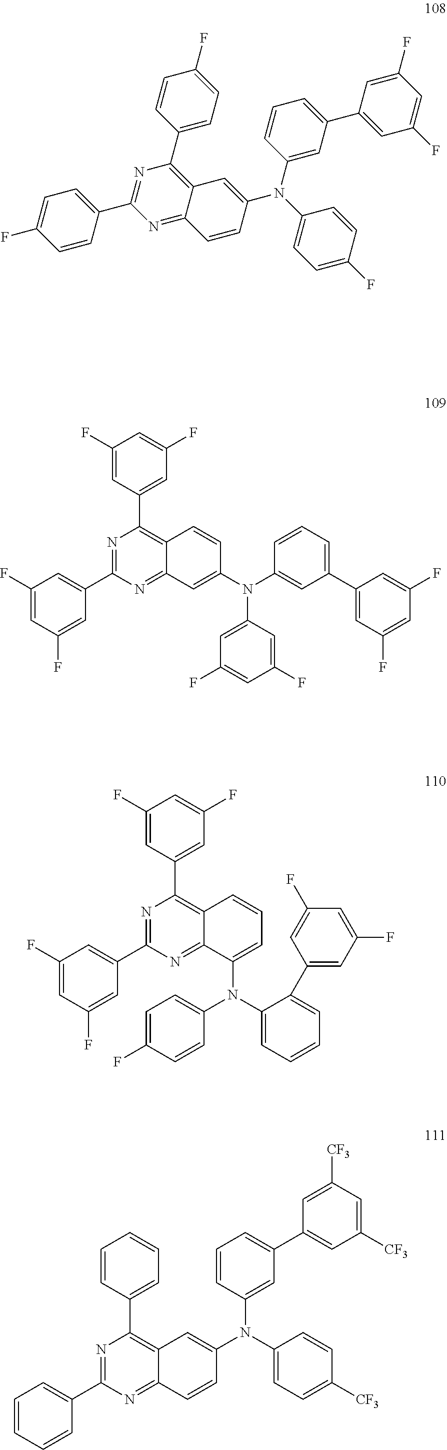 [SMILES: Fc1ccc(-c2nc(-c3ccc(F)cc3)c3cc(N(c4ccc(F)cc4)c4cccc(-c5cc(F)cc(F)c5)c4)ccc3n2)cc1 ; Fc1cc(F)cc(-c2cccc(N(c3cc(F)cc(F)c3)c3ccc4c(-c5cc(F)cc(F)c5)nc(-c5cc(F)cc(F)c5)nc4c3)c2)c1 ; Fc1ccc(N(c2ccccc2-c2cc(F)cc(F)c2)c2cccc3c(-c4cc(F)cc(F)c4)nc(-c4cc(F)cc(F)c4)nc23)cc1 ; FC(F)(F)c1ccc(N(c2cccc(-c3cc(C(F)(F)F)cc(C(F)(F)F)c3)c2)c2ccc3nc(-c4ccccc4)nc(-c4ccccc4)c3c2)cc1]

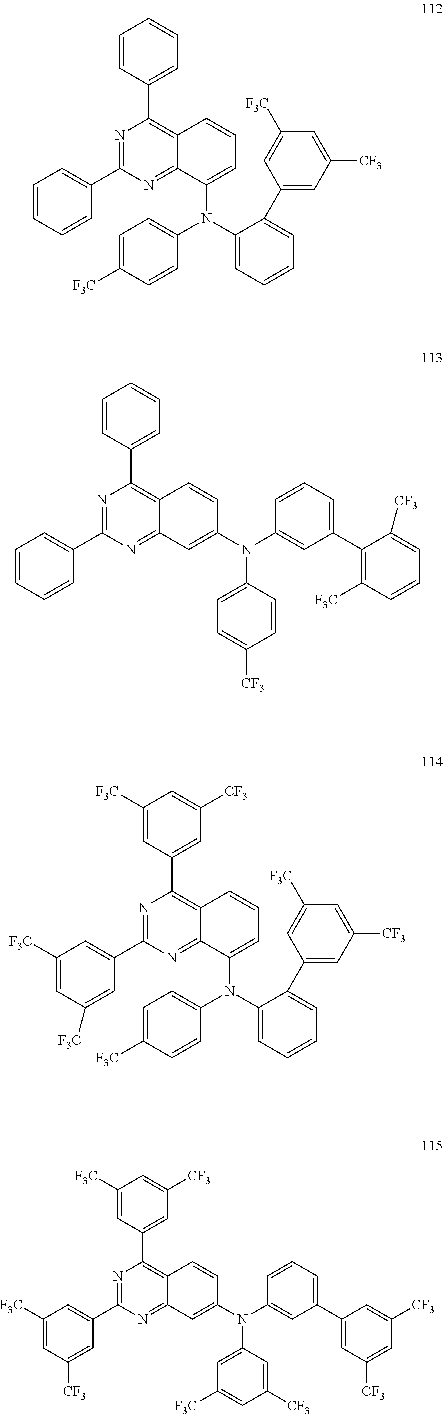 [SMILES: FC(F)(F)c1ccc(N(c2ccccc2-c2cc(C(F)(F)F)cc(C(F)(F)F)c2)c2cccc3c(-c4ccccc4)nc(-c4ccccc4)nc23)cc1 ; FC(F)(F)c1ccc(N(c2cccc(-c3c(C(F)(F)F)cccc3C(F)(F)F)c2)c2ccc3c(-c4ccccc4)nc(-c4ccccc4)nc3c2)cc1 ; FC(F)(F)c1ccc(N(c2ccccc2-c2cc(C(F)(F)F)cc(C(F)(F)F)c2)c2cccc3c(-c4cc(C(F)(F)F)cc(C(F)(F)F)c4)nc(-c4cc(C(F)(F)F)cc(C(F)(F)F)c4)nc23)cc1 ; FC(F)(F)c1cc(-c2cccc(N(c3cc(C(F)(F)F)cc(C(F)(F)F)c3)c3ccc4c(-c5cc(C(F)(F)F)cc(C(F)(F)F)c5)nc(-c5cc(C(F)(F)F)cc(C(F)(F)F)c5)nc4c3)c2)cc(C(F)(F)F)c1]

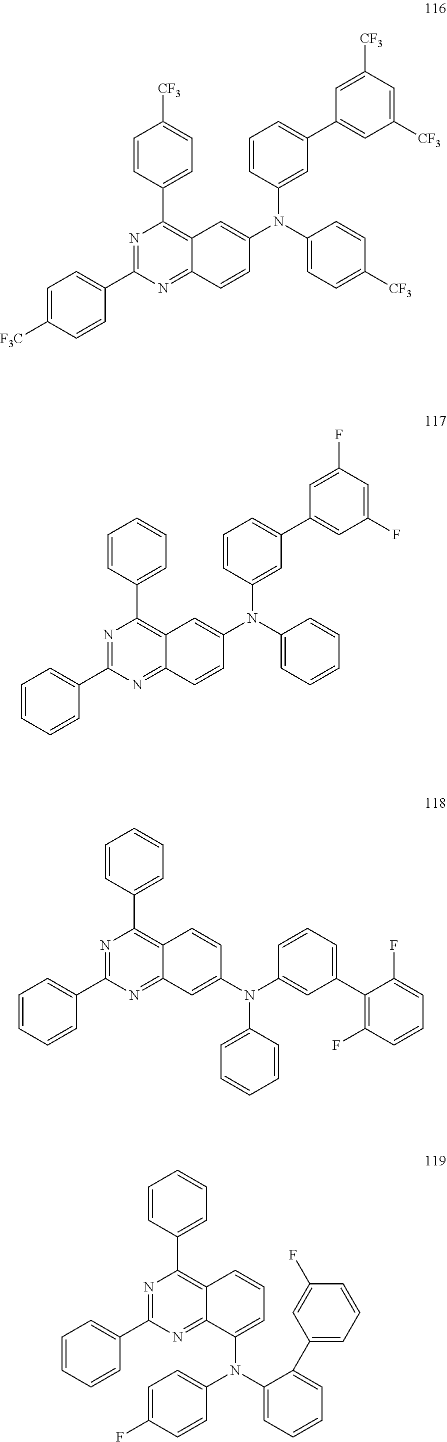 [SMILES: FC(F)(F)c1ccc(-c2nc(-c3ccc(C(F)(F)F)cc3)c3cc(N(c4ccc(C(F)(F)F)cc4)c4cccc(-c5cc(C(F)(F)F)cc(C(F)(F)F)c5)c4)ccc3n2)cc1 ; Fc1cc(F)cc(-c2cccc(N(c3ccccc3)c3ccc4nc(-c5ccccc5)nc(-c5ccccc5)c4c3)c2)c1 ; Fc1cccc(F)c1-c1cccc(N(c2ccccc2)c2ccc3c(-c4ccccc4)nc(-c4ccccc4)nc3c2)c1 ; Fc1ccc(N(c2ccccc2-c2cccc(F)c2)c2cccc3c(-c4ccccc4)nc(-c4ccccc4)nc23)cc1]

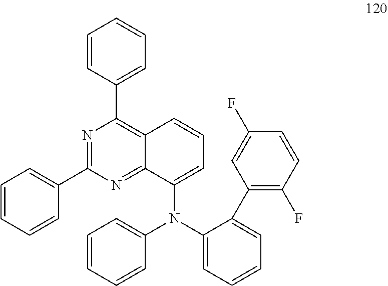 [SMILES: Fc1ccc(F)c(-c2ccccc2N(c2ccccc2)c2cccc3c(-c4ccccc4)nc(-c4ccccc4)nc23)c1]

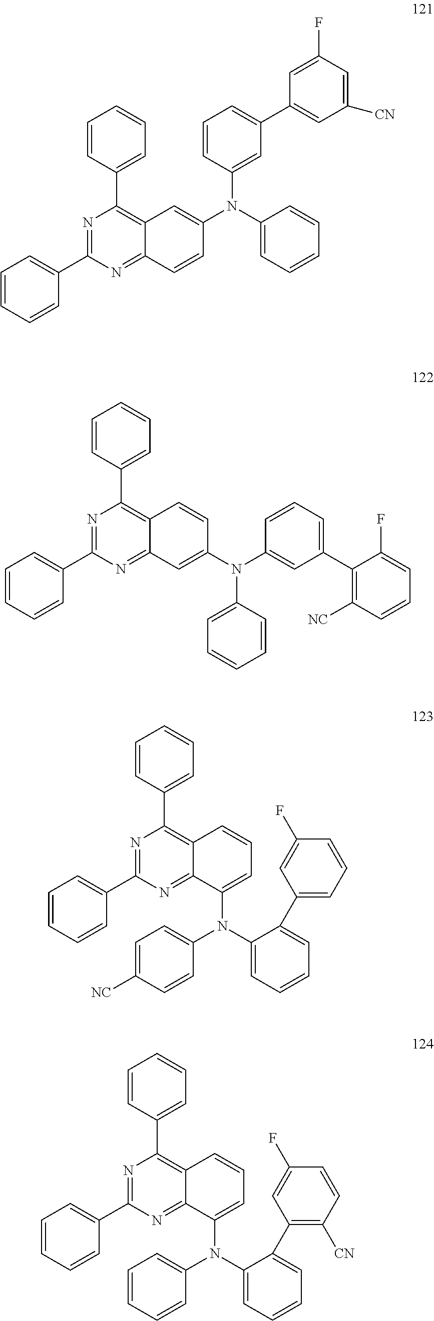 [SMILES: N#Cc1cc(F)cc(-c2cccc(N(c3ccccc3)c3ccc4nc(-c5ccccc5)nc(-c5ccccc5)c4c3)c2)c1 ; N#Cc1cccc(F)c1-c1cccc(N(c2ccccc2)c2ccc3c(-c4ccccc4)nc(-c4ccccc4)nc3c2)c1 ; N#Cc1ccc(N(c2ccccc2-c2cccc(F)c2)c2cccc3c(-c4ccccc4)nc(-c4ccccc4)nc23)cc1 ; N#Cc1ccc(F)cc1-c1ccccc1N(c1ccccc1)c1cccc2c(-c3ccccc3)nc(-c3ccccc3)nc12]

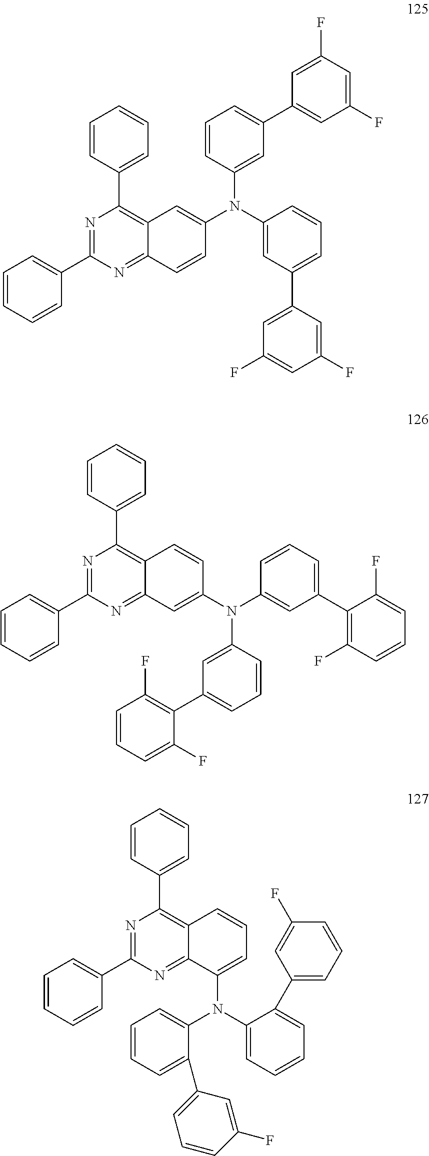 [SMILES: Fc1cc(F)cc(-c2cccc(N(c3cccc(-c4cc(F)cc(F)c4)c3)c3ccc4nc(-c5ccccc5)nc(-c5ccccc5)c4c3)c2)c1 ; Fc1cccc(F)c1-c1cccc(N(c2cccc(-c3c(F)cccc3F)c2)c2ccc3c(-c4ccccc4)nc(-c4ccccc4)nc3c2)c1 ; Fc1cccc(-c2ccccc2N(c2ccccc2-c2cccc(F)c2)c2cccc3c(-c4ccccc4)nc(-c4ccccc4)nc23)c1]

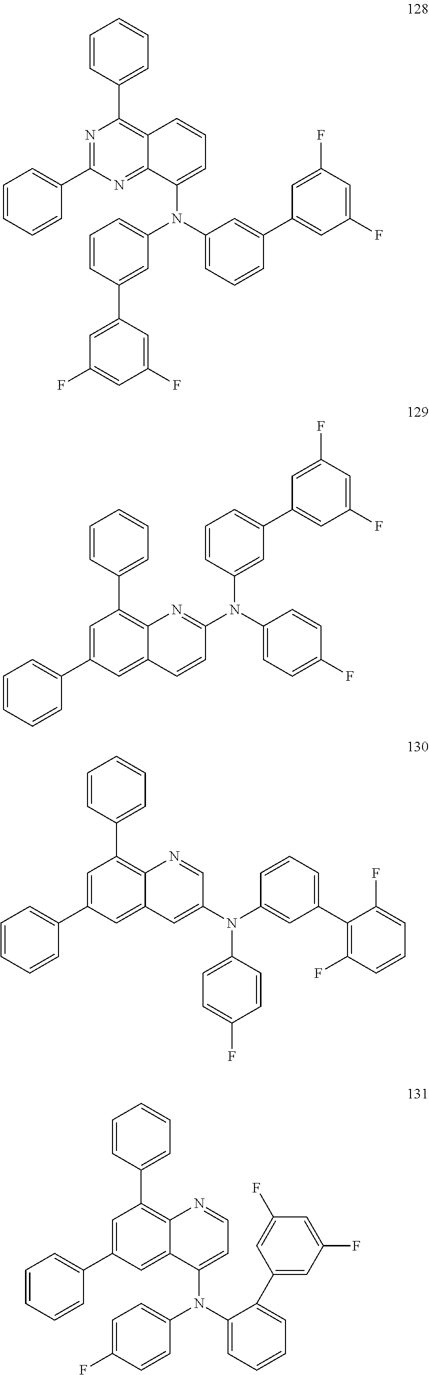 [SMILES: Fc1cc(F)cc(-c2cccc(N(c3cccc(-c4cc(F)cc(F)c4)c3)c3cccc4c(-c5ccccc5)nc(-c5ccccc5)nc34)c2)c1 ; Fc1ccc(N(c2cccc(-c3cc(F)cc(F)c3)c2)c2ccc3cc(-c4ccccc4)cc(-c4ccccc4)c3n2)cc1 ; Fc1ccc(N(c2cccc(-c3c(F)cccc3F)c2)c2cnc3c(-c4ccccc4)cc(-c4ccccc4)cc3c2)cc1 ; Fc1ccc(N(c2ccccc2-c2cc(F)cc(F)c2)c2ccnc3c(-c4ccccc4)cc(-c4ccccc4)cc23)cc1]

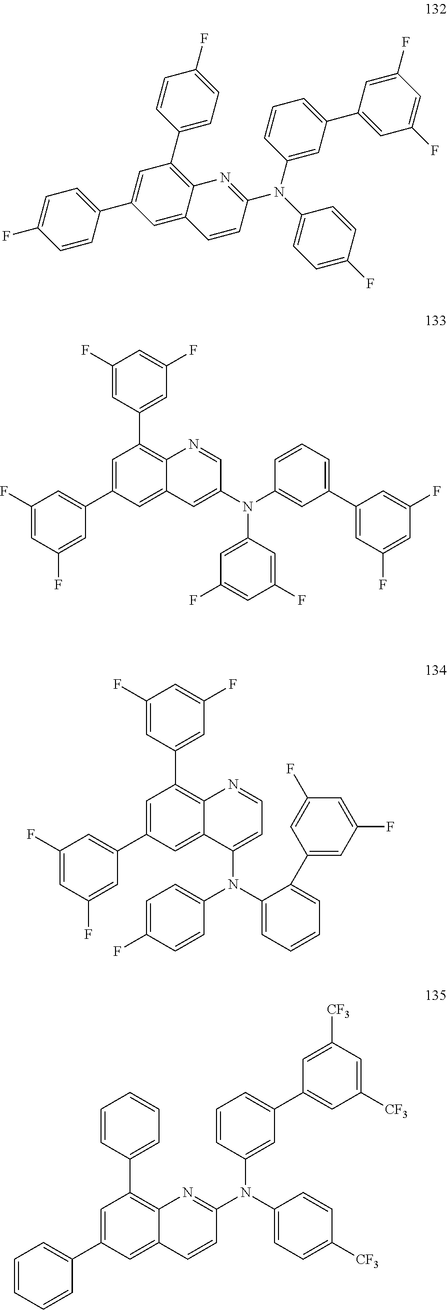 [SMILES: Fc1ccc(-c2cc(-c3ccc(F)cc3)c3nc(N(c4ccc(F)cc4)c4cccc(-c5cc(F)cc(F)c5)c4)ccc3c2)cc1 ; Fc1cc(F)cc(-c2cccc(N(c3cc(F)cc(F)c3)c3cnc4c(-c5cc(F)cc(F)c5)cc(-c5cc(F)cc(F)c5)cc4c3)c2)c1 ; Fc1ccc(N(c2ccccc2-c2cc(F)cc(F)c2)c2ccnc3c(-c4cc(F)cc(F)c4)cc(-c4cc(F)cc(F)c4)cc23)cc1 ; FC(F)(F)c1ccc(N(c2cccc(-c3cc(C(F)(F)F)cc(C(F)(F)F)c3)c2)c2ccc3cc(-c4ccccc4)cc(-c4ccccc4)c3n2)cc1]

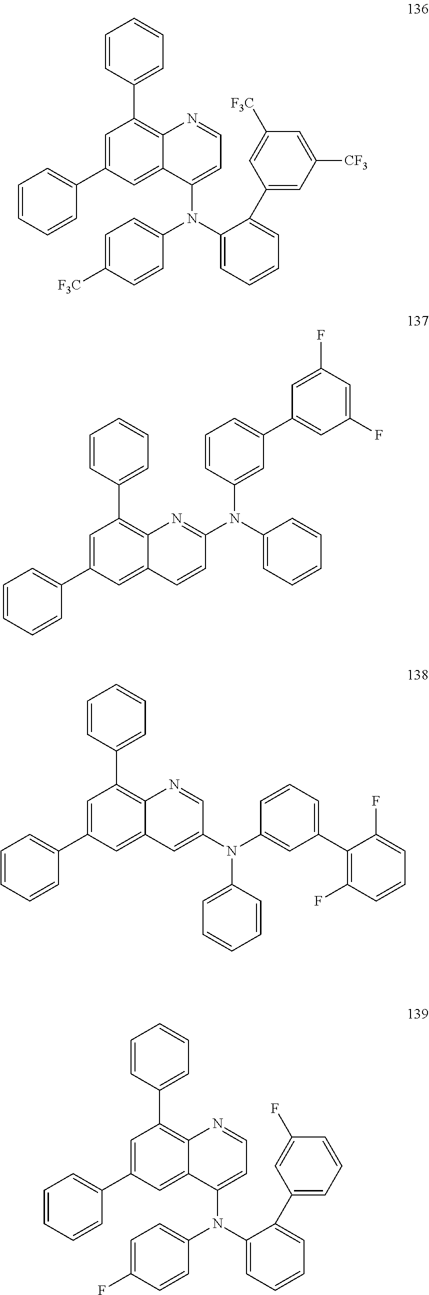 [SMILES: FC(F)(F)c1ccc(N(c2ccccc2-c2cc(C(F)(F)F)cc(C(F)(F)F)c2)c2ccnc3c(-c4ccccc4)cc(-c4ccccc4)cc23)cc1 ; Fc1cc(F)cc(-c2cccc(N(c3ccccc3)c3ccc4cc(-c5ccccc5)cc(-c5ccccc5)c4n3)c2)c1 ; Fc1cccc(F)c1-c1cccc(N(c2ccccc2)c2cnc3c(-c4ccccc4)cc(-c4ccccc4)cc3c2)c1 ; Fc1ccc(N(c2ccccc2-c2cccc(F)c2)c2ccnc3c(-c4ccccc4)cc(-c4ccccc4)cc23)cc1]

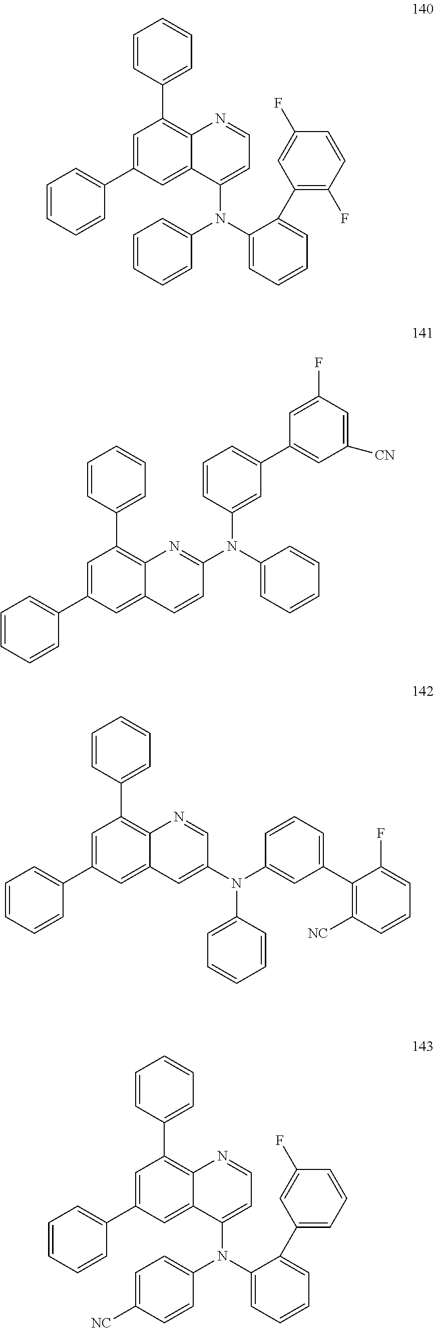 [SMILES: Fc1ccc(F)c(-c2ccccc2N(c2ccccc2)c2ccnc3c(-c4ccccc4)cc(-c4ccccc4)cc23)c1 ; N#Cc1cc(F)cc(-c2cccc(N(c3ccccc3)c3ccc4cc(-c5ccccc5)cc(-c5ccccc5)c4n3)c2)c1 ; N#Cc1cccc(F)c1-c1cccc(N(c2ccccc2)c2cnc3c(-c4ccccc4)cc(-c4ccccc4)cc3c2)c1 ; N#Cc1ccc(N(c2ccccc2-c2cccc(F)c2)c2ccnc3c(-c4ccccc4)cc(-c4ccccc4)cc23)cc1]

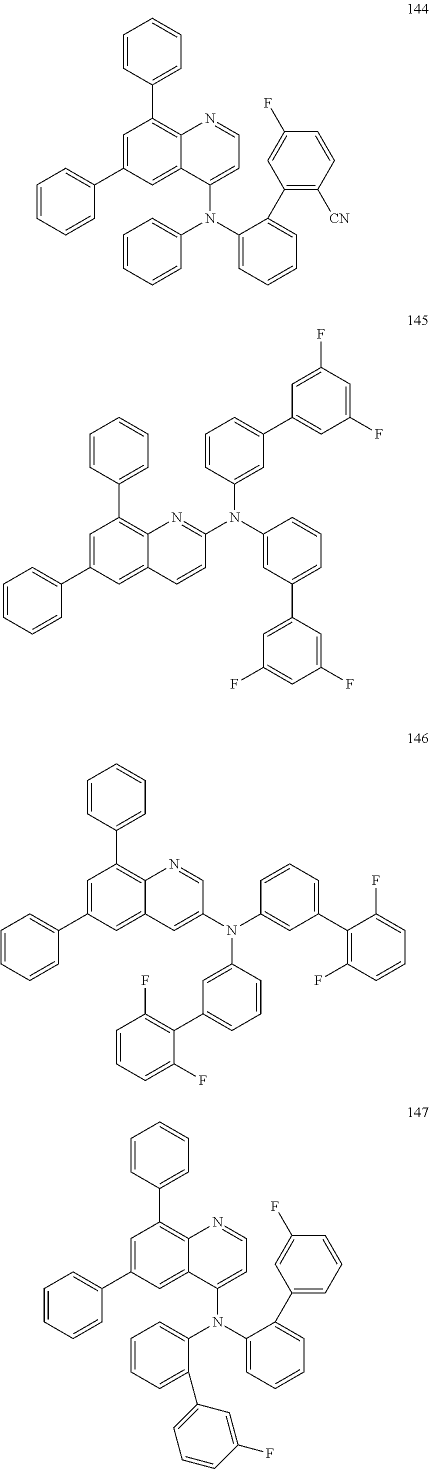

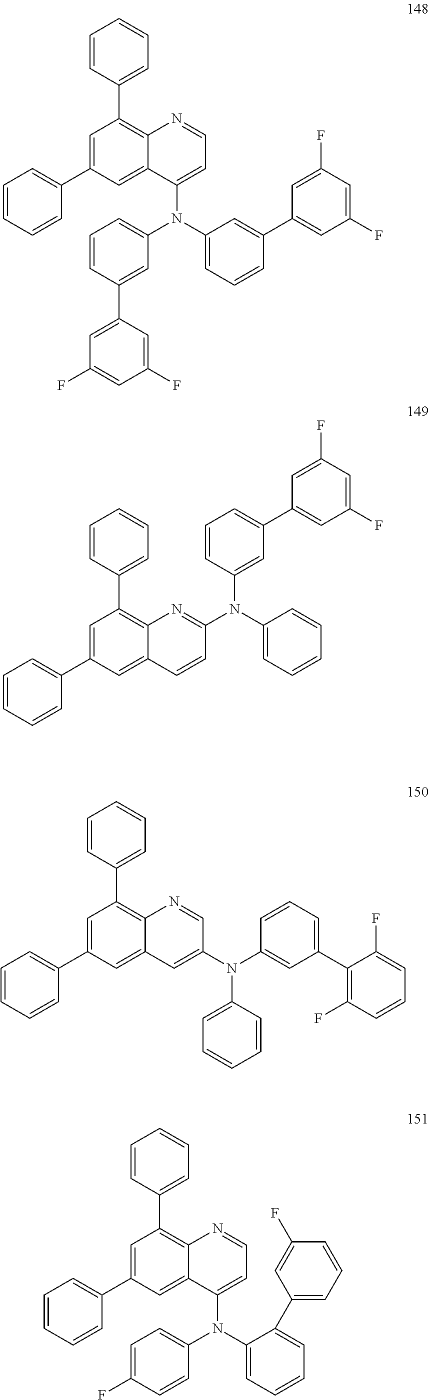 [SMILES: Fc1cc(F)cc(-c2cccc(N(c3cccc(-c4cc(F)cc(F)c4)c3)c3ccnc4c(-c5ccccc5)cc(-c5ccccc5)cc34)c2)c1 ; Fc1cc(F)cc(-c2cccc(N(c3ccccc3)c3ccc4cc(-c5ccccc5)cc(-c5ccccc5)c4n3)c2)c1 ; Fc1cccc(F)c1-c1cccc(N(c2ccccc2)c2cnc3c(-c4ccccc4)cc(-c4ccccc4)cc3c2)c1 ; Fc1ccc(N(c2ccccc2-c2cccc(F)c2)c2ccnc3c(-c4ccccc4)cc(-c4ccccc4)cc23)cc1]

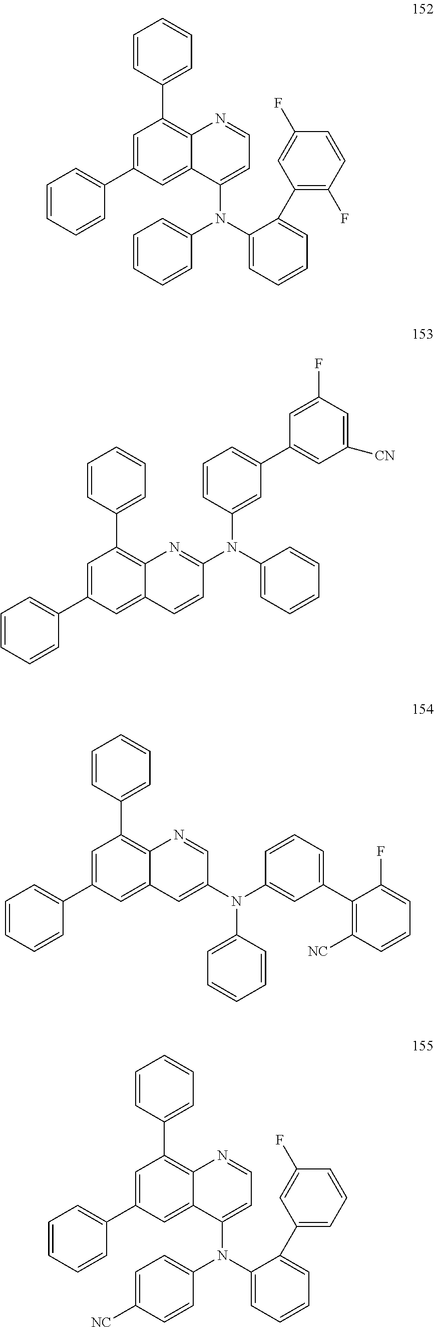 [SMILES: Fc1ccc(F)c(-c2ccccc2N(c2ccccc2)c2ccnc3c(-c4ccccc4)cc(-c4ccccc4)cc23)c1 ; N#Cc1cc(F)cc(-c2cccc(N(c3ccccc3)c3ccc4cc(-c5ccccc5)cc(-c5ccccc5)c4n3)c2)c1 ; N#Cc1cccc(F)c1-c1cccc(N(c2ccccc2)c2cnc3c(-c4ccccc4)cc(-c4ccccc4)cc3c2)c1 ; N#Cc1ccc(N(c2ccccc2-c2cccc(F)c2)c2ccnc3c(-c4ccccc4)cc(-c4ccccc4)cc23)cc1]

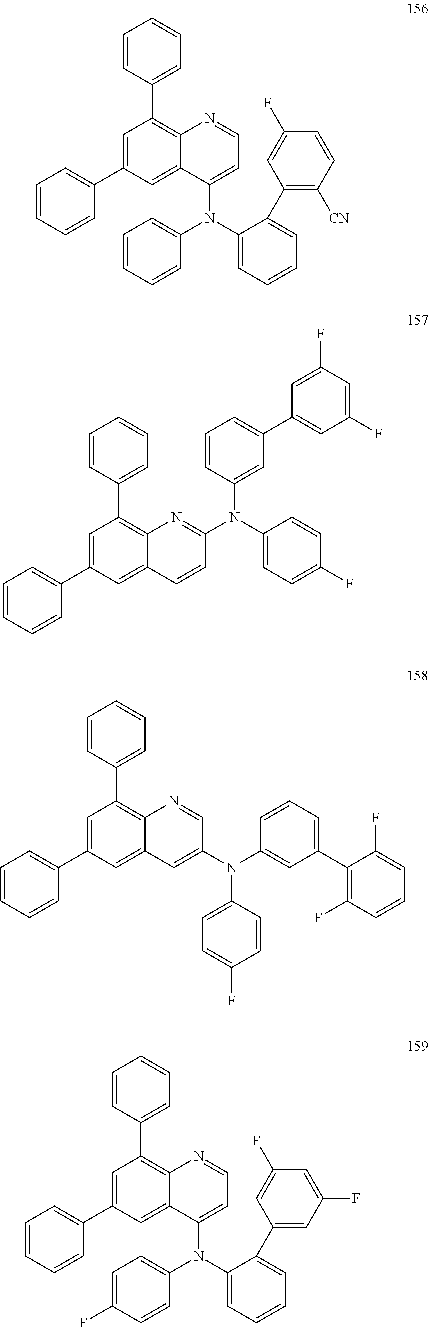 [SMILES: N#Cc1ccc(F)cc1-c1ccccc1N(c1ccccc1)c1ccnc2c(-c3ccccc3)cc(-c3ccccc3)cc12 ; Fc1ccc(N(c2cccc(-c3cc(F)cc(F)c3)c2)c2ccc3cc(-c4ccccc4)cc(-c4ccccc4)c3n2)cc1 ; Fc1ccc(N(c2cccc(-c3c(F)cccc3F)c2)c2cnc3c(-c4ccccc4)cc(-c4ccccc4)cc3c2)cc1 ; Fc1ccc(N(c2ccccc2-c2cc(F)cc(F)c2)c2ccnc3c(-c4ccccc4)cc(-c4ccccc4)cc23)cc1]

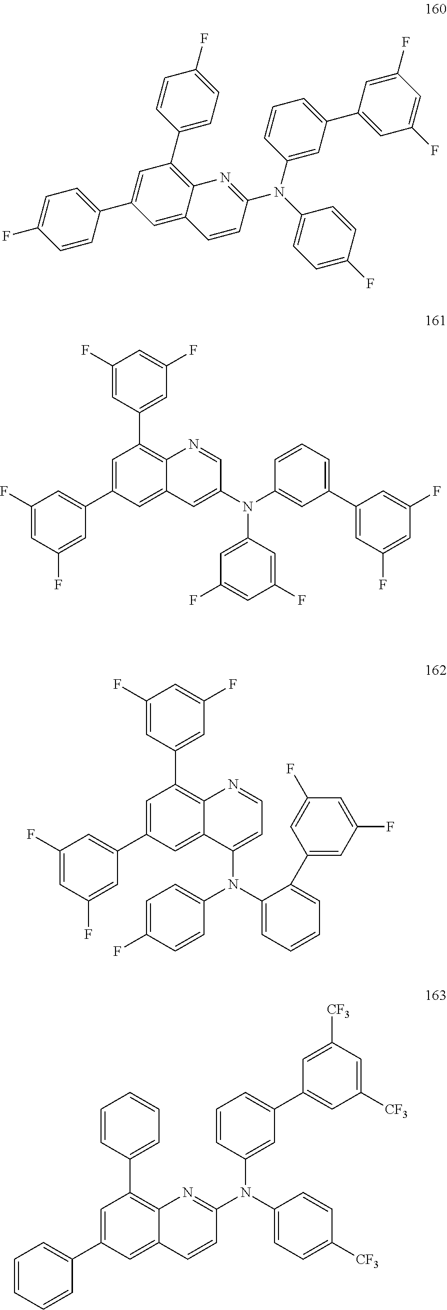 [SMILES: Fc1ccc(-c2cc(-c3ccc(F)cc3)c3nc(N(c4ccc(F)cc4)c4cccc(-c5cc(F)cc(F)c5)c4)ccc3c2)cc1 ; Fc1cc(F)cc(-c2cccc(N(c3cc(F)cc(F)c3)c3cnc4c(-c5cc(F)cc(F)c5)cc(-c5cc(F)cc(F)c5)cc4c3)c2)c1 ; Fc1ccc(N(c2ccccc2-c2cc(F)cc(F)c2)c2ccnc3c(-c4cc(F)cc(F)c4)cc(-c4cc(F)cc(F)c4)cc23)cc1 ; FC(F)(F)c1ccc(N(c2cccc(-c3cc(C(F)(F)F)cc(C(F)(F)F)c3)c2)c2ccc3cc(-c4ccccc4)cc(-c4ccccc4)c3n2)cc1]

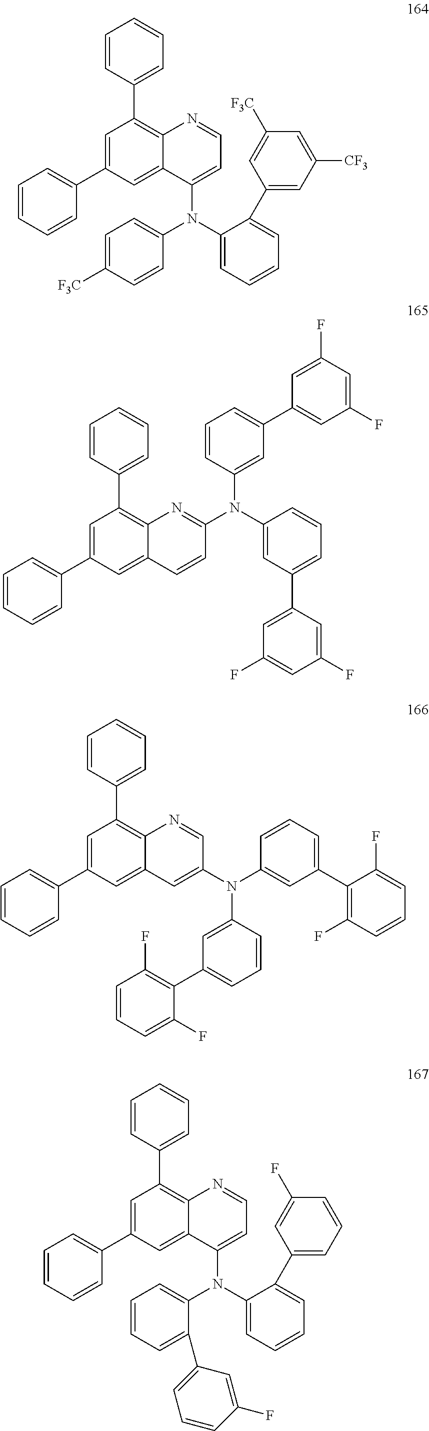 [SMILES: FC(F)(F)c1ccc(N(c2ccccc2-c2cc(C(F)(F)F)cc(C(F)(F)F)c2)c2ccnc3c(-c4ccccc4)cc(-c4ccccc4)cc23)cc1 ; Fc1cc(F)cc(-c2cccc(N(c3cccc(-c4cc(F)cc(F)c4)c3)c3ccc4cc(-c5ccccc5)cc(-c5ccccc5)c4n3)c2)c1 ; Fc1cccc(F)c1-c1cccc(N(c2cccc(-c3c(F)cccc3F)c2)c2cnc3c(-c4ccccc4)cc(-c4ccccc4)cc3c2)c1 ; Fc1cccc(-c2ccccc2N(c2ccccc2-c2cccc(F)c2)c2ccnc3c(-c4ccccc4)cc(-c4ccccc4)cc23)c1]

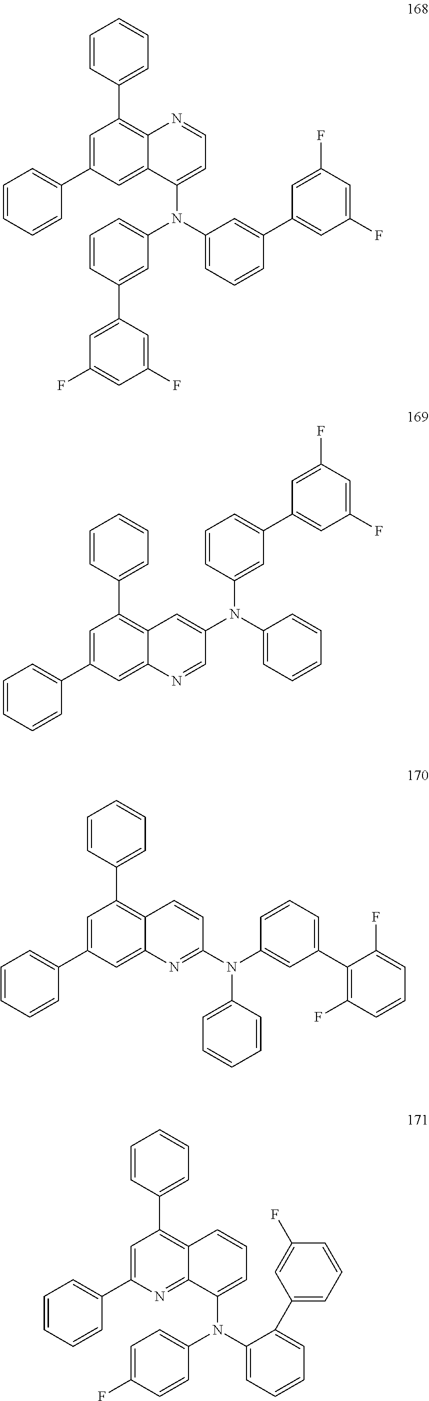 [SMILES: Fc1cc(F)cc(-c2cccc(N(c3cccc(-c4cc(F)cc(F)c4)c3)c3ccnc4c(-c5ccccc5)cc(-c5ccccc5)cc34)c2)c1 ; Fc1cc(F)cc(-c2cccc(N(c3ccccc3)c3cnc4cc(-c5ccccc5)cc(-c5ccccc5)c4c3)c2)c1 ; Fc1cccc(F)c1-c1cccc(N(c2ccccc2)c2ccc3c(-c4ccccc4)cc(-c4ccccc4)cc3n2)c1 ; Fc1ccc(N(c2ccccc2-c2cccc(F)c2)c2cccc3c(-c4ccccc4)cc(-c4ccccc4)nc23)cc1]

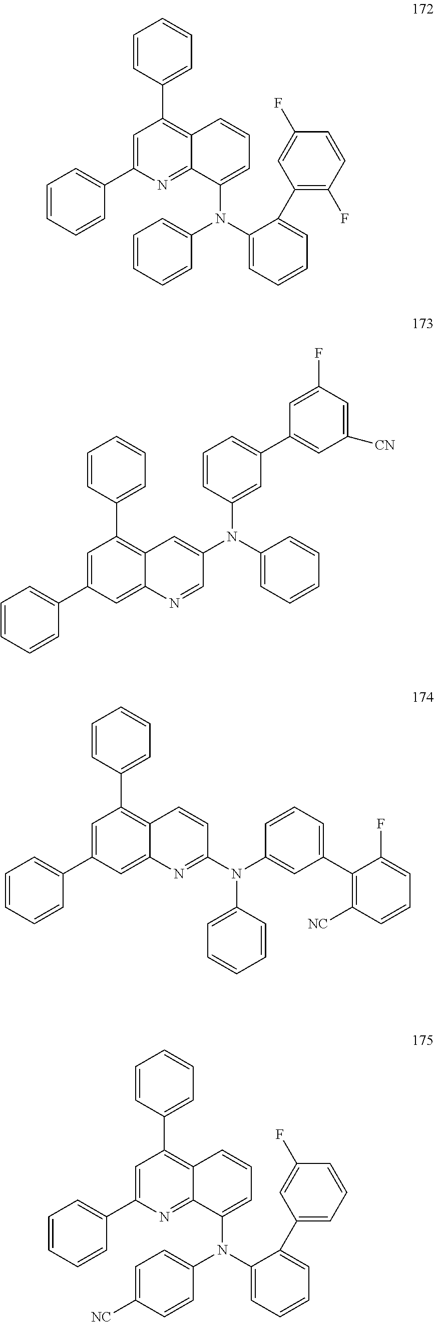 [SMILES: Fc1ccc(F)c(-c2ccccc2N(c2ccccc2)c2cccc3c(-c4ccccc4)cc(-c4ccccc4)nc23)c1 ; N#Cc1cc(F)cc(-c2cccc(N(c3ccccc3)c3cnc4cc(-c5ccccc5)cc(-c5ccccc5)c4c3)c2)c1 ; N#Cc1cccc(F)c1-c1cccc(N(c2ccccc2)c2ccc3c(-c4ccccc4)cc(-c4ccccc4)cc3n2)c1 ; N#Cc1ccc(N(c2ccccc2-c2cccc(F)c2)c2cccc3c(-c4ccccc4)cc(-c4ccccc4)nc23)cc1]

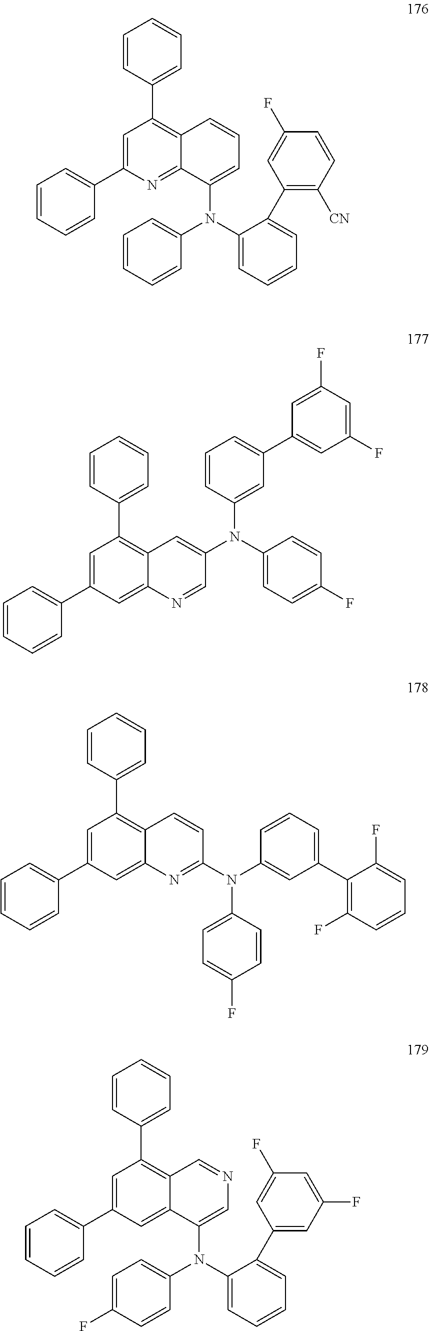 [SMILES: N#Cc1ccc(F)cc1-c1ccccc1N(c1ccccc1)c1cccc2c(-c3ccccc3)cc(-c3ccccc3)nc12 ; Fc1ccc(N(c2cccc(-c3cc(F)cc(F)c3)c2)c2cnc3cc(-c4ccccc4)cc(-c4ccccc4)c3c2)cc1 ; Fc1ccc(N(c2cccc(-c3c(F)cccc3F)c2)c2ccc3c(-c4ccccc4)cc(-c4ccccc4)cc3n2)cc1 ; Fc1ccc(N(c2ccccc2-c2cc(F)cc(F)c2)c2cncc3c(-c4ccccc4)cc(-c4ccccc4)cc23)cc1]

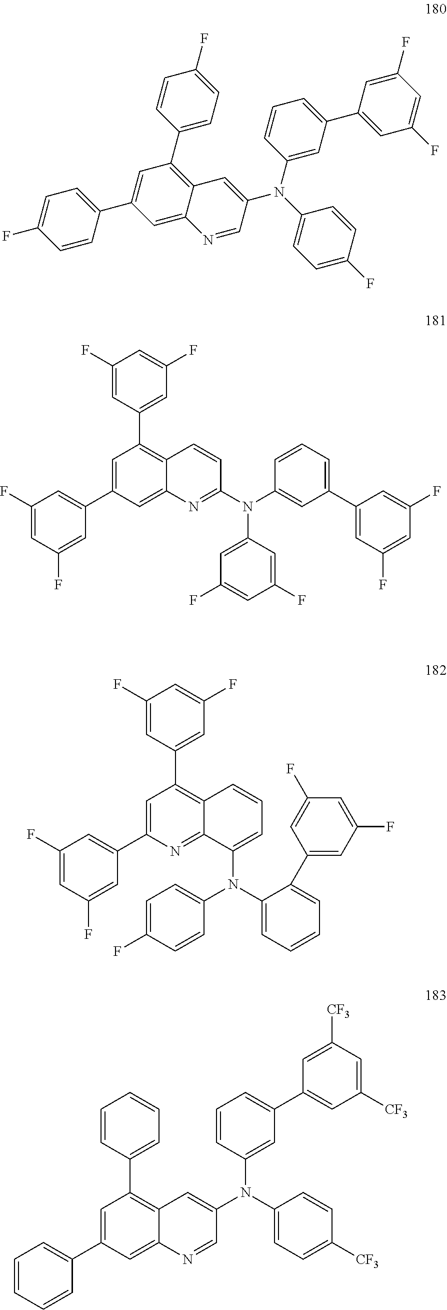 [SMILES: Fc1ccc(-c2cc(-c3ccc(F)cc3)c3cc(N(c4ccc(F)cc4)c4cccc(-c5cc(F)cc(F)c5)c4)cnc3c2)cc1 ; Fc1cc(F)cc(-c2cccc(N(c3cc(F)cc(F)c3)c3ccc4c(-c5cc(F)cc(F)c5)cc(-c5cc(F)cc(F)c5)cc4n3)c2)c1 ; Fc1ccc(N(c2ccccc2-c2cc(F)cc(F)c2)c2cccc3c(-c4cc(F)cc(F)c4)cc(-c4cc(F)cc(F)c4)nc23)cc1 ; FC(F)(F)c1ccc(N(c2cccc(-c3cc(C(F)(F)F)cc(C(F)(F)F)c3)c2)c2cnc3cc(-c4ccccc4)cc(-c4ccccc4)c3c2)cc1]

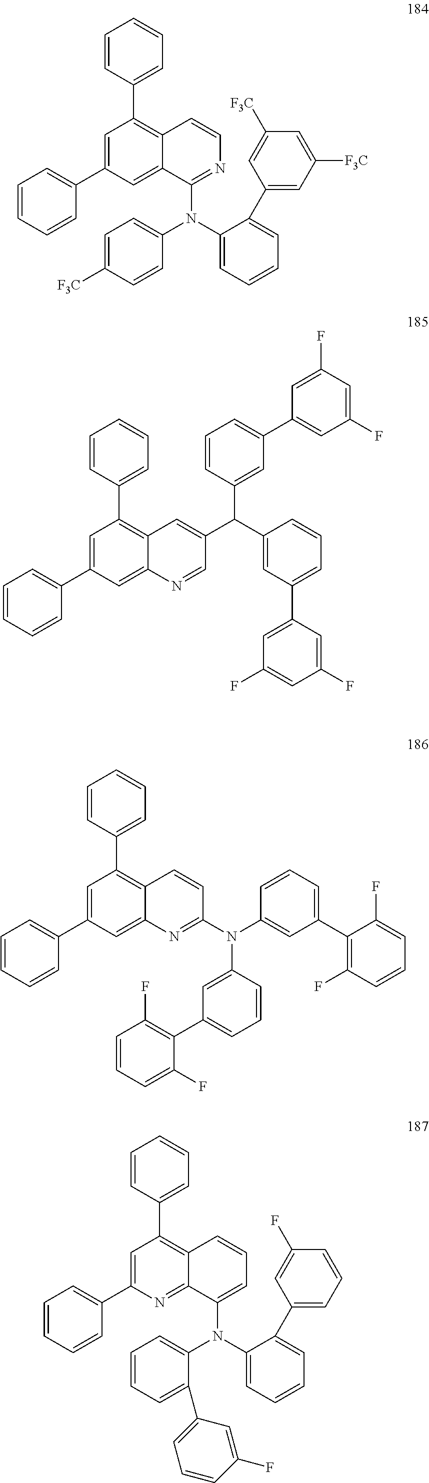 [SMILES: FC(F)(F)c1ccc(N(c2ccccc2-c2cc(C(F)(F)F)cc(C(F)(F)F)c2)c2nccc3c(-c4ccccc4)cc(-c4ccccc4)cc23)cc1 ; Fc1cc(F)cc(-c2cccc(C(c3cccc(-c4cc(F)cc(F)c4)c3)c3cnc4cc(-c5ccccc5)cc(-c5ccccc5)c4c3)c2)c1 ; Fc1cccc(F)c1-c1cccc(N(c2cccc(-c3c(F)cccc3F)c2)c2ccc3c(-c4ccccc4)cc(-c4ccccc4)cc3n2)c1 ; Fc1cccc(-c2ccccc2N(c2ccccc2-c2cccc(F)c2)c2cccc3c(-c4ccccc4)cc(-c4ccccc4)nc23)c1]

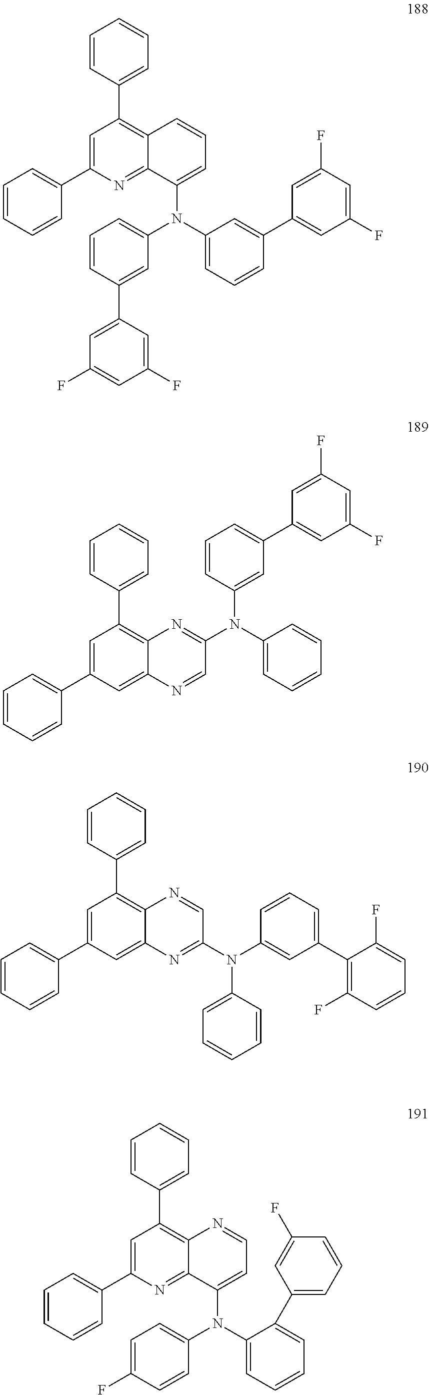 [SMILES: Fc1cc(F)cc(-c2cccc(N(c3cccc(-c4cc(F)cc(F)c4)c3)c3cccc4c(-c5ccccc5)cc(-c5ccccc5)nc34)c2)c1 ; Fc1cc(F)cc(-c2cccc(N(c3ccccc3)c3cnc4cc(-c5ccccc5)cc(-c5ccccc5)c4n3)c2)c1 ; Fc1cccc(F)c1-c1cccc(N(c2ccccc2)c2cnc3c(-c4ccccc4)cc(-c4ccccc4)cc3n2)c1 ; Fc1ccc(N(c2ccccc2-c2cccc(F)c2)c2ccnc3c(-c4ccccc4)cc(-c4ccccc4)nc23)cc1]

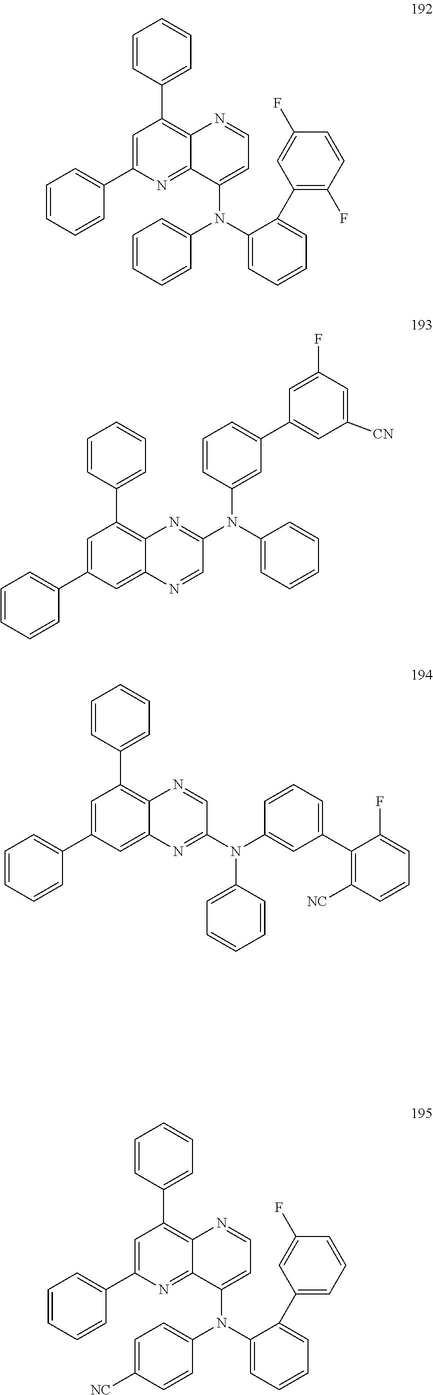 [SMILES: Fc1ccc(F)c(-c2ccccc2N(c2ccccc2)c2ccnc3c(-c4ccccc4)cc(-c4ccccc4)nc23)c1 ; N#Cc1cc(F)cc(-c2cccc(N(c3ccccc3)c3cnc4cc(-c5ccccc5)cc(-c5ccccc5)c4n3)c2)c1 ; N#Cc1cccc(F)c1-c1cccc(N(c2ccccc2)c2cnc3c(-c4ccccc4)cc(-c4ccccc4)cc3n2)c1 ; N#Cc1ccc(N(c2ccccc2-c2cccc(F)c2)c2ccnc3c(-c4ccccc4)cc(-c4ccccc4)nc23)cc1]

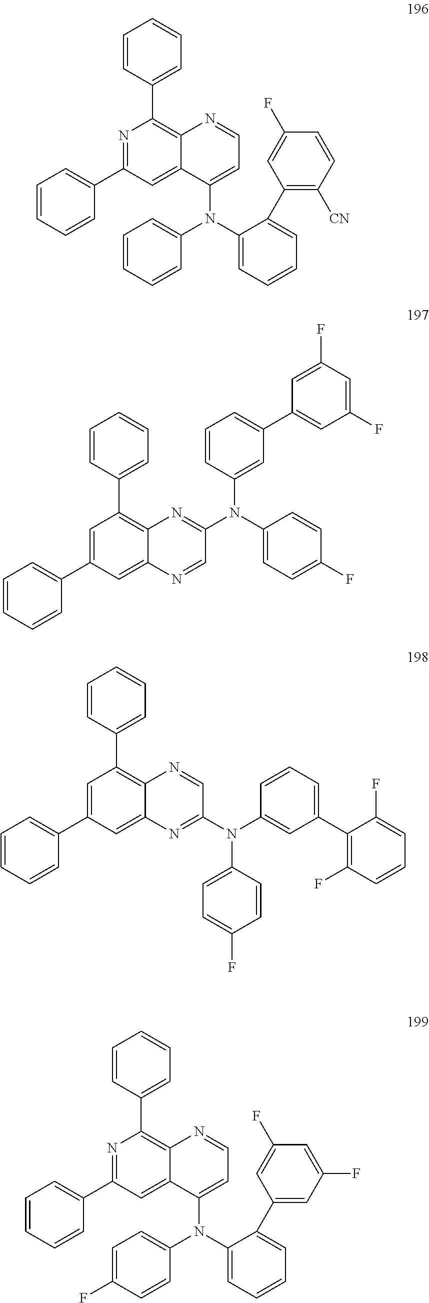 [SMILES: N#Cc1ccc(F)cc1-c1ccccc1N(c1ccccc1)c1ccnc2c(-c3ccccc3)nc(-c3ccccc3)cc12 ; Fc1ccc(N(c2cccc(-c3cc(F)cc(F)c3)c2)c2cnc3cc(-c4ccccc4)cc(-c4ccccc4)c3n2)cc1 ; Fc1ccc(N(c2cccc(-c3c(F)cccc3F)c2)c2cnc3c(-c4ccccc4)cc(-c4ccccc4)cc3n2)cc1 ; Fc1ccc(N(c2ccccc2-c2cc(F)cc(F)c2)c2ccnc3c(-c4ccccc4)nc(-c4ccccc4)cc23)cc1]

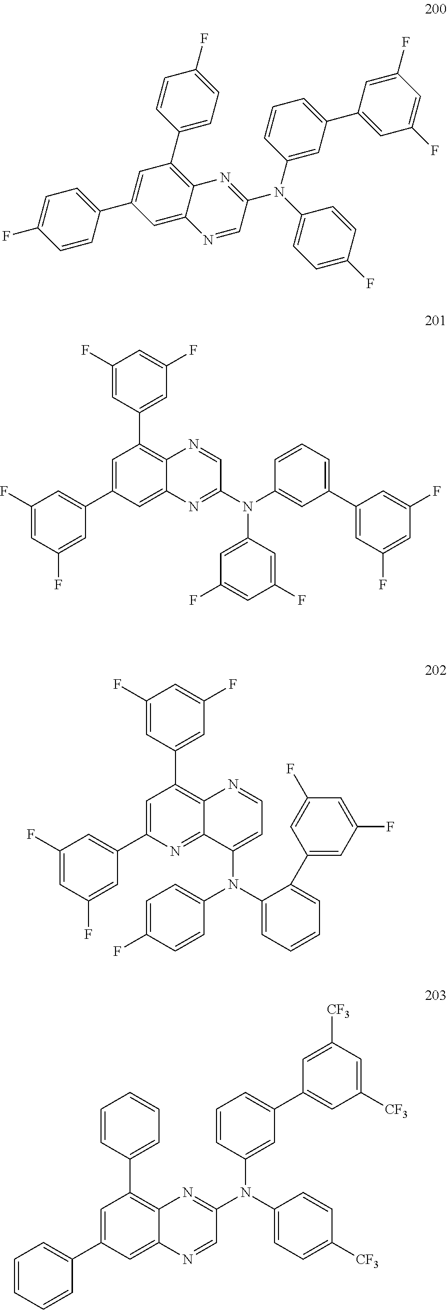 [SMILES: Fc1ccc(-c2cc(-c3ccc(F)cc3)c3nc(N(c4ccc(F)cc4)c4cccc(-c5cc(F)cc(F)c5)c4)cnc3c2)cc1 ; Fc1cc(F)cc(-c2cccc(N(c3cc(F)cc(F)c3)c3cnc4c(-c5cc(F)cc(F)c5)cc(-c5cc(F)cc(F)c5)cc4n3)c2)c1 ; Fc1ccc(N(c2ccccc2-c2cc(F)cc(F)c2)c2ccnc3c(-c4cc(F)cc(F)c4)cc(-c4cc(F)cc(F)c4)nc23)cc1 ; FC(F)(F)c1ccc(N(c2cccc(-c3cc(C(F)(F)F)cc(C(F)(F)F)c3)c2)c2cnc3cc(-c4ccccc4)cc(-c4ccccc4)c3n2)cc1]

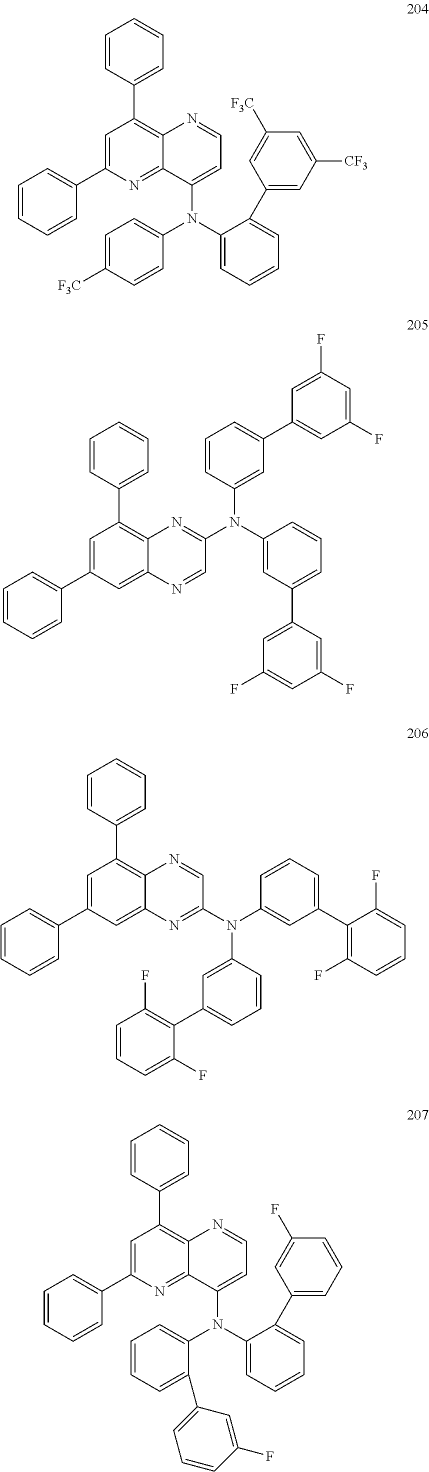 [SMILES: FC(F)(F)c1ccc(N(c2ccccc2-c2cc(C(F)(F)F)cc(C(F)(F)F)c2)c2ccnc3c(-c4ccccc4)cc(-c4ccccc4)nc23)cc1 ; Fc1cc(F)cc(-c2cccc(N(c3cccc(-c4cc(F)cc(F)c4)c3)c3cnc4cc(-c5ccccc5)cc(-c5ccccc5)c4n3)c2)c1 ; Fc1cccc(F)c1-c1cccc(N(c2cccc(-c3c(F)cccc3F)c2)c2cnc3c(-c4ccccc4)cc(-c4ccccc4)cc3n2)c1 ; Fc1cccc(-c2ccccc2N(c2ccccc2-c2cccc(F)c2)c2ccnc3c(-c4ccccc4)cc(-c4ccccc4)nc23)c1]

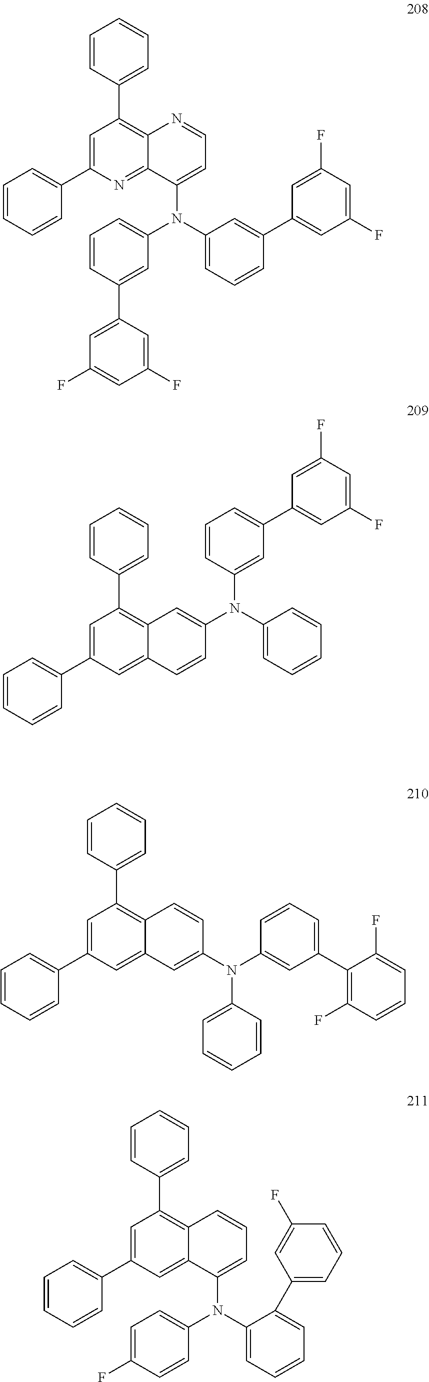 [SMILES: Fc1cc(F)cc(-c2cccc(N(c3cccc(-c4cc(F)cc(F)c4)c3)c3ccnc4c(-c5ccccc5)cc(-c5ccccc5)nc34)c2)c1 ; Fc1cc(F)cc(-c2cccc(N(c3ccccc3)c3ccc4cc(-c5ccccc5)cc(-c5ccccc5)c4c3)c2)c1 ; Fc1cccc(F)c1-c1cccc(N(c2ccccc2)c2ccc3c(-c4ccccc4)cc(-c4ccccc4)cc3c2)c1 ; Fc1ccc(N(c2ccccc2-c2cccc(F)c2)c2cccc3c(-c4ccccc4)cc(-c4ccccc4)cc23)cc1]

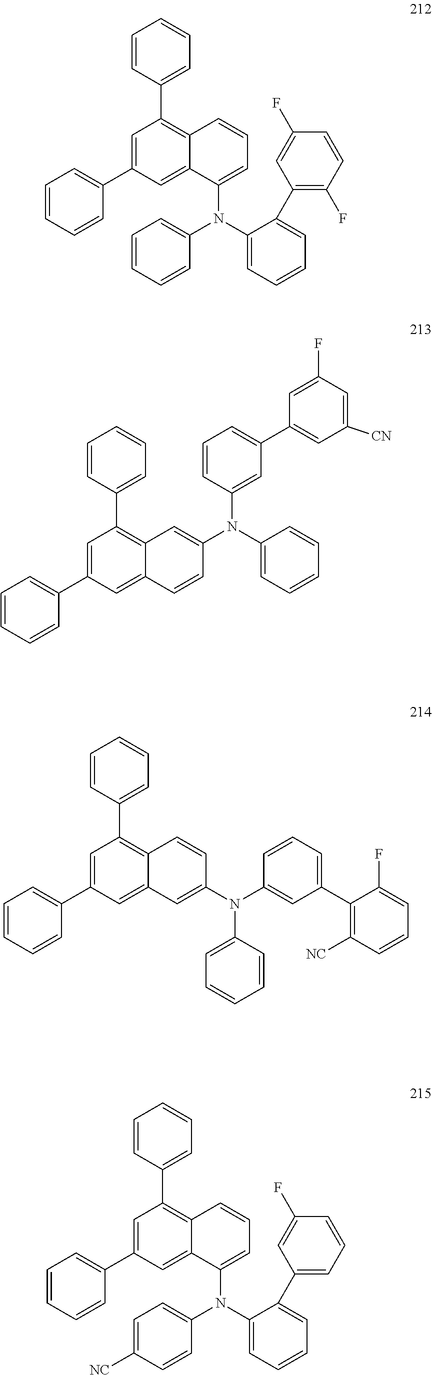 [SMILES: Fc1ccc(F)c(-c2ccccc2N(c2ccccc2)c2cccc3c(-c4ccccc4)cc(-c4ccccc4)cc23)c1 ; N#Cc1cc(F)cc(-c2cccc(N(c3ccccc3)c3ccc4cc(-c5ccccc5)cc(-c5ccccc5)c4c3)c2)c1 ; N#Cc1cccc(F)c1-c1cccc(N(c2ccccc2)c2ccc3c(-c4ccccc4)cc(-c4ccccc4)cc3c2)c1 ; N#Cc1ccc(N(c2ccccc2-c2cccc(F)c2)c2cccc3c(-c4ccccc4)cc(-c4ccccc4)cc23)cc1]

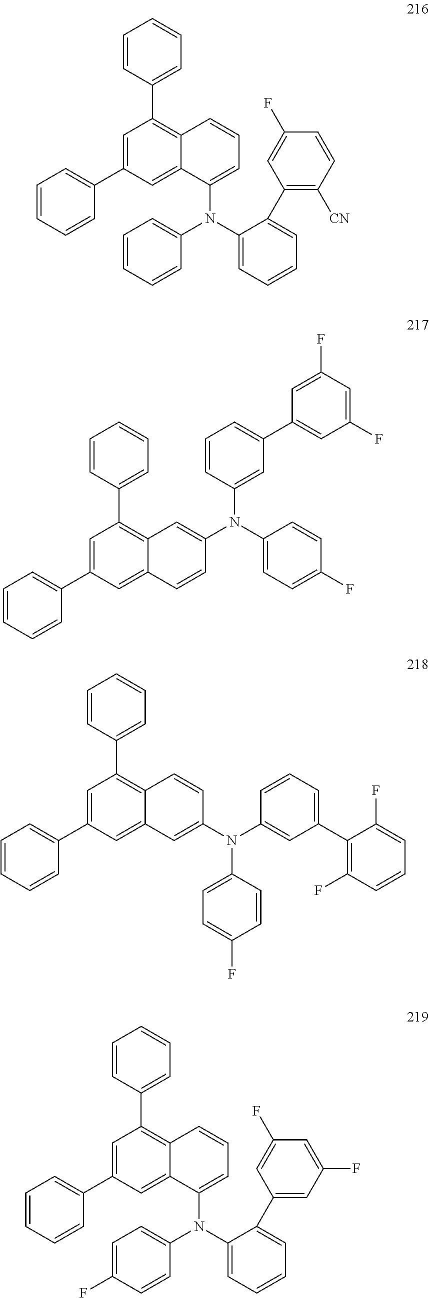 [SMILES: N#Cc1ccc(F)cc1-c1ccccc1N(c1ccccc1)c1cccc2c(-c3ccccc3)cc(-c3ccccc3)cc12 ; Fc1ccc(N(c2cccc(-c3cc(F)cc(F)c3)c2)c2ccc3cc(-c4ccccc4)cc(-c4ccccc4)c3c2)cc1 ; Fc1ccc(N(c2cccc(-c3c(F)cccc3F)c2)c2ccc3c(-c4ccccc4)cc(-c4ccccc4)cc3c2)cc1 ; Fc1ccc(N(c2ccccc2-c2cc(F)cc(F)c2)c2cccc3c(-c4ccccc4)cc(-c4ccccc4)cc23)cc1]

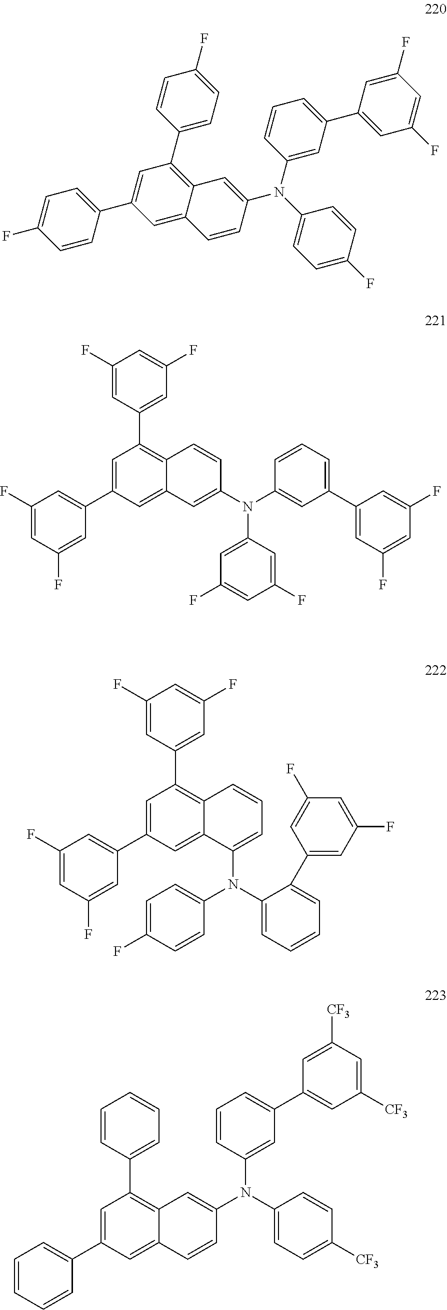 [SMILES: Fc1ccc(-c2cc(-c3ccc(F)cc3)c3cc(N(c4ccc(F)cc4)c4cccc(-c5cc(F)cc(F)c5)c4)ccc3c2)cc1 ; Fc1cc(F)cc(-c2cccc(N(c3cc(F)cc(F)c3)c3ccc4c(-c5cc(F)cc(F)c5)cc(-c5cc(F)cc(F)c5)cc4c3)c2)c1 ; Fc1ccc(N(c2ccccc2-c2cc(F)cc(F)c2)c2cccc3c(-c4cc(F)cc(F)c4)cc(-c4cc(F)cc(F)c4)cc23)cc1 ; FC(F)(F)c1ccc(N(c2cccc(-c3cc(C(F)(F)F)cc(C(F)(F)F)c3)c2)c2ccc3cc(-c4ccccc4)cc(-c4ccccc4)c3c2)cc1]

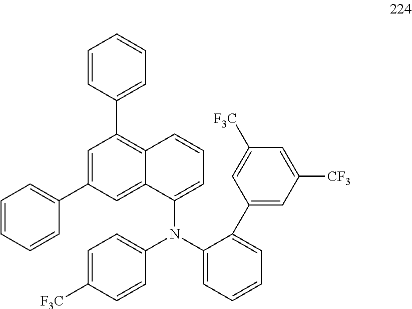 [SMILES: FC(F)(F)c1ccc(N(c2ccccc2-c2cc(C(F)(F)F)cc(C(F)(F)F)c2)c2cccc3c(-c4ccccc4)cc(-c4ccccc4)cc23)cc1]

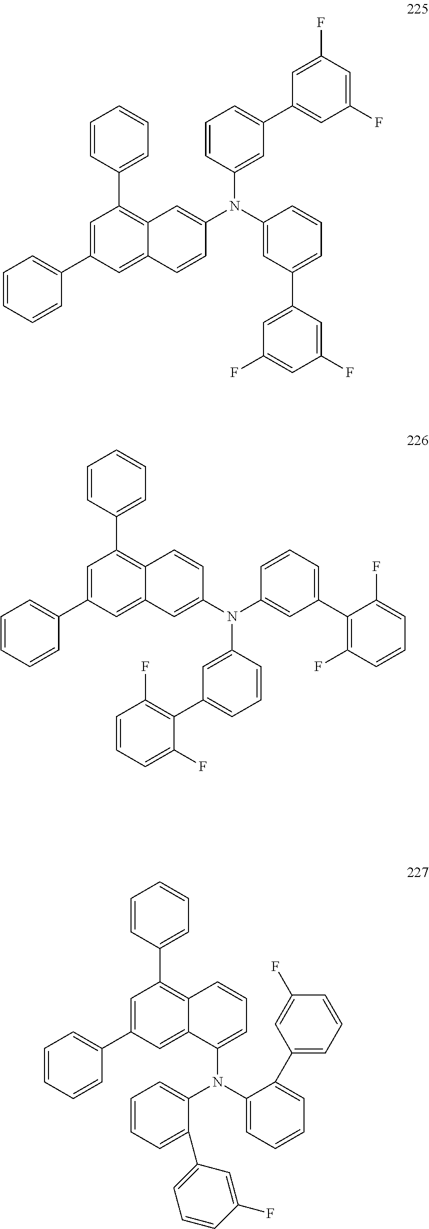 [SMILES: Fc1cc(F)cc(-c2cccc(N(c3cccc(-c4cc(F)cc(F)c4)c3)c3ccc4cc(-c5ccccc5)cc(-c5ccccc5)c4c3)c2)c1 ; Fc1cccc(F)c1-c1cccc(N(c2cccc(-c3c(F)cccc3F)c2)c2ccc3c(-c4ccccc4)cc(-c4ccccc4)cc3c2)c1 ; Fc1cccc(-c2ccccc2N(c2ccccc2-c2cccc(F)c2)c2cccc3c(-c4ccccc4)cc(-c4ccccc4)cc23)c1]

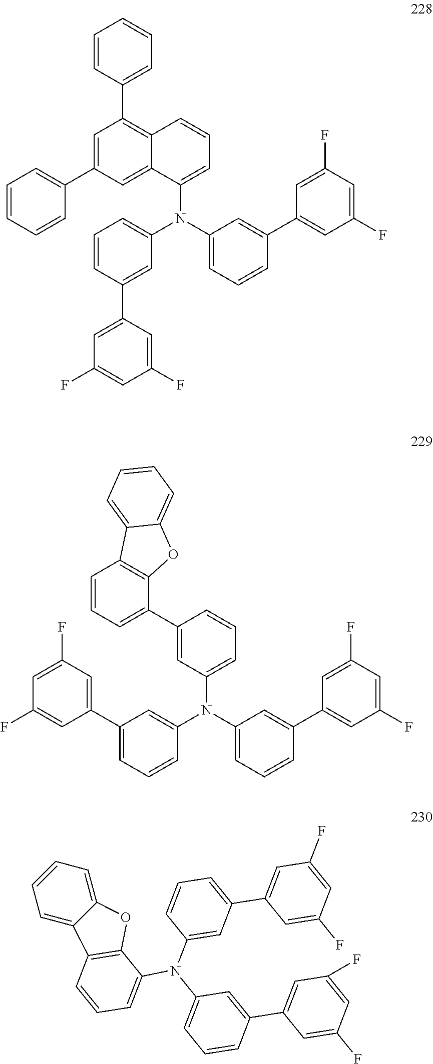 [SMILES: Fc1cc(F)cc(-c2cccc(N(c3cccc(-c4cc(F)cc(F)c4)c3)c3cccc4c(-c5ccccc5)cc(-c5ccccc5)cc34)c2)c1 ; Fc1cc(F)cc(-c2cccc(N(c3cccc(-c4cc(F)cc(F)c4)c3)c3cccc(-c4cccc5c4oc4ccccc45)c3)c2)c1 ; Fc1cc(F)cc(-c2cccc(N(c3cccc(-c4cc(F)cc(F)c4)c3)c3cccc4c3oc3ccccc34)c2)c1]

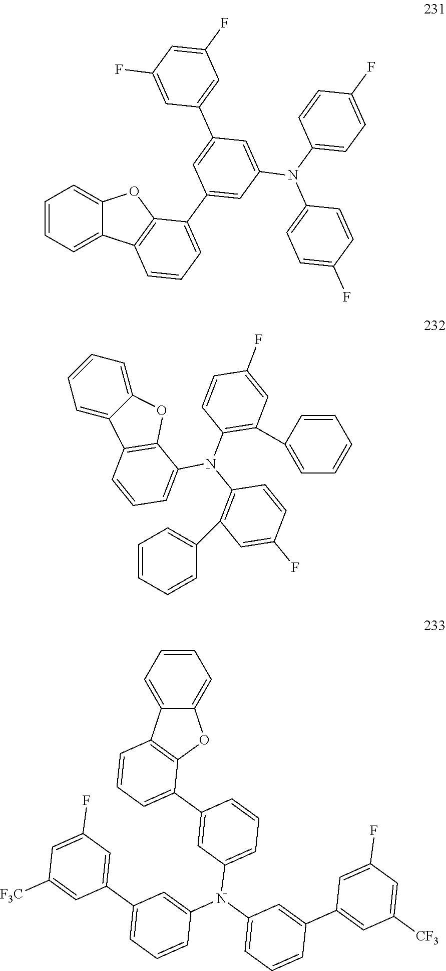 [SMILES: Fc1ccc(N(c2ccc(F)cc2)c2cc(-c3cc(F)cc(F)c3)cc(-c3cccc4c3oc3ccccc34)c2)cc1 ; Fc1ccc(N(c2ccc(F)cc2-c2ccccc2)c2cccc3c2oc2ccccc23)c(-c2ccccc2)c1 ; Fc1cc(-c2cccc(N(c3cccc(-c4cc(F)cc(C(F)(F)F)c4)c3)c3cccc(-c4cccc5c4oc4ccccc45)c3)c2)cc(C(F)(F)F)c1]

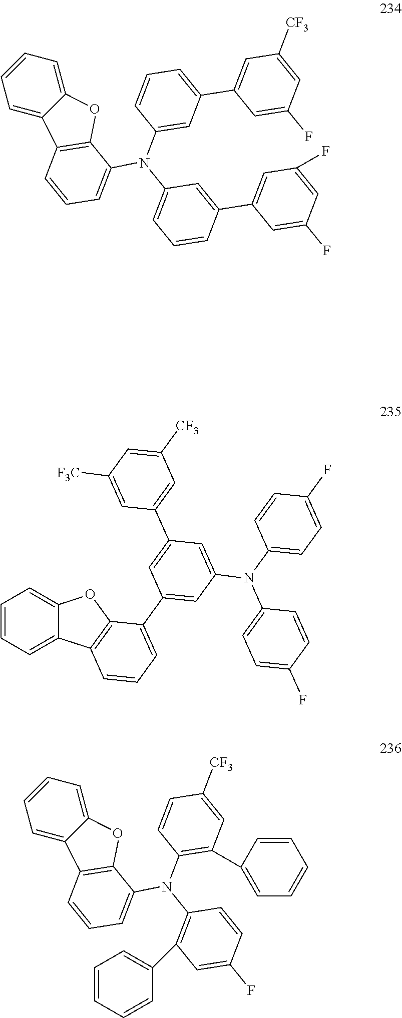 [SMILES: Fc1cc(F)cc(-c2cccc(N(c3cccc(-c4cc(F)cc(C(F)(F)F)c4)c3)c3cccc4c3oc3ccccc34)c2)c1 ; Fc1ccc(N(c2ccc(F)cc2)c2cc(-c3cc(C(F)(F)F)cc(C(F)(F)F)c3)cc(-c3cccc4c3oc3ccccc34)c2)cc1 ; Fc1ccc(N(c2ccc(C(F)(F)F)cc2-c2ccccc2)c2cccc3c2oc2ccccc23)c(-c2ccccc2)c1]

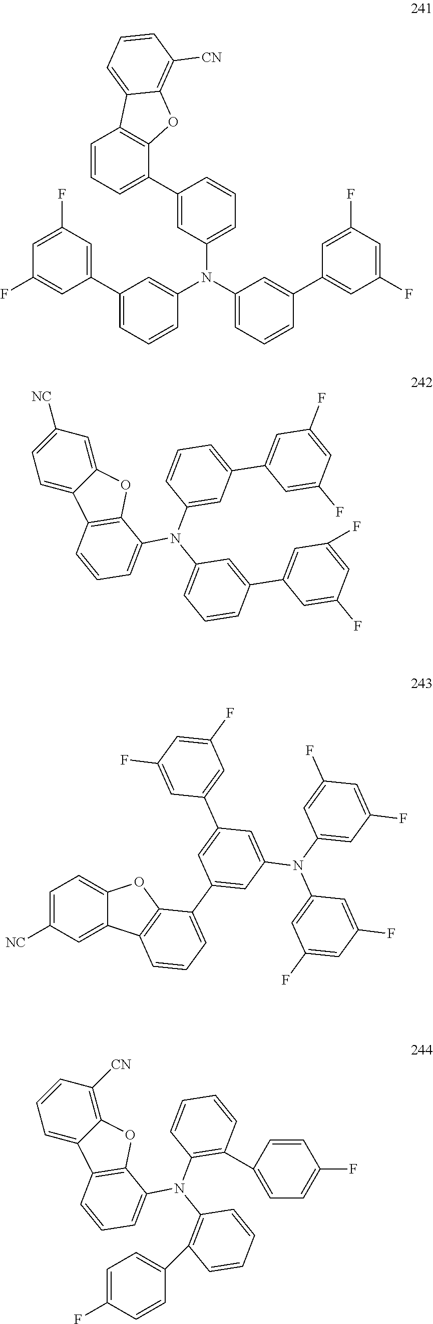 [SMILES: N#Cc1cccc2c1oc1c(-c3cccc(N(c4cccc(-c5cc(F)cc(F)c5)c4)c4cccc(-c5cc(F)cc(F)c5)c4)c3)cccc12 ; N#Cc1ccc2c(c1)oc1c(N(c3cccc(-c4cc(F)cc(F)c4)c3)c3cccc(-c4cc(F)cc(F)c4)c3)cccc12 ; N#Cc1ccc2oc3c(-c4cc(-c5cc(F)cc(F)c5)cc(N(c5cc(F)cc(F)c5)c5cc(F)cc(F)c5)c4)cccc3c2c1 ; N#Cc1cccc2c1oc1c(N(c3ccccc3-c3ccc(F)cc3)c3ccccc3-c3ccc(F)cc3)cccc12]

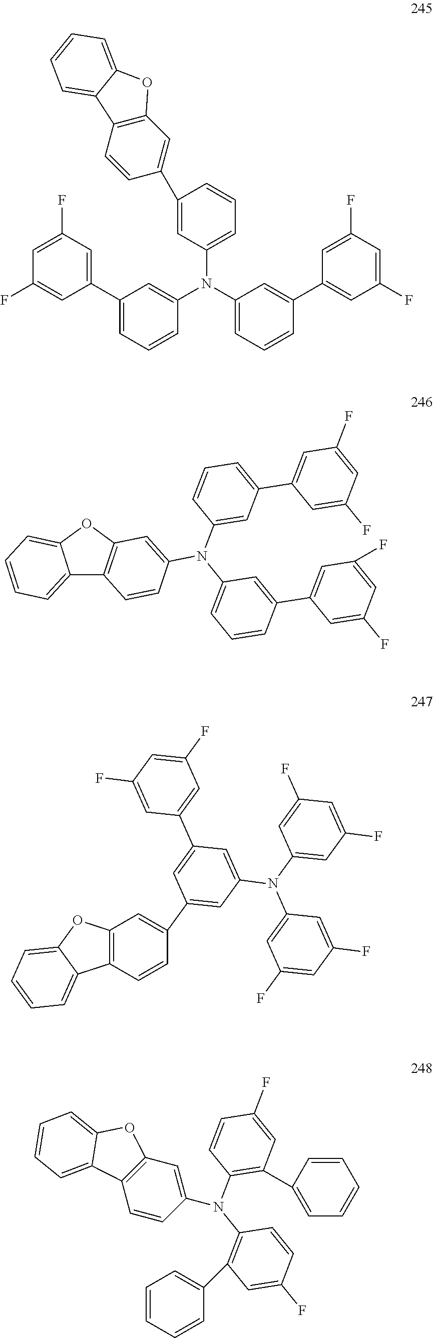 [SMILES: Fc1cc(F)cc(-c2cccc(N(c3cccc(-c4cc(F)cc(F)c4)c3)c3cccc(-c4ccc5c(c4)oc4ccccc45)c3)c2)c1 ; Fc1cc(F)cc(-c2cccc(N(c3cccc(-c4cc(F)cc(F)c4)c3)c3ccc4c(c3)oc3ccccc34)c2)c1 ; Fc1cc(F)cc(-c2cc(-c3ccc4c(c3)oc3ccccc34)cc(N(c3cc(F)cc(F)c3)c3cc(F)cc(F)c3)c2)c1 ; Fc1ccc(N(c2ccc3c(c2)oc2ccccc23)c2ccc(F)cc2-c2ccccc2)c(-c2ccccc2)c1]

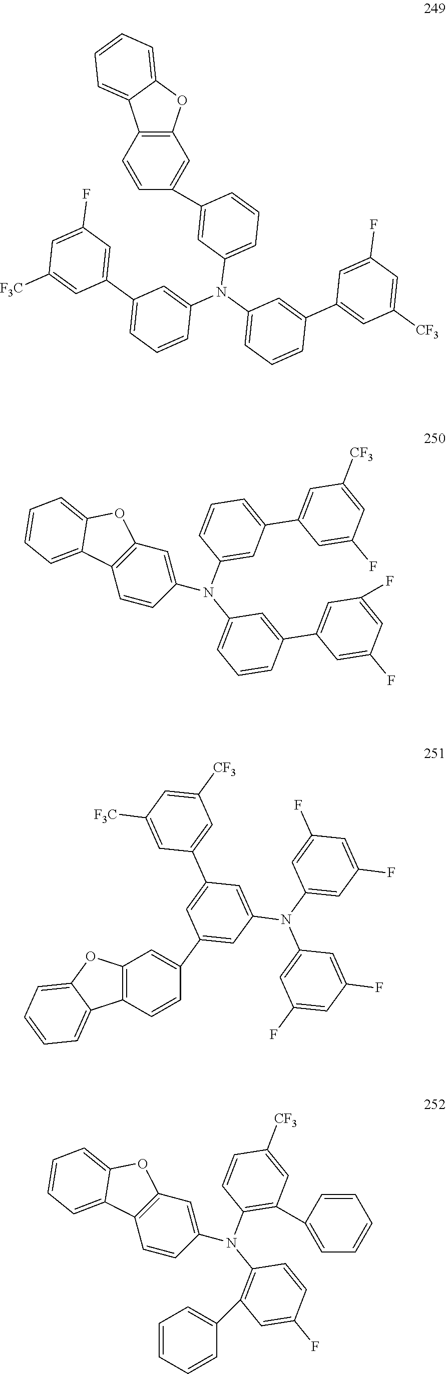 [SMILES: Fc1cc(-c2cccc(N(c3cccc(-c4cc(F)cc(C(F)(F)F)c4)c3)c3cccc(-c4ccc5c(c4)oc4ccccc45)c3)c2)cc(C(F)(F)F)c1 ; Fc1cc(F)cc(-c2cccc(N(c3cccc(-c4cc(F)cc(C(F)(F)F)c4)c3)c3ccc4c(c3)oc3ccccc34)c2)c1 ; Fc1cc(F)cc(N(c2cc(F)cc(F)c2)c2cc(-c3cc(C(F)(F)F)cc(C(F)(F)F)c3)cc(-c3ccc4c(c3)oc3ccccc34)c2)c1 ; Fc1ccc(N(c2ccc3c(c2)oc2ccccc23)c2ccc(C(F)(F)F)cc2-c2ccccc2)c(-c2ccccc2)c1]

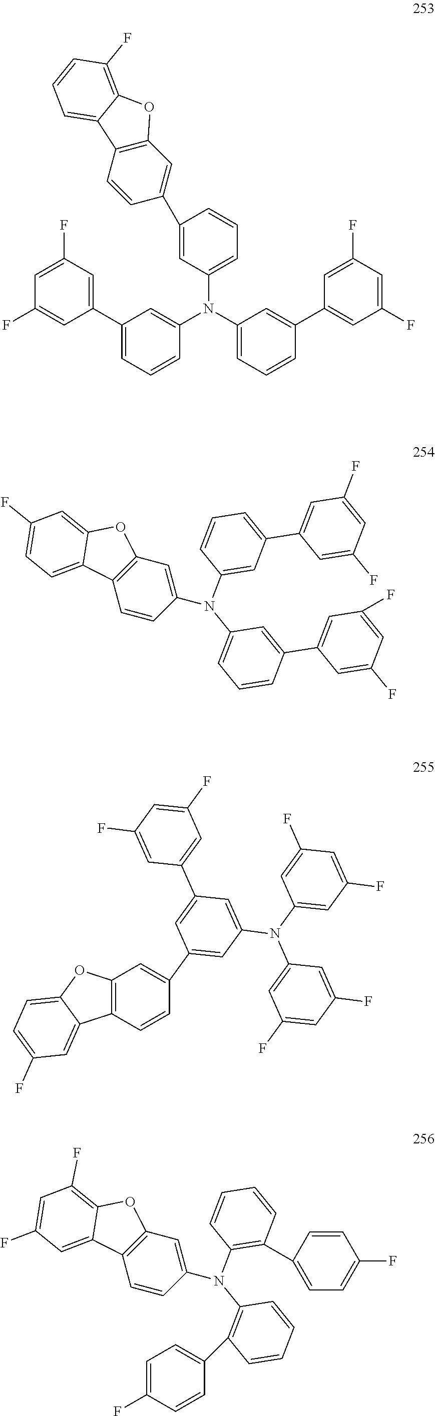 [SMILES: Fc1cc(F)cc(-c2cccc(N(c3cccc(-c4cc(F)cc(F)c4)c3)c3cccc(-c4ccc5c(c4)oc4c(F)cccc45)c3)c2)c1 ; Fc1cc(F)cc(-c2cccc(N(c3cccc(-c4cc(F)cc(F)c4)c3)c3ccc4c(c3)oc3cc(F)ccc34)c2)c1 ; Fc1cc(F)cc(-c2cc(-c3ccc4c(c3)oc3ccc(F)cc34)cc(N(c3cc(F)cc(F)c3)c3cc(F)cc(F)c3)c2)c1 ; Fc1ccc(-c2ccccc2N(c2ccc3c(c2)oc2c(F)cc(F)cc23)c2ccccc2-c2ccc(F)cc2)cc1]

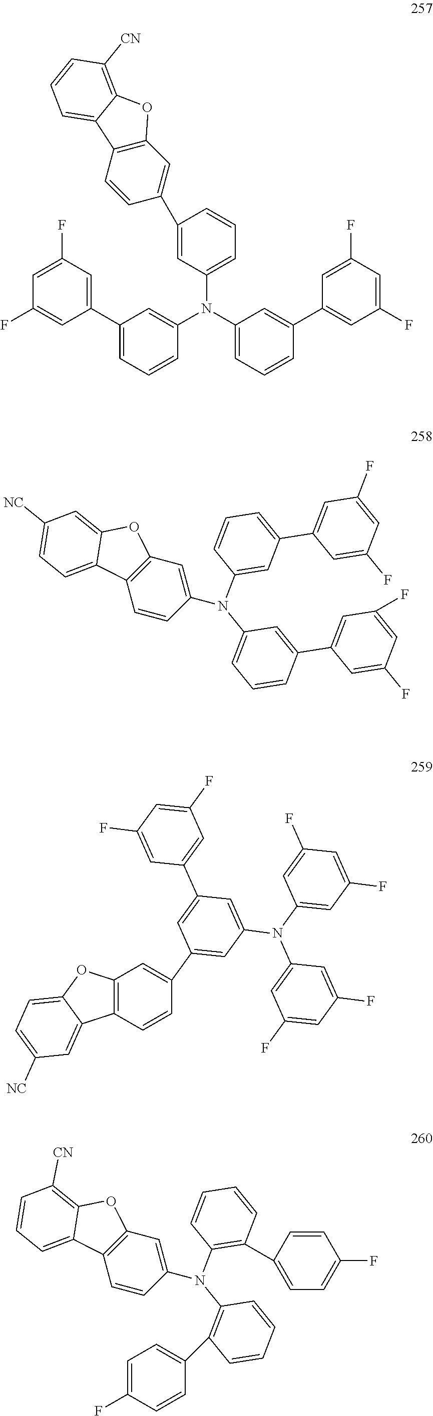 [SMILES: N#Cc1cccc2c1oc1cc(-c3cccc(N(c4cccc(-c5cc(F)cc(F)c5)c4)c4cccc(-c5cc(F)cc(F)c5)c4)c3)ccc12 ; N#Cc1ccc2c(c1)oc1cc(N(c3cccc(-c4cc(F)cc(F)c4)c3)c3cccc(-c4cc(F)cc(F)c4)c3)ccc12 ; N#Cc1ccc2oc3cc(-c4cc(-c5cc(F)cc(F)c5)cc(N(c5cc(F)cc(F)c5)c5cc(F)cc(F)c5)c4)ccc3c2c1 ; N#Cc1cccc2c1oc1cc(N(c3ccccc3-c3ccc(F)cc3)c3ccccc3-c3ccc(F)cc3)ccc12]

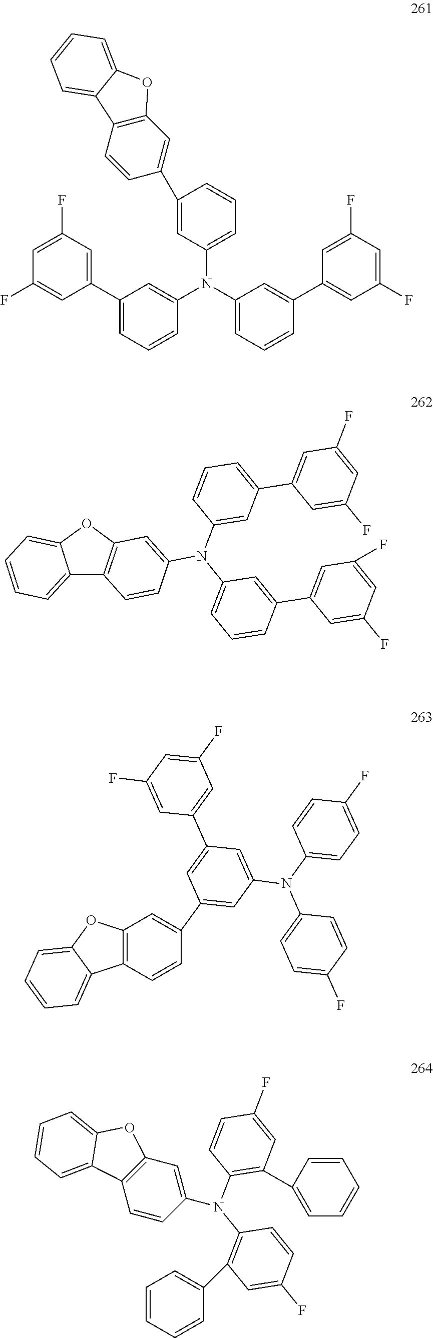 [SMILES: Fc1cc(F)cc(-c2cccc(N(c3cccc(-c4cc(F)cc(F)c4)c3)c3cccc(-c4ccc5c(c4)oc4ccccc45)c3)c2)c1 ; Fc1cc(F)cc(-c2cccc(N(c3cccc(-c4cc(F)cc(F)c4)c3)c3ccc4c(c3)oc3ccccc34)c2)c1 ; Fc1ccc(N(c2ccc(F)cc2)c2cc(-c3cc(F)cc(F)c3)cc(-c3ccc4c(c3)oc3ccccc34)c2)cc1 ; Fc1ccc(N(c2ccc3c(c2)oc2ccccc23)c2ccc(F)cc2-c2ccccc2)c(-c2ccccc2)c1]

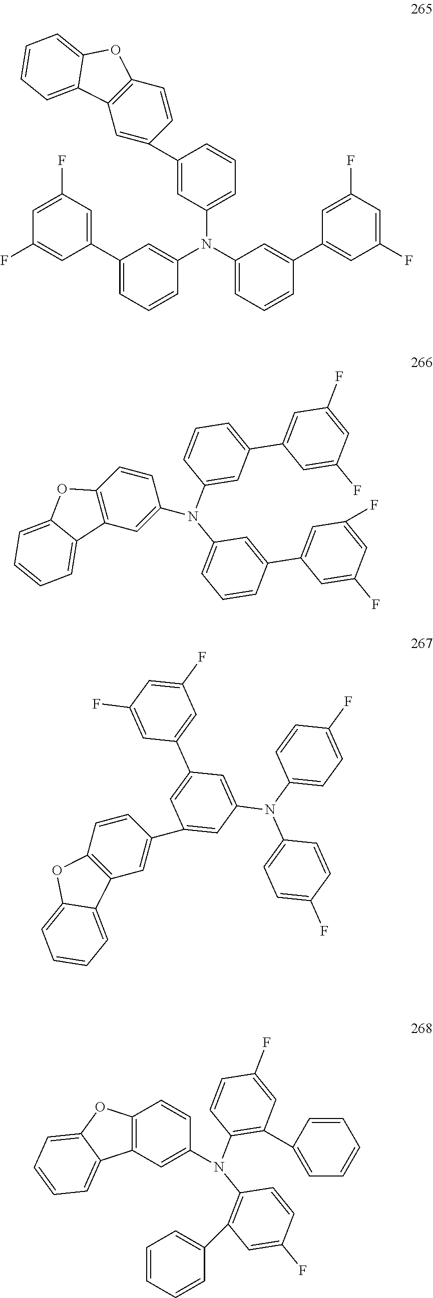 [SMILES: Fc1cc(F)cc(-c2cccc(N(c3cccc(-c4cc(F)cc(F)c4)c3)c3cccc(-c4ccc5oc6ccccc6c5c4)c3)c2)c1 ; Fc1cc(F)cc(-c2cccc(N(c3cccc(-c4cc(F)cc(F)c4)c3)c3ccc4oc5ccccc5c4c3)c2)c1 ; Fc1ccc(N(c2ccc(F)cc2)c2cc(-c3cc(F)cc(F)c3)cc(-c3ccc4oc5ccccc5c4c3)c2)cc1 ; Fc1ccc(N(c2ccc3oc4ccccc4c3c2)c2ccc(F)cc2-c2ccccc2)c(-c2ccccc2)c1]

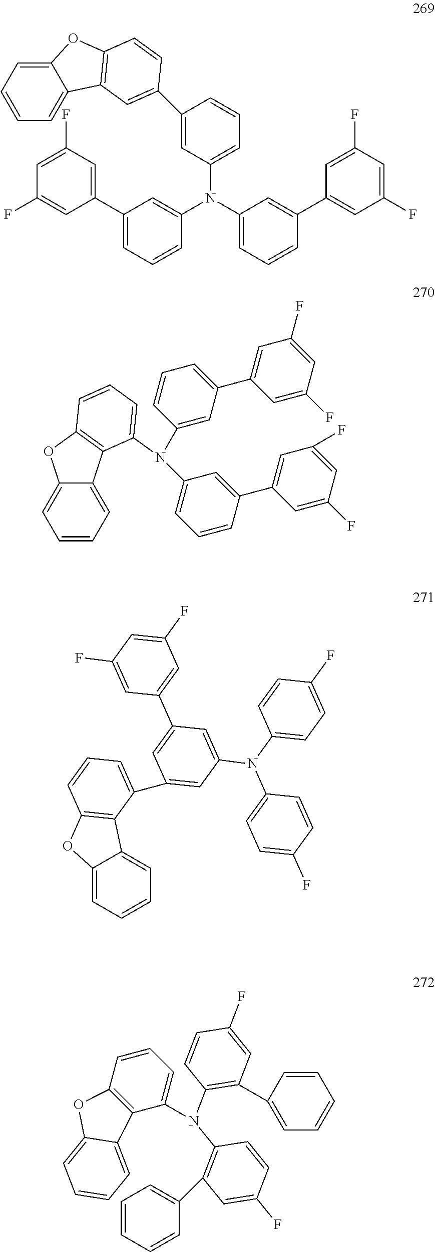 [SMILES: Fc1cc(F)cc(-c2cccc(N(c3cccc(-c4cc(F)cc(F)c4)c3)c3cccc(-c4ccc5oc6ccccc6c5c4)c3)c2)c1 ; Fc1cc(F)cc(-c2cccc(N(c3cccc(-c4cc(F)cc(F)c4)c3)c3cccc4oc5ccccc5c34)c2)c1 ; Fc1ccc(N(c2ccc(F)cc2)c2cc(-c3cc(F)cc(F)c3)cc(-c3cccc4oc5ccccc5c34)c2)cc1 ; Fc1ccc(N(c2ccc(F)cc2-c2ccccc2)c2cccc3oc4ccccc4c23)c(-c2ccccc2)c1]

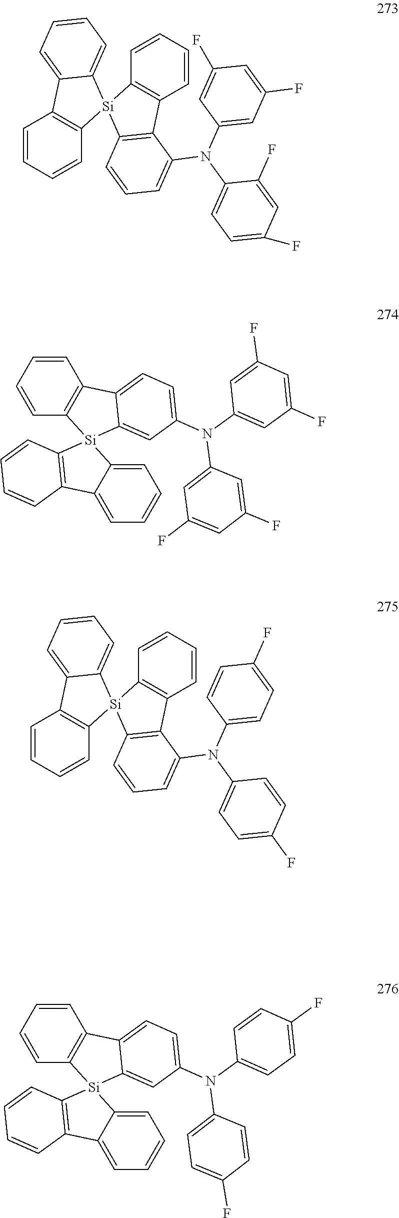 [SMILES: Fc1cc(F)cc(N(c2ccc(F)cc2F)c2cccc3c2-c2ccccc2[Si]32c3ccccc3-c3ccccc32)c1 ; Fc1cc(F)cc(N(c2cc(F)cc(F)c2)c2ccc3c(c2)[Si]2(c4ccccc4-c4ccccc42)c2ccccc2-3)c1 ; Fc1ccc(N(c2ccc(F)cc2)c2cccc3c2-c2ccccc2[Si]32c3ccccc3-c3ccccc32)cc1 ; Fc1ccc(N(c2ccc(F)cc2)c2ccc3c(c2)[Si]2(c4ccccc4-c4ccccc42)c2ccccc2-3)cc1]

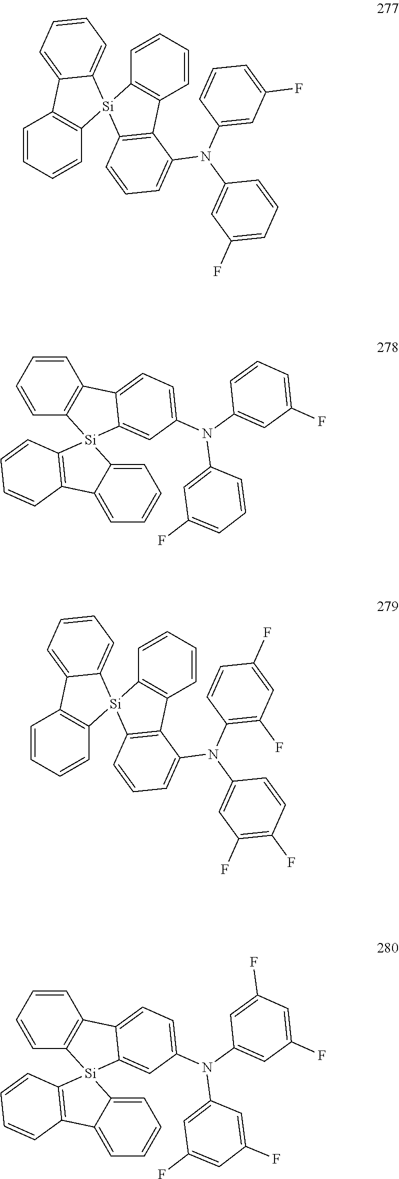 [SMILES: Fc1cccc(N(c2cccc(F)c2)c2cccc3c2-c2ccccc2[Si]32c3ccccc3-c3ccccc32)c1 ; Fc1cccc(N(c2cccc(F)c2)c2ccc3c(c2)[Si]2(c4ccccc4-c4ccccc42)c2ccccc2-3)c1 ; Fc1ccc(N(c2ccc(F)c(F)c2)c2cccc3c2-c2ccccc2[Si]32c3ccccc3-c3ccccc32)c(F)c1 ; Fc1cc(F)cc(N(c2cc(F)cc(F)c2)c2ccc3c(c2)[Si]2(c4ccccc4-c4ccccc42)c2ccccc2-3)c1]

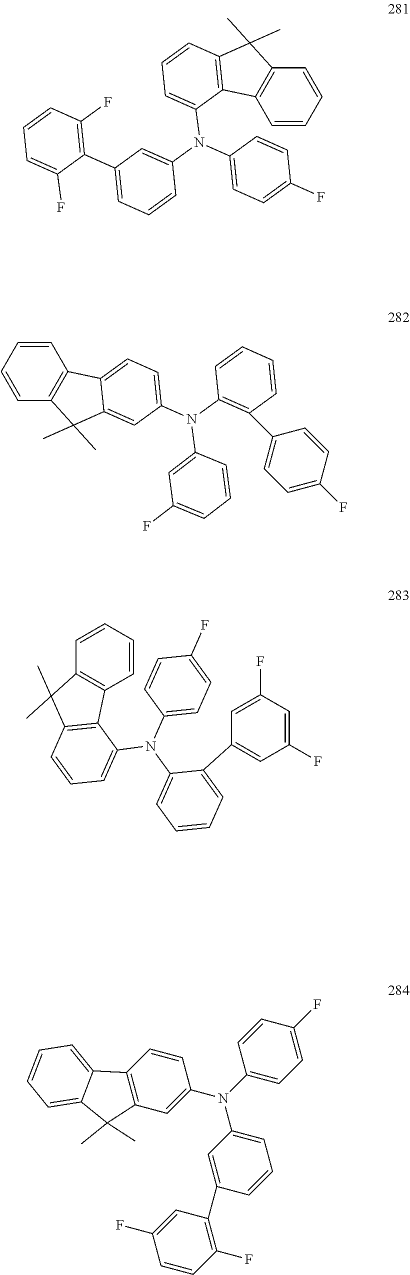 [SMILES: CC1(C)c2ccccc2-c2c(N(c3ccc(F)cc3)c3cccc(-c4c(F)cccc4F)c3)cccc21 ; CC1(C)c2ccccc2-c2ccc(N(c3cccc(F)c3)c3ccccc3-c3ccc(F)cc3)cc21 ; CC1(C)c2ccccc2-c2c(N(c3ccc(F)cc3)c3ccccc3-c3cc(F)cc(F)c3)cccc21 ; CC1(C)c2ccccc2-c2ccc(N(c3ccc(F)cc3)c3cccc(-c4cc(F)ccc4F)c3)cc21]

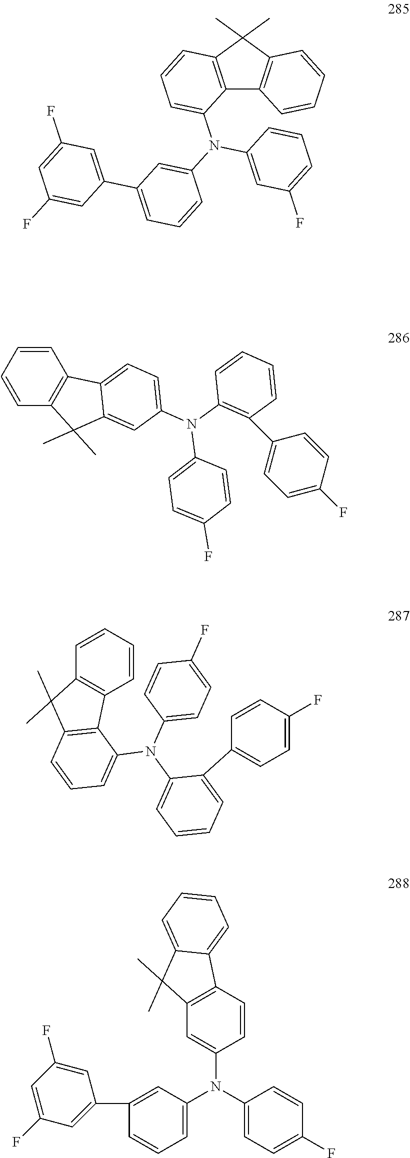 [SMILES: CC1(C)c2ccccc2-c2c(N(c3cccc(F)c3)c3cccc(-c4cc(F)cc(F)c4)c3)cccc21 ; CC1(C)c2ccccc2-c2ccc(N(c3ccc(F)cc3)c3ccccc3-c3ccc(F)cc3)cc21 ; CC1(C)c2ccccc2-c2c(N(c3ccc(F)cc3)c3ccccc3-c3ccc(F)cc3)cccc21 ; CC1(C)c2ccccc2-c2ccc(N(c3ccc(F)cc3)c3cccc(-c4cc(F)cc(F)c4)c3)cc21]

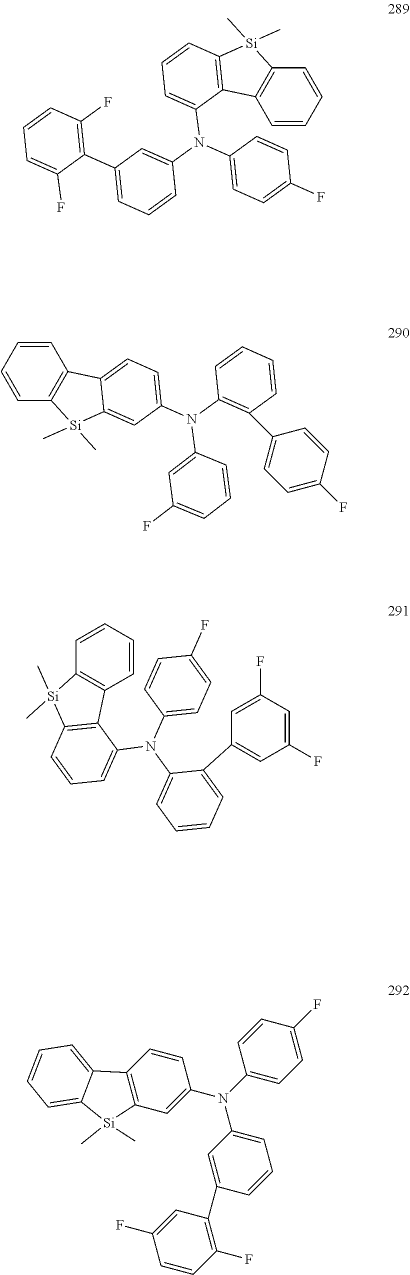 [SMILES: C[Si]1(C)c2ccccc2-c2c(N(c3ccc(F)cc3)c3cccc(-c4c(F)cccc4F)c3)cccc21 ; C[Si]1(C)c2ccccc2-c2ccc(N(c3cccc(F)c3)c3ccccc3-c3ccc(F)cc3)cc21 ; C[Si]1(C)c2ccccc2-c2c(N(c3ccc(F)cc3)c3ccccc3-c3cc(F)cc(F)c3)cccc21 ; C[Si]1(C)c2ccccc2-c2ccc(N(c3ccc(F)cc3)c3cccc(-c4cc(F)ccc4F)c3)cc21]

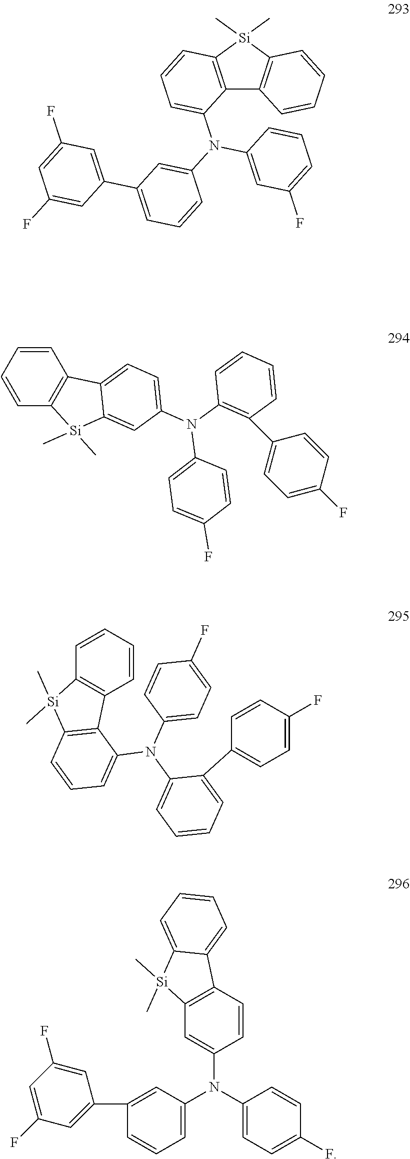 [SMILES: C[Si]1(C)c2ccccc2-c2c(N(c3cccc(F)c3)c3cccc(-c4cc(F)cc(F)c4)c3)cccc21 ; C[Si]1(C)c2ccccc2-c2ccc(N(c3ccc(F)cc3)c3ccccc3-c3ccc(F)cc3)cc21 ; C[Si]1(C)c2ccccc2-c2c(N(c3ccc(F)cc3)c3ccccc3-c3ccc(F)cc3)cccc21 ; C[Si]1(C)c2ccccc2-c2ccc(N(c3ccc(F)cc3)c3cccc(-c4cc(F)cc(F)c4)c3)cc21]

6. An organic electroluminescent material, comprising the organic compound according to claim 1.

7. An organic capping layer material, comprising the organic compound according to claim 1.

8. An organic light-emitting diode (OLED) device, comprising an anode, a cathode, and an organic thin-film layer disposed between the anode and the cathode, wherein a material of the organic thin-film layer comprises the organic compound according to claim 1.

9. An organic light-emitting diode (OLED) device, comprising an anode, a cathode, an organic thin-film layer disposed between the anode and the cathode, and an organic capping layer disposed above the cathode, wherein a material of the organic capping layer comprises the organic compound according to claim 1.

10. The OLED device according to claim 9, wherein the organic capping layer comprises a first capping layer and a second capping layer, the first capping layer comprises the organic compound, and the second capping layer comprises a high-refractive-index material layer having a refractive index greater than or equal to 2.2.

11. The OLED device according to claim 10, wherein the high-refractive-index material layer comprises any one of the following materials:

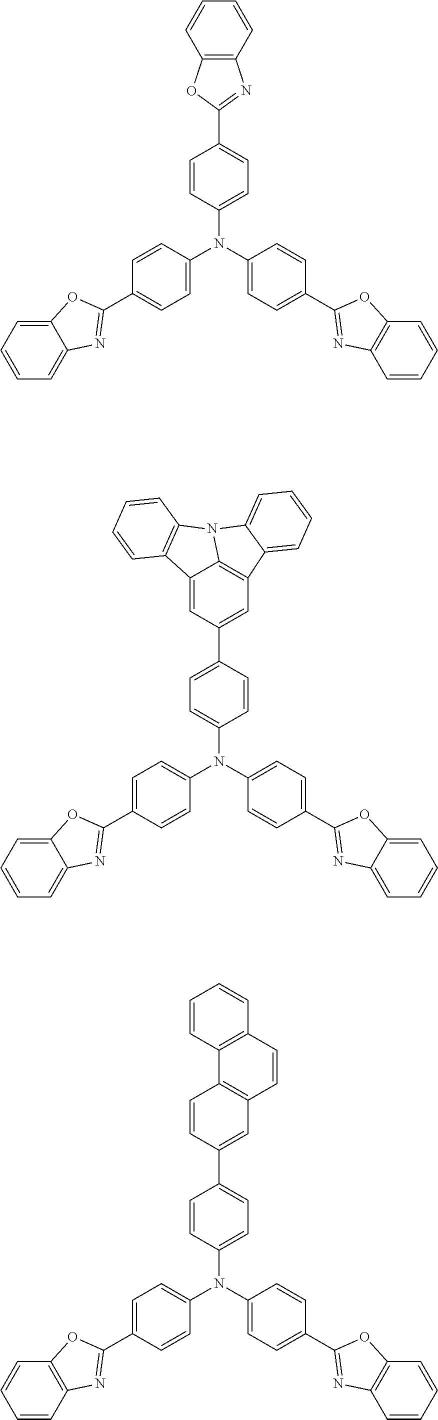 [SMILES: c1ccc2oc(-c3ccc(N(c4ccc(-c5nc6ccccc6o5)cc4)c4ccc(-c5nc6ccccc6o5)cc4)cc3)nc2c1 ; c1ccc2oc(-c3ccc(N(c4ccc(-c5cc6c7ccccc7n7c8ccccc8c(c5)c67)cc4)c4ccc(-c5nc6ccccc6o5)cc4)cc3)nc2c1 ; c1ccc2c(c1)ccc1cc(-c3ccc(N(c4ccc(-c5nc6ccccc6o5)cc4)c4ccc(-c5nc6ccccc6o5)cc4)cc3)ccc12]

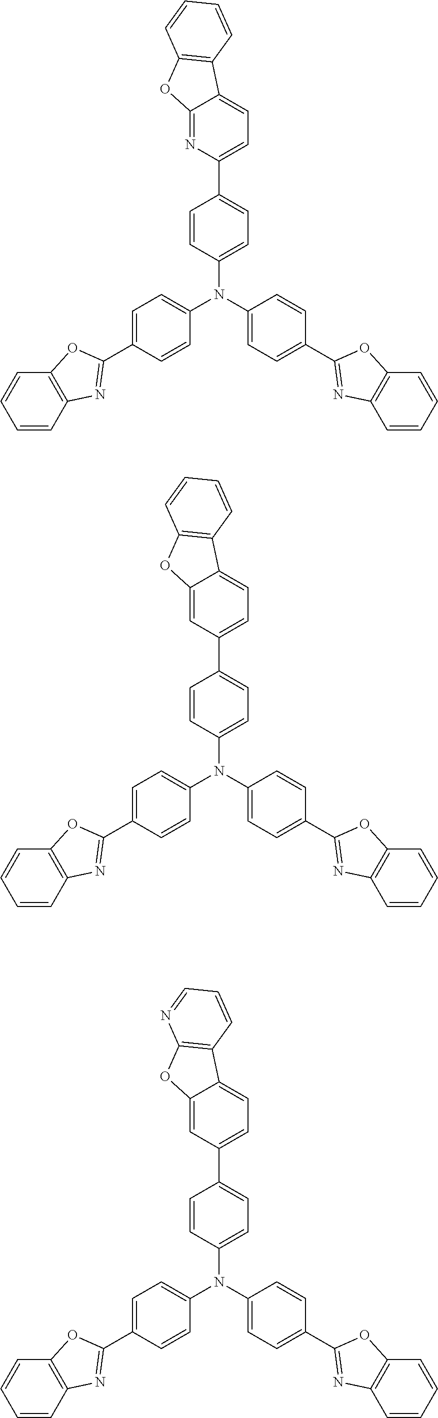 [SMILES: c1ccc2oc(-c3ccc(N(c4ccc(-c5ccc6c(n5)oc5ccccc56)cc4)c4ccc(-c5nc6ccccc6o5)cc4)cc3)nc2c1 ; c1ccc2oc(-c3ccc(N(c4ccc(-c5ccc6c(c5)oc5ccccc56)cc4)c4ccc(-c5nc6ccccc6o5)cc4)cc3)nc2c1 ; c1ccc2oc(-c3ccc(N(c4ccc(-c5ccc6c(c5)oc5ncccc56)cc4)c4ccc(-c5nc6ccccc6o5)cc4)cc3)nc2c1]

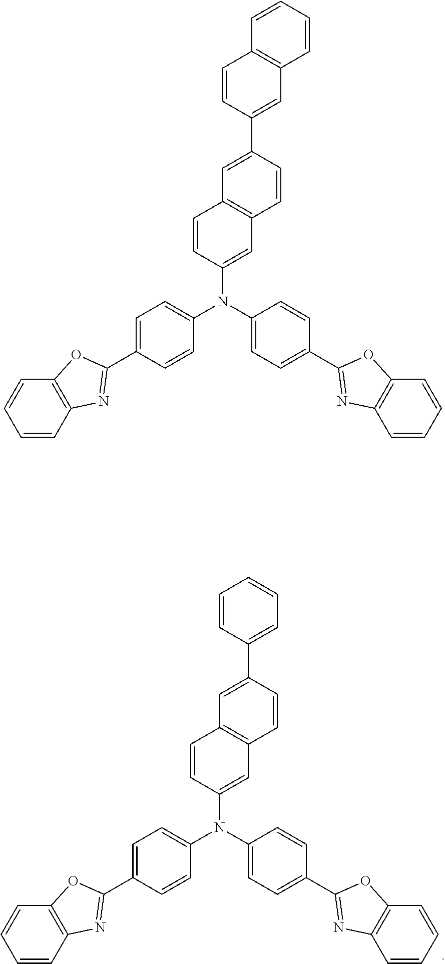 [SMILES: c1ccc2cc(-c3ccc4cc(N(c5ccc(-c6nc7ccccc7o6)cc5)c5ccc(-c6nc7ccccc7o6)cc5)ccc4c3)ccc2c1 ; c1ccc(-c2ccc3cc(N(c4ccc(-c5nc6ccccc6o5)cc4)c4ccc(-c5nc6ccccc6o5)cc4)ccc3c2)cc1]

12. A display panel, comprising the OLED device according to claim 8.

13. An organic light-emitting display device, comprising the display panel according to claim 12.

14. An electronic device, comprising the display panel according to claim 12.
